(19) United States
(12) Patent Application Publication
(10) Pub. No.: US 2024/0084035 A1
(43) Pub. Date: Mar. 14, 2024

(54) COMBINATION THERAPY WITH IMMUNE CELL ENGAGING PROTEINS AND IMMUNOMODULATORS

(71) Applicant: Harpoon Therapeutics, Inc., South San Francisco, CA (US)

(72) Inventors: Mary Ellen MOLLOY, San Francisco, CA (US); Che-Leung LAW, Shoreline, WA (US); Richard J. AUSTIN, San Francisco, CA (US); Bryan D. LEMON, Mountain View, CA (US); Holger WESCHE, San Francisco, CA (US); Wade H. AARON, San Mateo, CA (US)

(21) Appl. No.: 18/457,177

(22) Filed: Aug. 28, 2023

Related U.S. Application Data
(63) Continuation of application No. PCT/US22/19302, filed on Mar. 8, 2022.
(60) Provisional application No. 63/158,721, filed on Mar. 9, 2021.

(51) Int. Cl. C07K 16/30; A61P 35/00; C07K 16/28; C07K 16/40

(52) U.S. Cl. CPC C07K 16/3069; A61P 35/00; C07K 16/2809; C07K 16/2827; C07K 16/2863; C07K 16/2878; C07K 16/3023; C07K 16/40; A61K 2039/507

(57) ABSTRACT

Provided herein are combinations or compositions comprising immunomodulators and immune cell engaging proteins. Also provided herein are methods of use thereof, such as methods of treatment by administering the combinations or compositions comprising immunomodulators and immune cell engaging proteins to a subject. The method may be used to treat a cancer in the subject.

### CROSS-REFERENCE

This application is a continuation of International Application No. PCT/US2022/019302, filed Mar. 8, 2022, which claims the benefit of U.S. Provisional Application No. 63/158,721 filed Mar. 9, 2021, each of which is incorporated by reference herein in its entirety.

### SEQUENCE LISTING

The instant application contains a Sequence Listing which has been submitted electronically in XML file format and is hereby incorporated by reference in its entirety. Said XML copy, created on Aug. 22, 2023, is named 47517-751_301_SL.xml and is 4,731,900 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Cancer is the second leading cause of human death next to coronary disease. Worldwide, millions of people die from cancer every year. In the United States alone, cancer causes the death of well over a half-million people each year, with some 1.4 million new cases diagnosed per year. While deaths from heart disease have been declining significantly, those resulting from cancer generally are on the rise. In the early part of the next century, cancer is predicted to become the leading cause of death.

Moreover, even for those cancer patients that initially survive their primary cancers, common experience has shown that their lives are dramatically altered. Many cancer patients experience strong anxieties driven by the awareness of the potential for recurrence or treatment failure. Many cancer patients experience significant physical debilitations following treatment.

Generally speaking, the fundamental problem in the management of the deadliest cancers is the lack of effective and non-toxic systemic therapies. Cancer is a complex disease characterized by genetic mutations that lead to uncontrolled cell growth. Cancerous cells are present in all organisms and, under normal circumstances, their excessive growth is tightly regulated by various physiological factors.

The selective destruction of an individual cell or a specific cell type is often desirable in a variety of clinical settings. For example, it is a primary goal of cancer therapy to specifically destroy tumor cells, while leaving healthy cells and tissues intact and undamaged. One such method is by inducing an immune response against the tumor, to make immune effector cells such as natural killer (NK) cells or cytotoxic T lymphocytes (CTLs) attack and destroy tumor cells.

### SUMMARY OF THE INVENTION

Provided herein is a combination comprising an immunomodulator and a half-life extended immune cell engaging protein. In some embodiments, the half-life extended immune cell engaging protein comprises an immune cell engaging domain. In some embodiments, the immune cell engaging domain comprises a natural killer (NK) cell engaging domain, a T cell engaging domain, a B cell engaging domain, a dendritic cell engaging domain, a macrophage cell engaging domain, or a combination thereof. In some embodiments, the immune cell engaging domain comprises the T cell engaging domain. In some embodiments, the T cell engaging domain binds a CD3 molecule. In some embodiments, the CD3 molecule is at least one of: a CD3γ molecule, a CD3δ molecule, or a CD3ε molecule.

In some embodiments, the immunomodulator comprises an immunostimulatory antibody agonist of a co-stimulatory receptor. In some embodiments, the immunomodulator comprises an immune checkpoint modulator. In some embodiments, the immune checkpoint modulator is an antagonist of at least one of: programmed cell death 1 (PDCD1, PD1, PD-1), CD274 (CD274, PDL1, PD-L1), PD-L2, cytotoxic T-lymphocyte associated protein 4 (CTLA4, CD152), CD276 (B7H3); V-set domain containing T cell activation inhibitor 1 (VTCN1, B7H4), CD272 (B and T lymphocyte associated (BTLA)), killer cell immunoglobulin like receptor, three Ig domains and long cytoplasmic tail 1 (KIR, CD158E1), lymphocyte activating 3 (LAG3, CD223), hepatitis A virus cellular receptor 2 (HAVCR2, TIMD3, TIM3), V-set immunoregulatory receptor (VSIR, B7H5, VISTA), T cell immunoreceptor with Ig and ITIM domains (TIGIT), programmed cell death 1 ligand 2 (PDCDILG2, PD-L2, CD273), immunoglobulin superfamily member 11 (IGSF11, VSIG3), TNFRSF14 (HVEM, CD270), TNFSF14 (HVEML), PVR related immunoglobulin domain containing (PVRIG, CD112R), galectin 9 (LGALS9), killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1), killer cell lectin like receptor C1 (KLRC1, NKG2A, CD159A), killer cell lectin like receptor D1 (KLRD1, CD94), killer cell lectin like receptor G1 (KLRG1, CLEC15A, MAFA, 2F1), sialic acid binding Ig like lectin 7 (SIGLEC7), SIGLEC, sialic acid binding Ig like lectin 9 (SIGLEC9), CEACAM (e.g., CEACAM-1, CEACAM-3 and/or CEACAM-5), VISTA, LAIR1, CD160, 2B4, CD80, CD86, B7-H1, B7-H3 (CD276), B7-H4 (VTCN1), HVEM (TNFRSF14 or CD270), KIR, A2AR, A2BR, MHC class I, MHC class II, GAL9, adenosine, TGFR (e.g., TGFR beta), CD94/NKG2A, IDO, TDO, CD39, CD73, GARP, CD47, PVRIG, CSF1R, and NOX, or any combination thereof.

In some embodiments, the immune checkpoint modulator is an antagonist of PD-1 and is selected from the group consisting of. Pembrolizumab (humanized antibody), Pidilizumab (CT-011, monoclonal antibody, binds DLL1 and PD-1), Spartalizumab (PDR001, monoclonal antibody), Nivolumab (BMS-936558, MDX-1106, human IgG4 monoclonal antibody), MEDI0680 (AMP-514, monoclonal antibody), Cemiplimab (REGN2810, monoclonal antibody), Dostarlimab (TSR-042, monoclonal antibody), Sasanlimab (PF-06801591, monoclonal antibody), Tislelizumab (BGB-A317, monoclonal antibody), BGB-108 (antibody), Tislelizumab (BGB-A317, antibody), Camrelizumab (INCSHR1210, SHR-1210), AMP-224, Zimberelimab (AB122, GLS-010, WBP-3055, monoclonal antibody), AK-103 (HX-008, monoclonal antibody), AK-105 (anti-PD-1 antibody), CS1003 (monoclonal antibody), HLX10 (monoclonal antibody), Retifanlimab (MGA-012, anti-PD-1 monoclonal antibody), BI-754091 (antibody), Balstilimab (AGEN2034, PD-1 antibody), toripalimab (JS-001, antibody), cetrelimab (JNJ-63723283, anti-PD-1 antibody), genolimzumab (CBT-501, anti-PD-1 antibody), LZM009 (anti-PD-1 monoclonal antibody), Prolgolimab (BCD-100, anti-PD-1 monoclonal antibody), Sym021 (antibody), ABBV-181 (antibody), BAT-1306 (antibody), JTX-4014, sintilimab (IBI-308), Tebotelimab (MGD013, PD-1/LAG-3 bispecific), MGD-019 (PD-1/CTLA4 bispecific antibody), KN-046 (PD-1/CTLA4 bispecific antibody), MEDI-5752 (CTLA4/PD-1 bispecific antibody), RO7121661 (PD-1/TIM-3 bispecific antibody), XmAb207I7 (PD-1/CTLA4 bispecific antibody), and AK-104 (CTLA4/PD-1 bispecific antibody).

In some embodiments, the immune checkpoint modulator is Pembrolizumab. In some embodiments, the immune checkpoint modulator is an antibody that binds to PD-L1 and is selected from the group consisting of Atezolizumab (MPDL3280A, monoclonal antibody), Avelumab (MSB0010718C, monoclonal antibody), Durvalumab (MEDI-4736, human immunoglobulin G1 kappa (IgG1κ) monoclonal antibody), Envafolimab (KN035, single-domain PD-L1 antibody), AUNP12, CA-170 (small molecule targeting PD-L1 and VISTA), BMS-986189 (macrocyclic peptide), BMS-936559 (Anti-PD-L1 antibody), Cosibelimab (CK-301, monoclonal antibody), LY3300054 (antibody), CX-072 (antibody), CBT-502 (antibody), MSB-2311 (antibody), BGB-A333 (antibody), SHR-1316 (antibody), CS1001 (WBP3155, antibody), HLX-20 (antibody), KL-A167 (HBM 9167, antibody), STI-A1014 (antibody), STI-A1015 (IMC-001, antibody), BCD-135 (monoclonal antibody), FAZ-053 (antibody), CBT-502 (TQB2450, antibody), MDX1105-01 (antibody), FS-118 (LAG-3/PD-L1, bispecific antibody), M7824 (anti-PD-L1/TGF-β receptor II fusion protein), CDX-527 (CD27/PD-L1 bispecific antibody), LY3415244 (TIM3/PD-L1 bispecific antibody), INBRX-105 (4-1BB/PD-L1 bispecific antibody).

In some embodiments, the immune checkpoint modulator is Atezolizumab. In some embodiments, the immune checkpoint modulator is an anti-CD39 antibody. In some embodiments, the anti-CD39 antibody is IPH5201. In some embodiments, the immune checkpoint modulator is an anti-CD73 antibody. In some embodiments, the anti-CD73 antibody is IPH5301. In some embodiments, the immunomodulator is an inhibitor of at least one of: A2AR, CD39, or CD73. In some embodiments, the inhibitor is a small molecule inhibitor. In some embodiments, the immune checkpoint modulator comprises an immune checkpoint activator. In some embodiments, the immune checkpoint activator is an agonist of CD27, CD70, CD40, CD40LG, TNF receptor superfamily member 4 (TNFRSF4, OX40); TNF superfamily member 4 (TNFSF4, OX40L), GITR (TNF receptor superfamily member 18, TNFRSF18, CD357), TNFSF18 (GITRL), CD137 (TNFRSF9, tumor necrosis factor receptor superfamily member 9, 4-1BB, ILA, induced by lymphocyte activation), CD137L (TNFSF9), CD28, CD278 (inducible T cell co-stimulator, ICOS), inducible T cell co-stimulator ligand (ICOSLG, B7H2), CD80 (B7-1), nectin cell adhesion molecule 2 (NECTIN2, CD112), CD226 (DNAM-1), Poliovirus receptor (PVR) cell adhesion molecule (PVR, CD155), CD16, killer cell lectin like receptor K1 (KLRKI1, NKG2D, CD314), or SLAM family member 7 (SLAMF7). In some embodiments, the agonist is an antibody or an antigen-binding fragment thereof.

In some embodiments, the half-life extended immune cell engaging protein comprises a first domain (A), a second domain (B), and a third domain (D), wherein (i) the first domain (A) is the T cell engaging domain and specifically binds to human CD3, (ii) the second domain (B) specifically binds to human serum albumin (HSA), and (iii) the third domain (C) specifically binds to a target antigen; wherein the domains are linked in one of the following orders: H₂N-(C)-(B)-(A)-COOH, H₂N-(A)-(B)-(C)-COOH, H₂N-(B)-(A)-(C)-COOH, H₂N-(C)-(A)-(B)-COOH, H₂N-(A)-(C)-(B)-COOH, H₂N-(B)-(C)-(A)-COOH, or by linkers L1 and L2 in one of the following orders: H₂N-(C)-L1-(B)-L2(A)-COOH, H₂N-(A)-L1-(B)-L2-(C)-COOH, H₂N-(B)-L1-(A)-L2-(C)-COOH, H₂N-(C)-L1-(A)-L2-(B)-COOH, H₂N-(A)-L1-(C)-L2(B)-COOH, H₂N-(B)-L1-(C)-L2-(A)-COOH.

In some embodiments, target antigen in a tumor antigen. In some embodiments, the third domain specifically binds to a target antigen selected from the group consisting of: CD19 (B-lymphocyte antigen CD19, B-Lymphocyte Surface Antigen B4, T-Cell Surface Antigen Leu-12, CVID3), PSMA (prostate specific membrane antigen), MSLN (mesothelin), BCMA (B-cell maturation antigen), DLL3 (Delta-like ligand 3), EGFR (epidermal growth factor receptor), FLT3 (FMS-like tyrosine kinase 3), CD20 (B-lymphocyte antigen CD20, MS4A1, B1, Bp35, CVID5, LEU-16, MS4A2, S7, membrane spanning 4-domains A1), CD22 (SIGLEC-2, SIGLEC2), CD25 (IL2RA, interleukin-2 receptor alpha chain), CD27 (S152, S152. LPFS2, T14, TNFRSF7, Tp55), CD30 (TNFRSF8), CD33 (Siglec-3, sialic acid binding Ig-like lectin 3, SIGLEC3, SIGLEC-3, gp67, p67), CD37 (GP52-40, TSPAN26), CD38 (cyclic ADP ribose hydrolase, ADPRC1, ADPRC 1), CD40 (Bp50, CDW40, TNFRSF5, p50), CD44 (HCAM, homing cell adhesion molecule), Pgp-1 (phagocytic glycoprotein-1), Hermes antigen, lymphocyte homing receptor, ECM-III, and HUTCH-1), CD48 (BLAST-1, B-lymphocyte activation marker, SLAMF2, signaling lymphocytic activation molecule 2), CD52 (CAMPATH-1 antigen), CD70, CD73 (NT5E, ecto-5′-nucleotidase), CD39 (ENTPD1, Ectonucleoside triphosphate diphosphohydrolase-1), CD74 (HLA class II histocompatibility antigen gamma chain, HLA-DR antigens-associated invariant chain), CD79b (immunoglobulin-associated beta), CD80 (B7-1), CD86 (B7-2), CD123 (IL3RA, interleukin-3 receptor), CD133 (PROM1), CD137 (TNFRSF9, tumor necrosis factor receptor superfamily member 9, 4-1BB, ILA, induced by lymphocyte activation), CD138 (SDC1), alpha fetoprotein (AFP), c-Met; c-Kit; CD371 (CLEC12A, C-type lectin domain family 12 member A, CLL1)); CD370 (CLEC9A, C-type lectin domain containing 9A); cadherin 3 (CDH3, p-cadherin, PCAD); carbonic anhydrase 6 (CA6); carbonic anhydrase 9 (CA9, CAIX); carcinoembryonic antigen related cell adhesion molecule 3 (CEACAM3); carcinoembryonic antigen related cell adhesion molecule 5 (CEACAM5); CD66c (CEACAM6, carcinoembryonic antigen related cell adhesion molecule 6); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EPCAM); erb-b2 receptor tyrosine kinase 2 (ERBB2, HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRa); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); CD352 (SLAMF6, SLAM family member 6); CD319 (SLAMF7, SLAM family member 7, 19A, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6, LIV1); STEAP family member 1 (STEAP1); STEAP family member 2 (STEAP2); CD134 (TNFRSF4, TNF receptor superfamily member 4, OX40); CD137L (TNFSF9, TNF superfamily member 9, 4-1BB-L); CD261 (TNFRSF10A, TNF receptor superfamily member 10a, DR4, TRAILR1); CD262 (TNFRSF10B, TNF receptor superfamily member 10b, DR5, TRAILR2); CD267 (TNFRSF13B, TNF receptor superfamily member 13B, TACI, IGAD2); CD269 (TNFRSF17, TNF receptor superfamily member 17, BCMA,); CD357 (TNFRSF18, TNF receptor superfamily member 18 GITR); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GM1; Sialyl Lewis adhesion molecule (sLe); ROR1; CD30; and Lewis Y antigen.

In some embodiments, the third domain is a single domain antibody that specifically binds to PSMA. In some embodiments, the third domain comprises a CDR1 comprising an amino acid sequence selected from the group consisting of SEQ ID NOS: 462-465, a CDR2 comprising an amino acid sequence selected from the group consisting of SEQ ID NOS: 466-472, and a CDR3 comprising an amino acid sequence selected from the group consisting of SEQ ID NOS: 474-475. In some embodiments, the CDR1 comprises the amino acid sequence of SEQ ID NO: 462, the CDR2 comprises the amino acid of SEQ ID NO: 473, and the CDR3 comprises the amino acid sequence of SEQ ID NO: 474. In some embodiments, the third domain comprises an amino acid sequence selected from the group consisting of SEQ ID NOS: 476-489. In some embodiments, the third domain comprises the amino acid sequence of SEQ ID NO: 489.

In some embodiments, the third domain is a single domain antibody that specifically binds to MSLN. In some embodiments, the third domain comprises a CDR1 comprising an amino acid sequence selected from the group consisting of SEQ ID NOS: 490-528, a CDR2 comprising an amino acid selected from the group consisting of SEQ ID NOS: 529-567, and a CDR3 comprising an amino acid selected from the group consisting of SEQ ID NOS: 568-606. In some embodiments, the CDR1 comprises the amino acid sequence of SEQ ID NO: 523, the CDR2 comprises the amino acid of SEQ ID NO: 562, and the CDR3 comprises the amino acid sequence of SEQ ID NO: 601. In some embodiments, the third domain comprises an amino acid sequence selected from the group consisting of SEQ ID NOS: 607-650. In some embodiments, the third domain comprises the amino acid sequence of SEQ ID NO: 647. In some embodiments, the third domain is a single domain antibody that specifically binds to BCMA. In some embodiments, the third domain CDR1 comprises comprising an amino acid selected from the group consisting of SEQ ID NOS: 1-115, a CDR2 comprising an amino acid selected from the group consisting of SEQ ID NOS: 116-230, and a CDR3 comprising an amino acid selected from the group consisting of SEQ ID NOS: 231-345. In some embodiments, the CDR1 comprises the amino acid sequence of SEQ ID NO: 73, the CDR2 comprises the amino acid of SEQ ID NO: 188, and the CDR3 comprises the amino acid sequence of SEQ ID NO: 303. In some embodiments, the third domain comprises an amino acid sequence selected from the group consisting of SEQ ID NOS: 346-461. In some embodiments, the third domain comprises the amino acid sequence of SEQ ID NO: 383.

In some embodiments, the third domain is a single domain antibody that specifically binds to DLL3. In some embodiments, the third domain comprises a CDR1 comprising an amino acid selected from the group consisting of SEQ ID NOS: 1751-2193, a CDR2 comprising an amino acid selected from the group consisting of SEQ ID NOS: 2194-2636, and a CDR3 comprising an amino acid selected from the group consisting of SEQ ID NOS: 2637-3080. In some embodiments, the CDR1 comprises the amino acid sequence of SEQ ID NO: 2182, the CDR2 comprises the amino acid of SEQ ID NO: 2625, and the CDR3 comprises the amino acid sequence of SEQ ID NO: 3069. In some embodiments, the third domain comprises an amino acid sequence selected from the group consisting of SEQ ID NOS: 1308-1750. In some embodiments, the third domain comprises the amino acid sequence of SEQ ID NO: 1739. In some embodiments, the third domain is a single domain antibody that specifically binds to EGFR. In some embodiments, the third domain comprises a CDR1 comprising an amino acid selected from the group consisting of SEQ ID NOS: 651-699, a CDR2 comprising an amino acid selected from the group consisting of SEQ ID NOS: 700-748, a CDR3 comprising an amino acid selected from the group consisting of SEQ ID NOS: 479-797. In some embodiments, the third domain comprises an amino acid sequence selected from the group consisting of SEQ ID NOS: 798-846.

In some embodiments, the third domain is a single domain antibody that specifically binds to FLT3. In some embodiments, the third domain comprises a CDR1 comprising an amino acid selected from the group consisting of SEQ ID NOS: 1080-1155 and 3497-3498, a CDR2 comprising an amino acid selected from the group consisting of SEQ ID NOS: 1156-1231, and 3499-3500, a CDR3 comprising an amino acid selected from the group consisting of SEQ ID NOS: 1232-1307, and 3501-3502. In some embodiments, the CDR1 comprises an amino acid sequence selected from the group consisting of SEQ ID NOS: 1150, 1152, 3497, and 3498; the CDR2 comprises an amino acid sequence selected from the group consisting of SEQ ID NOS: 1226, 1228, 3499, and 3500; the CDR3 comprises an amino acid sequence selected from the group consisting of SEQ ID NOS: 1302, 1304, 3501, and 3502. In some embodiments, the third domain comprises an amino acid sequence selected from the group consisting of SEQ ID NOS: 1004-1079 and 3495-3496. In some embodiments, the third domain comprises an amino acid sequence selected from the group consisting of: SEQ ID NOS: 1074, 1076, 3495, and 3496. In some embodiments, the third domain is a single domain antibody that specifically binds to EpCAM. In some embodiments, the third domain comprises a CDR1 comprising an amino acid selected from the group consisting of SEQ ID NOS: 847-884, a CDR2 comprising an amino acid selected from the group consisting of SEQ ID NOS: 885-922, a CDR3 comprising an amino acid selected from the group consisting of SEQ ID NOS: 923-960. In some embodiments, the CDR1 comprises the amino acid sequence of SEQ ID NO: 874 or 863, the CDR2 comprises the amino acid of SEQ ID NO: 885 or 901, and the CDR3 comprises the amino acid sequence of SEQ ID NO: 923 or 939. In some embodiments, the third domain comprises a sequence selected from the group consisting of SEQ ID NOS: 961-1003. In some embodiments, the third domain comprises the amino acid sequence of SEQ ID NO: 999 or 1003. In some embodiments, the first domain comprises a single-chain variable fragment (scFv) specific to human CD3. In some embodiments, the scFv specific to human CD3 comprises a variable heavy chain region (VH), a variable light chain region (VL), and a linker, wherein VH comprises complementarity determining regions HC CDR1, HC CDR2, and HC CDR3, and wherein VL comprises complementarity determining regions LC CDR1, LC CDR2, and LC CDR3. In some embodiments, the HC CDR1 comprises an amino acid sequence selected from the group consisting of SEQ ID NOS: 3081, and 3087-3098, the HC CDR2 comprises an amino acid sequence selected from the group consisting of SEQ ID NOS: 3082, and 3099-3109, and the HC CDR3 comprises an amino acid sequence selected from the group consisting of SEQ ID NOS: 3083, and 3110-3119. In some embodiments, the HC CDR1 comprises the amino acid sequence of SEQ ID NO: 3097, the HC CDR2 comprises the amino acid sequence of SEQ ID NO: 3108, the HC CDR3 comprises the amino acid sequence of SEQ ID NO: 3110. In some embodiments, the LC CDR1 comprises an amino acid sequence selected from the group consisting of SEQ ID NOS: 3084, and 3120-3132, the LC CDR2 comprises an amino acid sequence selected from the group consisting of SEQ ID NOS: 3085, and 3099-3109, and the LC CDR3 comprises an amino acid sequence selected from the group consisting of SEQ ID NOS: 3086, and 3146-3152. In some embodiments, the LC CDR1 comprises the amino acid sequence of SEQ ID NO: 3120, the LC CDR2 comprises the amino acid sequence of SEQ ID NO: 3145, the LC CDR3 comprises the amino acid sequence of SEQ ID NO: 3146.

In some embodiments, the first domain comprises an amino acid sequence selected from the group consisting of SEQ ID NOS: 3153-3169. In some embodiments, the first domain comprises the amino acid sequence of SEQ ID NO: 3153. In some embodiments, the linker comprises an amino acid sequence GGGGSGGGGSGGGGS (SEQ ID NO: 3199).

In some embodiments, the second domain comprises a single domain antibody (sdAb) which specifically binds to HSA. In some embodiments, the sdAb which specifically binds to HSA comprises complementarity determining regions CDR1, CDR2, and CDR3, wherein the CDR1 comprises an amino acid sequence selected from the group consisting of SEQ ID NOS: 3170 and 3173-3175, the CDR2 comprises an amino acid sequence selected from the group consisting of SEQ ID NOS: 3171 and 3176-3181, and the CDR3 comprises an amino acid sequence selected from the group consisting of SEQ ID NOS: 3172 and 8182-3183. In some embodiments, the CDR1 comprises an amino acid sequence of SEQ ID NO: 3174, the CDR2 comprises an amino acid of SEQ ID NO: 3178, and the CDR3 comprises an amino acid sequence of SEQ ID NO: 3183. In some embodiments, the second domain comprises an amino acid sequence selected from the group consisting of SEQ ID NOS: 3184-3193. In some embodiments, the second domain comprises the amino acid sequence of SEQ ID NO: 3190. In some embodiments, the linkers L1 and L2 are each, independently, (GS)ₙ (SEQ ID NO: 3525), (GGS)ₙ(SEQ ID NO: 3526), (GGGS)ₙ(SEQ ID NO: 3527), (GGSG)ₙ (SEQ ID NO: 3528), (GGSGG)ₙ (SEQ ID NO: 3194), or (GGGGS)ₙ (SEQ ID NO: 3195), wherein n is 1, 2, 3, 4, 5, 6, 7, 8, 9, or 10. In some embodiments, linkers L1 and L2 are each, independently, (GGGGS)₄ (SEQ ID NO: 3198) or (GGGGS)₃ (SEQ ID NO: 3199).

In some embodiments, the immunomodulator and the half-life extended immune cell engaging protein are in a single pharmaceutical composition. In some embodiments, the immunomodulator and the half-life extended immune cell engaging protein are in separate pharmaceutical compositions.

Provided herein is a composition comprising an immunomodulator and a half-life extended immune cell engaging protein.

Provided herein is a method for the treatment or amelioration of a disease in a subject in need thereof, comprising administering to the subject a combination or a composition provided herein. In some embodiments, the disease is a cancer.

Provided herein is a method for increasing survival in a subject suffering from a cancer, the method comprising administering to the subject a combination or a composition provided herein. Provided herein is a method of reducing tumor size, the method comprising administering to a subject from a cancer a combination or a composition provided herein.

In some embodiments, the cancer is selected from the group consisting of: a mesothelioma, a prostate cancer, a breast cancer, a brain cancer, a bladder cancer, a pancreatic carcinoma, a renal cancer, a solid tumor, a liver cancer, a leiomyosarcoma, an endometrium cancer, a breast cancer, a female reproductive system cancer, an ovarian carcinoma, a soft tissue sarcoma, a gastric cancer, a digestive/gastrointestinal cancer, a colorectal cancer, a glioblastoma multiforme, a head and neck cancer, a squamous cell carcinoma, a colon cancer, a gastric cancer, a rhabdomyosarcoma, an adrenal cancer, a lung cancer, an esophageal cancer, a colon cancer, a lung cancer, a non-small cell lung carcinoma (NSCLC), a neuroblastoma, a melanoma, glioblastoma multiforme, an ovarian cancer, an endocrine cancer, a respiratory/thoracic cancer, an anal cancer, a gastro-esophageal cancer, a thyroid cancer, a cervical cancer, an endometrial cancer, a hematological cancer, a leukemia, a lymphocytic leukemia, a multiple myeloma, a lymphoma, a Hodgkin's lymphoma, a non-Hodgkin's lymphoma, a lymphocytic leukemia, an anaplastic large-cell lymphoma (ALCL), or a myeloid leukemia. In some embodiments, the cancer is the prostate cancer. In some embodiments, the cancer is the ovarian carcinoma. In some embodiments, the cancer is the pancreatic carcinoma. In some embodiments, the cancer is the mesothelioma. In some embodiments, the cancer is the lung cancer. In some embodiments, the administering the combination results in an increased therapeutic benefit compared to administering the immunomodulator alone without the half-life extended immune cell engaging protein. In some embodiments, the administering the combination results in an increased therapeutic benefit compared to administering the half-life extended immune cell engaging protein alone without the immunomodulator. In some embodiments, the half-life extended immune cell engaging protein and the immunomodulator are administered concurrently. In some embodiments, the half-life extended immune cell engaging protein and the immunomodulator are administered sequentially.

Provided herein is a method of increasing the sensitivity of a subject to a therapy comprising administering an immune checkpoint inhibitor, the method comprising administering to the subject a half-life extended immune cell engaging protein comprising: (i) a first domain (A) which specifically binds to human CD3, (ii) a second domain (B) which specifically binds to human serum albumin (HSA), and (iii) a third domain (C) which specifically binds to a target antigen. In some embodiments, the administering the half-life extended immune cell engaging protein increases the concentration of an immune checkpoint protein targeted by the immune checkpoint inhibitor, in the subject.

In some embodiments, the immune checkpoint protein is PD-1. In some embodiments, the immune checkpoint inhibitor comprises an antibody selected from the group consisting of: and is selected from the group consisting of: Pembrolizumab (humanized antibody), Pidilizumab (CT-011, monoclonal antibody, binds DLL1 and PD-1), Spartalizumab (PDR001, monoclonal antibody), Nivolumab (BMS-936558, MDX-1106, human IgG4 monoclonal antibody), MEDI0680 (AMP-514, monoclonal antibody), Cemiplimab (REGN2810, monoclonal antibody), Dostarlimab (TSR-042, monoclonal antibody), Sasanlimab (PF-06801591, monoclonal antibody), Tislelizumab (BGB-A317, monoclonal antibody), BGB-108 (antibody), Tislelizumab (BGB-A317, antibody), Camrelizumab (INCSHR1210, SHR-1210), AMP-224, Zimberelimab (AB122, GLS-010, WBP-3055, monoclonal antibody), AK-103 (HX-008, monoclonal antibody), AK-105 (anti-PD-1 antibody), CS1003 (monoclonal antibody), HLX10 (monoclonal antibody), Retifanlimab (MGA-012, anti-PD-1 monoclonal antibody), BI-754091 (antibody), Balstilimab (AGEN2034, PD-1 antibody), toripalimab (JS-001, antibody), cetrelimab (JNJ-63723283, anti-PD-1 antibody), genolimzumab (CBT-501, anti-PD-1 antibody), LZM009 (anti-PD-1 monoclonal antibody), Prolgolimab (BCD-100, anti-PD-1 monoclonal antibody), Sym021 (antibody), ABBV-181 (antibody), BAT-1306 (antibody), JTX-4014, sintilimab (IBI-308), Tebotelimab (MGD013, PD-1/LAG-3 bispecific), MGD-019 (PD-1/CTLA4 bispecific antibody), KN-046 (PD-1/CTLA4 bispecific antibody), MEDI-5752 (CTLA4/PD-1 bispecific antibody), RO7121661 (PD-1/TIM-3 bispecific antibody), XmAb207I7 (PD-1/CTLA4 bispecific antibody), and AK-104 (CTLA4/PD-1 bispecific antibody).

In some embodiments, the third domain specifically binds to a target antigen selected from the group consisting of: wherein the target antigen is selected from the group consisting of CD19 (B-lymphocyte antigen CD19, B-Lymphocyte Surface Antigen B4, T-Cell Surface Antigen Leu-12, CVID3), PSMA (prostate specific membrane antigen), MSLN (mesothelin), BCMA (B-cell maturation antigen), DLL3 (Delta-like ligand 3), EGFR (epidermal growth factor receptor), FLT3 (FMS-like tyrosine kinase 3), CD20 (B-lymphocyte antigen CD20, MS4A1, B1, Bp35, CVID5, LEU-16, MS4A2, S7, membrane spanning 4-domains A1), CD22 (SIGLEC-2, SIGLEC2), CD25 (IL2RA, interleulin-2 receptor alpha chain), CD27 (S152, S152. LPFS2, T14, TNFRSF7, Tp55), CD30 (TNFRSF8), CD33 (Siglec-3, sialic acid binding Ig-like lectin 3, SIGLEC3, SIGLEC-3, gp67, p67), CD37 (GP52-40, TSPAN26), CD38 (cyclic ADP ribose hydrolase, ADPRC1, ADPRC 1), CD40 (Bp50, CDW40, TNFRSF5, p50), CD44 (HCAM, homing cell adhesion molecule), Pgp-1 (phagocytic glycoprotein-1), Hermes antigen, lymphocyte homing receptor, ECM-III, and HUTCH-1), CD48 (BLAST-1, B-lymphocyte activation marker, SLAMF2, signaling lymphocytic activation molecule 2), CD52 (CAMPATH-1 antigen), CD70, CD73 (NT5E, ecto-5′-nucleotidase), CD39 (ENTPD1, Ectonucleoside triphosphate diphosphohydrolase-1), CD74 (HLA class II histocompatibility antigen gamma chain, HLA-DR antigens-associated invariant chain), CD79b (immunoglobulin-associated beta), CD80 (B7-1), CD86 (B7-2), CD123 (IL3RA, interleukin-3 receptor), CD133 (PROM1), CD137 (TNFRSF9, tumor necrosis factor receptor superfamily member 9, 4-1BB, ILA, induced by lymphocyte activation), CD138 (SDC1), alpha fetoprotein (AFP), c-Met; c-Kit; CD371 (CLEC12A, C-type lectin domain family 12 member A, CLL1)); CD370 (CLEC9A, C-type lectin domain containing 9A); cadherin 3 (CDH3, p-cadherin, PCAD); carbonic anhydrase 6 (CA6); carbonic anhydrase 9 (CA9, CAIX); carcinoembryonic antigen related cell adhesion molecule 3 (CEACAM3); carcinoembryonic antigen related cell adhesion molecule 5 (CEACAM5); CD66c (CEACAM6, carcinoembryonic antigen related cell adhesion molecule 6); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EPCAM); erb-b2 receptor tyrosine kinase 2 (ERBB2, HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRa); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); CD352 (SLAMF6, SLAM family member 6); CD319 (SLAMF7, SLAM family member 7, 19A, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6, LIV1); STEAP family member 1 (STEAP1); STEAP family member 2 (STEAP2); CD134 (TNFRSF4, TNF receptor superfamily member 4, OX40); CD137L (TNFSF9, TNF superfamily member 9, 4-1BB-L); CD261 (TNFRSF10A, TNF receptor superfamily member 10a, DR4, TRAILR1); CD262 (TNFRSF10B, TNF receptor superfamily member 10b, DR5, TRAILR2); CD267 (TNFRSF13B, TNF receptor superfamily member 13B, TACI, IGAD2); CD269 (TNFRSF17, TNF receptor superfamily member 17, BCMA,); CD357 (TNFRSF18, TNF receptor superfamily member 18 GITR); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GMT; Sialyl Lewis adhesion molecule (sLe); ROR1; CD30; and Lewis Y antigen.

Provided herein is a method of improving the efficacy of a therapy comprising administering an immunomodulator to a subject, wherein the method further comprises administering to the subject a half-life extended immune cell engaging protein. In some embodiments, the immunomodulator comprises an immunostimulatory antibody agonist of a co-stimulatory receptor. In some embodiments, the immunomodulator comprises an immune checkpoint modulator. In some embodiments, the immune checkpoint modulator is an antagonist of at least one of: programmed cell death 1 (PDCD1, PD1, PD-1), CD274 (CD274, PDL1, PD-L1), PD-L2, cytotoxic T-lymphocyte associated protein 4 (CTLA4, CD152), CD276 (B7H3); V-set domain containing T cell activation inhibitor 1 (VTCN1, B7H4), CD272 (B and T lymphocyte associated (BTLA)), killer cell immunoglobulin like receptor, three Ig domains and long cytoplasmic tail 1 (KIR, CD158E1), lymphocyte activating 3 (LAG3, CD223), hepatitis A virus cellular receptor 2 (HAVCR2, TIMD3, TIM3), V-set immunoregulatory receptor (VSIR, B7H5, VISTA), T cell immunoreceptor with Ig and ITIM domains (TIGIT), programmed cell death 1 ligand 2 (PDCD1LG2, PD-L2, CD273), immunoglobulin superfamily member 11 (IGSF11, VSIG3), TNFRSF14 (HVEM, CD270), TNFSF14 (HVEML), PVR related immunoglobulin domain containing (PVRIG, CD112R), galectin 9 (LGALS9), killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1), killer cell lectin like receptor C1 (KLRC1, NKG2A, CD159A), killer cell lectin like receptor D1 (KLRD1, CD94), killer cell lectin like receptor G1 (KLRG1, CLEC15A, MAFA, 2F1), sialic acid binding Ig like lectin 7 (SIGLEC7), SIGLEC, sialic acid binding Ig like lectin 9 (SIGLEC9), CEACAM (e.g., CEACAM-1, CEACAM-3 and/or CEACAM-5), VISTA, LAIR1, CD160, 2B4, CD80, CD86, B7-H1, B7-H3 (CD276), B7-H4 (VTCN1), HVEM (TNFRSF14 or CD270), KIR, A2AR, A2BR, MHC class I, MHC class II, GAL9, adenosine, TGFR (e.g., TGFR beta), CD94/NKG2A, IDO, TDO, CD39, CD73, GARP, CD47, PVRIG, CSF1R, and NOX, or any combination thereof.

In some embodiments, the immune checkpoint modulator is an antagonist of PD-1 and is selected from the group consisting of. Pembrolizumab (humanized antibody), Pidilizumab (CT-011, monoclonal antibody, binds DLL1 and PD-1), Spartalizumab (PDR001, monoclonal antibody), Nivolumab (BMS-936558, MDX-1106, human IgG4 monoclonal antibody), MEDI0680 (AMP-514, monoclonal antibody), Cemiplimab (REGN2810, monoclonal antibody), Dostarlimab (TSR-042, monoclonal antibody), Sasanlimab (PF-06801591, monoclonal antibody), Tislelizumab (BGB-A317, monoclonal antibody), BGB-108 (antibody), Tislelizumab (BGB-A317, antibody), Camrelizumab (INCSHR1210, SHR-1210), AMP-224, Zimberelimab (AB122, GLS-010, WBP-3055, monoclonal antibody), AK-103 (HX-008, monoclonal antibody), AK-105 (anti-PD-1 antibody), CS1003 (monoclonal antibody), HLX10 (monoclonal antibody), Retifanlimab (MGA-012, anti-PD-1 monoclonal antibody), BI-754091 (antibody), Balstilimab (AGEN2034, PD-1 antibody), toripalimab (JS-001, antibody), cetrelimab (JNJ-63723283, anti-PD-1 antibody), genolimzumab (CBT-501, anti-PD-1 antibody), LZM009 (anti-PD-1 monoclonal antibody), Prolgolimab (BCD-100, anti-PD-1 monoclonal antibody), Sym021 (antibody), ABBV-181 (antibody), BAT-1306 (antibody), JTX-4014, sintilimab (IBI-308), Tebotelimab (MGD013, PD-1/LAG-3 bispecific), MGD-019 (PD-1/CTLA4 bispecific antibody), KN-046 (PD-1/CTLA4 bispecific antibody), MEDI-5752 (CTLA4/PD-1 bispecific antibody), RO7121661 (PD-1/TIM-3 bispecific antibody), XmAb207I7 (PD-1/CTLA4 bispecific antibody), and AK-104 (CTLA4/PD-1 bispecific antibody). In some embodiments, the immune checkpoint modulator is Pembrolizumab.

In some embodiments, the immune checkpoint modulator is an antibody that binds to PD-L1 and is selected from the group consisting of Atezolizumab (MPDL3280A, monoclonal antibody), Avelumab (MSB0010718C, monoclonal antibody), Durvalumab (MEDI-4736, human immunoglobulin G1 kappa (IgG1κ) monoclonal antibody), Envafolimab (KN035, single-domain PD-L1 antibody), AUNP12, CA-170 (small molecule targeting PD-L1 and VISTA), BMS-986189 (macrocyclic peptide), BMS-936559 (Anti-PD-L1 antibody), Cosibelimab (CK-301, monoclonal antibody), LY3300054 (antibody), CX-072 (antibody), CBT-502 (antibody), MSB-2311 (antibody), BGB-A333 (antibody), SHR-1316 (antibody), CS1001 (WBP3155, antibody), HLX-20 (antibody), KL-A167 (HBM 9167, antibody), STI-A1014 (antibody), STI-A1015 (IMC-001, antibody), BCD-135 (monoclonal antibody), FAZ-053 (antibody), CBT-502 (TQB2450, antibody), MDX1105-01 (antibody), FS-118 (LAG-3/PD-L1, bispecific antibody), M7824 (anti-PD-L1/TGF-0 receptor II fusion protein), CDX-527 (CD27/PD-L1 bispecific antibody), LY3415244 (TIM3/PD-L1 bispecific antibody), INBRX-105 (4-1BB/PD-L1 bispecific antibody). In some embodiments, the immune checkpoint modulator is Atezolizumab.

In some embodiments, the immune checkpoint modulator is an anti-CD39 antibody. In some embodiments, the anti-CD39 antibody is IPH5201. In some embodiments, the immune checkpoint modulator is an anti-CD73 antibody. In some embodiments, the anti-CD73 antibody is IPH5301. In some embodiments, the immunomodulator is an inhibitor of at least one of: A2AR, CD39, or CD73. In some embodiments, the inhibitor is a small molecule inhibitor.

In some embodiments, the immune checkpoint modulator comprises an immune checkpoint activator. In some embodiments, the immune checkpoint activator is an agonist of CD27, CD70, CD40, CD40LG, TNF receptor superfamily member 4 (TNFRSF4, OX40); TNF superfamily member 4 (TNFSF4, OX40L), GITR (TNF receptor superfamily member 18, TNFRSF18, CD357), TNFSF18 (GITRL), CD137 (TNFRSF9, tumor necrosis factor receptor superfamily member 9, 4-1BB, ILA, induced by lymphocyte activation), CD137L (TNFSF9), CD28, CD278 (inducible T cell co-stimulator, ICOS), inducible T cell co-stimulator ligand (ICOSLG, B7H2), CD80 (B7-1), nectin cell adhesion molecule 2 (NECTIN2, CD112), CD226 (DNAM-1), Poliovirus receptor (PVR) cell adhesion molecule (PVR, CD155), CD16, killer cell lectin like receptor K1 (KLRKI1, NKG2D, CD314), or SLAM family member 7 (SLAMF7). In some embodiments, the agonist is an antibody. In some embodiments, the subject is a human.

Provided herein is a kit comprising: (a) an immunomodulator and (b) a half-life extended immune cell engaging protein and instructions for administering (a) and (b), sequentially or concurrently, to a subject. Provided herein is a kit comprising: a combination or a composition provided herein, and instructions for administering the immunomodulator and the half-life extended immune cell engaging protein, sequentially or concurrently, to a subject.

In some embodiments, the subject has a cancer. In some embodiments, the cancer is selected from the group consisting of: mesothelioma, a prostate cancer, a breast cancer, a brain cancer, a bladder cancer, a pancreatic carcinoma, a renal cancer, a solid tumor, a liver cancer, a leiomyosarcoma, an endometrium cancer, a breast cancer, a female reproductive system cancer, a soft tissue sarcoma, a gastric cancer, a digestive/gastrointestinal cancer, a colorectal cancer, a glioblastoma multiforme, a head and neck cancer, a squamous cell carcinoma, a colon cancer, a gastric cancer, a rhabdomyosarcoma, an adrenal cancer, a lung cancer, an esophageal cancer, a colon cancer, a lung cancer, a non-small cell lung carcinoma (NSCLC), a neuroblastoma, a melanoma, glioblastoma multiforme, an ovarian cancer, an endocrine cancer, a respiratory/thoracic cancer, an anal cancer, a gastro-esophageal cancer, a thyroid cancer, a cervical cancer, an endometrial cancer, a hematological cancer, a leukemia, a lymphocytic leukemia, a multiple myeloma, a lymphoma, a Hodgkin's lymphoma, a non-Hodgkin's lymphoma, a lymphocytic leukemia, an anaplastic large-cell lymphoma (ALCL), or a myeloid leukemia. In some embodiments, the cancer is the prostate cancer. In some embodiments, the cancer is the ovarian carcinoma. In some embodiments, the cancer is the pancreatic carcinoma. In some embodiments, the cancer is the mesothelioma. In some embodiments, the cancer is the lung cancer. In some embodiments, the subject is a human.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B illustrate molecular mechanisms for the combination therapy using immunomodulators and immune cell engaging proteins. FIG. 1A illustrates administering exemplary immune cell engaging proteins (TriTAC protein) without immunomodulators, FIG. 1B illustrates administering exemplary immune cell engaging proteins (TriTAC protein) with exemplary immunomodulators (PD-1 and PD-L1 inhibitors).

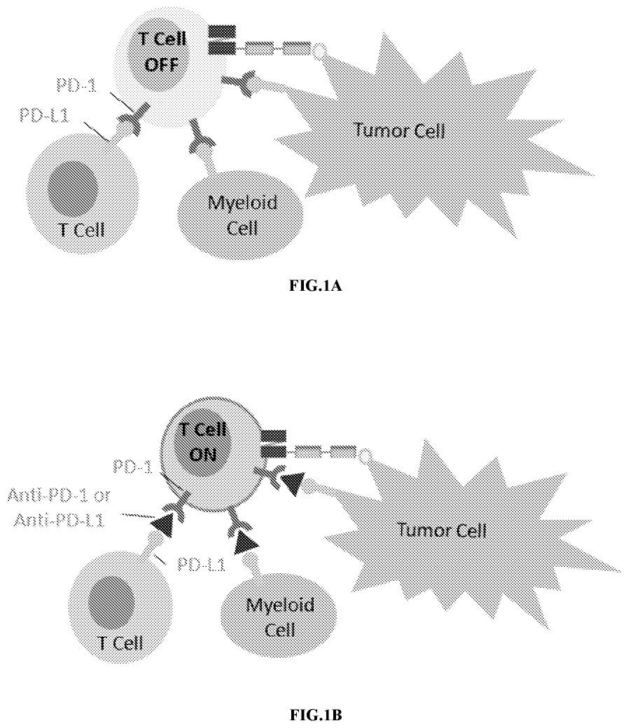

FIGS. 2A-2B illustrate FACS analysis of PD-1 and PD-L1 expression on T cells cocultured with 22Rv1 prostate cancer cells following treatment with a half-life extended prostate specific membrane antigen (PSMA) binding immune cell engaging protein. FIG. 2A illustrates PD-1 levels with donor 1764 (left) and donor 63 (right) and FIG. 2B illustrates PD-L1 levels with donor 1764 (left) and donor 63 (right).

FIG. 3 illustrates FACS analysis of PD-L1 expression on 22Rv1 prostate cancer cells following treatment with a half-life extended prostate specific membrane antigen (PSMA) binding immune cell engaging protein or IFNγ.

FIG. 4 illustrates FACS analysis of PD-L1 expression on PC3-PSMA prostate cancer cells following treatment with a half-life extended prostate specific membrane antigen (PSMA) binding immune cell engaging protein or IFNγ.

FIG. 5 illustrates 22Rv1 prostate cancer tumor model treated with a half-life extended prostate specific membrane antigen (PSMA) binding immune cell engaging protein alone or in combination with pembrolizumab or atezolizumab.

FIG. 6 illustrates PC3-PSMA prostate cancer tumor model treated with a half-life extended prostate specific membrane antigen (PSMA) binding immune cell engaging protein alone or in combination with pembrolizumab or atezolizumab.

FIGS. 7A-7B illustrate FACS analysis of PD-1 and PD-L1 expression on T cells cocultured with NCI-H292 lung cancer cells following treatment with a mesothelin (MSLN) binding immune cell engaging protein. FIG. 7A illustrates PD-1 levels with donor 1764 (left) and donor 63 (right) and FIG. 7B illustrates PD-L1 levels with donor 1764 (left) and donor 63 (right).

FIG. 8 illustrates FACS analysis of PD-L1 expression on NCI-H292 lung cancer cells following treatment with a half-life extended mesothelin (MSLN) binding immune cell engaging protein or IFNγ.

FIG. 9 illustrates FACS analysis of PD-L1 expression on OVCAR8 cancer cells following treatment with IFNγ.

FIGS. 10A-10B illustrates HCI-H292 lung cancer tumor model treated with a half-life extended mesothelin (MSLN) binding immune cell engaging protein alone or in combination with atezolizumab or pembrolizumab. The two plots represent the results of two independent experiments. FIG. 10A illustrates the results of NCI-H292-005 and FIG. 10B illustrates the results of NCI-H292-006.

FIG. 11 illustrates OVCAR8 ovarian cancer tumor model treated with a half-life extended mesothelin (MSLN) binding immune cell engaging protein alone or in combination with atezolizumab.

FIGS. 12A-12B illustrate FACS analysis of PD-1 and PD-L1 expression on T cells cocultured with SHP-77 small cell lung cancer cells following treatment with a half-life extended DLL3 binding immune cell engaging protein. FIG. 12A illustrates PD-1 levels with donor 1764 (left) and donor 63 (right) and FIG. 12B illustrates PD-L1 levels with donor 1764 (left) and donor 63 (right).

FIG. 13 illustrates FACS analysis of PD-L1 expression on SHP-77 small cell lung cancer cells following treatment with IFNγ.

FIG. 14 illustrates SHP-77 small cell lung cancer tumor model treated with a half-life extended DLL3 binding immune cell engaging protein alone or in combination with pembrolizumab or atezolizumab.

### DETAILED DESCRIPTION OF THE INVENTION

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

An “antibody” typically refers to a Y-shaped tetrameric protein comprising two heavy (H) and two light (L) polypeptide chains held together by covalent disulfide bonds and non-covalent interactions. Human light chains comprise a variable domain (VL) and a constant domain (CL) wherein the constant domain may be readily classified as kappa or lambda based on amino acid sequence and gene loci. Each heavy chain comprises one variable domain (VH) and a constant region, which in the case of IgG, IgA, and IgD, comprises three domains termed CH1, CH2, and CH3 (IgM and IgE have a fourth domain, CH4). In IgG, IgA, and IgD classes the CH1 and CH2 domains are separated by a flexible hinge region, which is a proline and cysteine rich segment of variable length (generally from about 10 to about 60 amino acids in IgG). The variable domains in both the light and heavy chains are joined to the constant domains by a “J” region of about 12 or more amino acids and the heavy chain also has a “D” region of about 10 additional amino acids. Each class of antibody further comprises inter-chain and intra-chain disulfide bonds formed by paired cysteine residues. There are two types of native disulfide bridges or bonds in immunoglobulin molecules: interchain and intrachain disulfide bonds. The location and number of interchain disulfide bonds vary according to the immunoglobulin class and species. Interchain disulfide bonds are located on the surface of the immunoglobulin, are accessible to solvent and are usually relatively easily reduced. In the human IgG1 isotype there are four interchain disulfide bonds, one from each heavy chain to the light chain and two between the heavy chains. The interchain disulfide bonds are not required for chain association. As is well known the cysteine rich IgG1 hinge region of the heavy chain has generally been held to consist of three parts: an upper hinge, a core hinge, and a lower hinge. Those skilled in the art will appreciate that that the IgG1 hinge region contain the cysteines in the heavy chain that comprise the interchain disulfide bonds (two heavy/heavy, two heavy/light), which provide structural flexibility that facilitates Fab movements. The interchain disulfide bond between the light and heavy chain of IgG1 are formed between C214 of the kappa or lambda light chain and C220 in the upper hinge region of the heavy chain. The interchain disulfide bonds between the heavy chains are at positions C226 and C229 (all numbered per the EU index according to Kabat, et al., infra.)

As used herein the term “antibody” includes polyclonal antibodies, multiclonal antibodies, monoclonal antibodies, chimeric antibodies, deimmunized, humanized and primatized antibodies, CDR grafted antibodies, human antibodies, recombinantly produced antibodies, intrabodies, multispecific antibodies, bispecific antibodies, monovalent antibodies (e.g., a monovalent IgG), multivalent antibodies, anti-idiotypic antibodies, synthetic antibodies, including muteins and variants thereof, immunospecific antibody fragments such as: hcIgG, a V-NAR, Fv, Fd, Fab, F(ab′)2, F(ab′), Fab2, Fab3 fragments, single-chain fragments (e.g., di-scFv, scFv, scFvFc, scFv-zipper, scFab), disulfide-linked Fvs (sdFv), a Fd fragment consisting of the VH and CH1 domains, linear antibodies, single domain antibodies such as nanobodies or single variable domain antibodies comprising merely one variable domain such as sdAb (VH, VL, or VHH domains), “r IgG” (“half antibody”), diabodies, single chain diabodies, tandem diabodies (Tandab's), tandem di-scFv, tandem tri-scFv, “minibodies” are in some instances exemplified by a structure which is as follows: (VH-VL-CH3)2, (scFv-CH3)2, ((scFv)2-CH3+CH3), ((scFv)2-CH3) or (scFv-CH3-scFv)2, multibodies such as triabodies or tetrabodies; and derivatives thereof including Fc fusions and other modifications, and any other immunoreactive molecule so long as it comprises a domain having a binding site for preferential association or binding with an FLT3protein. Moreover, unless dictated otherwise by contextual constraints the term further comprises all classes of antibodies (i.e. IgA, IgD, IgE, IgG, and IgM) and all subclasses (i.e., IgG1, IgG2, IgG3, IgG4, IgA1, and IgA2). Heavy-chain constant domains that correspond to the different classes of antibodies are typically denoted by the corresponding lower case Greek letter alpha, delta, epsilon, gamma, and mu, respectively. Light chains of the antibodies from any vertebrate species can be assigned to one of two clearly distinct types, called kappa (kappa) and lambda (lambda), based on the amino acid sequences of their constant domains.

As used herein, “Variable region” or “variable domain” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a (3-sheet configuration, connected by three CDRs, which form loops connecting, and in some cases forming part of, the Psheet structure. The CDRs in each chain are held together in close proximity by the FR regions and, with the CDRs from the other chain, contribute to the formation of the antigen-binding site of antibodies (see, Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. The assignment of amino acids to each domain, framework region and CDR is, in some embodiments, in accordance with one of the numbering schemes provided by Kabat et al. (1991) Sequences of Proteins of Immunological Interest (5th Ed.), US Dept. of Health and Human Services, PHS, NIH, NIH Publication no. 91-3242; Chothia et al., 1987, PMID: 3681981; Chothia et al., 1989, PMID: 2687698; MacCallum et al., 1996, PMID: 8876650; or Dubel, Ed. (2007) Handbook of Therapeutic Antibodies, 3rd Ed., Wily-VCH Verlag GmbH and Co or AbM (Oxford Molecular/MSI Pharmacopeia) unless otherwise noted.

“Variable domain residue numbering as in Kabat” or “amino acid position numbering as in Kabat,” and variations thereof, refers to the numbering system used for heavy chain variable domains or light chain variable domains of the compilation of antibodies in Kabat et al., Id. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. It is not intended that CDRs of the present disclosure necessarily correspond to the Kabat numbering convention.

The term “Framework” or “FR” residues (or regions) refer to variable domain residues other than the CDR or hypervariable region residues as herein defined. A “human consensus framework” is a framework which represents the most commonly occurring amino acid residue in a selection of human immunoglobulin VL or VH framework sequences.

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software programs such as EMBOSS MATCHER, EMBOSS WATER, EMBOSS STRETCHER, EMBOSS NEEDLE, EMBOSS LALIGN, BLAST, BLAST-2, ALIGN or MegAlign (DNASTAR) software. Those skilled in the art can determine appropriate parameters for measuring alignment, including any algorithms needed to achieve maximal alignment over the full length of the sequences being compared. Alignment for purposes of determining percent amino acid sequence identity can for example be achieved using publicly available sequence comparison computer program ALIGN-2. The source code for the ALIGN-2 sequence comparison computer program is available with user documentation in the U.S. Copyright Office, Washington D.C., 20559, where it is registered under U.S. Copyright Registration No. TXU510087. The ALIGN-2 program can be compiled for use on a UNIX operating system, such as a digital UNIX V4.0D. All sequence comparison parameters are set by the ALIGN-2 program and do not vary.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time-1 and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e., after drug absorption and distribution are complete). The half-time for drug elimination can be accurately determined from such a graph.

As used herein, the term “binding affinity” refers to the affinity of the proteins described in the disclosure to their binding targets, and is expressed numerically using “Kd” values. If two or more proteins are indicated to have comparable binding affinities towards their binding targets, then the Kd values for binding of the respective proteins towards their binding targets, are within ±2-fold of each other. If two or more proteins are indicated to have comparable binding affinities towards single binding target, then the Kd values for binding of the respective proteins towards said single binding target, are within ±2-fold of each other. If a protein is indicated to bind two or more targets with comparable binding affinities, then the Kd values for binding of said protein to the two or more targets are within ±2-fold of each other. In general, a higher Kd value corresponds to a weaker binding. In some embodiments, the “Kd” is measured by a radiolabeled antigen binding assay (RIA) or surface plasmon resonance assays using a BIACORE™-2000 or a BIACORE™-3000 (BIAcore, Inc., Piscataway, N.J.). In certain embodiments, an “on-rate” or “rate of association” or “association rate” or “kon” and an “off-rate” or “rate of dissociation” or “dissociation rate” or “koff” are also determined with the surface plasmon resonance technique using a BIAcore™-2000 or a BIAcore™-3000 (BIAcore, Inc., Piscataway, N.J.). In additional embodiments, the “Kd”, “kon”, and “koff” are measured using the OCTET® Systems (Pall Life Sciences). In an exemplary method for measuring binding affinity using the OCTET® Systems, the ligand, e.g., biotinylated human or cynomolgus FLT3 in case of an immune cell engaging protein comprising a FLT3 binding single domain antibody, is immobilized on the OCTET® streptavidin capillary sensor tip surface which streptavidin tips are then activated according to manufacturer's instructions using about 20-50 μg/ml human or cynomolgus FLT3 protein. A solution of PBS/Casein is also introduced as a blocking agent. For association kinetic measurements, FLT3 binding protein variants are introduced at a concentration ranging from about 10 ng/mL to about 100 μg/mL, about 50 ng/mL to about 5 μg/mL, or about 2 ng/mL to about 20 μg/mL. In some embodiments, the FLT3 binding single domain proteins are used at a concentration ranging from about 2 ng/mL to about 20 μg/mL. Complete dissociation is observed in case of the negative control, assay buffer without the binding proteins. The kinetic parameters of the binding reactions are then determined using an appropriate tool, e.g., ForteBio software.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

Generally, it should be noted that the term single domain antibody as used herein in its broadest sense is not limited to a specific biological source or to a specific method of preparation. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, bovine. For example, in some embodiments, the single domain antibodies of the disclosure are obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a species of mammal, such as from a human being, or by expression of a nucleic acid encoding such a camelized VH domain; (5) by “camelization” of a “domain antibody” or “Dab,” or by expression of a nucleic acid encoding such a camelized VH domain; (6) by using synthetic or semi-synthetic techniques for preparing proteins, polypeptides or other amino acid sequences; (7) by preparing a nucleic acid encoding a single domain antibody using techniques for nucleic acid synthesis known in the field, followed by expression of the nucleic acid thus obtained; and/or (8) by any combination of one or more of the foregoing.

In some embodiments, an immune cell engaging protein as described herein comprises an antibody, e.g., a single domain antibody targeting an antigen, e.g., PSMA, MSLN, BCMA, DLL3, EGFR, EpCAM, FLT3, comprising an amino acid sequence that corresponds to the amino acid sequence of a naturally occurring VHH domain, but that has been “humanized”, e.g., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above). This can be performed in a manner known in the field, which will be clear to the skilled person, for example on the basis of the further description herein. Again, it should be noted that such humanized single domain antibodies of the disclosure are obtained in any suitable manner known per se (e.g., as indicated under points (1)-(8) above) and thus are not strictly limited to polypeptides that have been obtained using a polypeptide that comprises a naturally occurring VHH domain as a starting material. In some additional embodiments, a single domain antibody, as described herein, comprises a single domain antibody with an amino acid sequence that corresponds to the amino acid sequence of a naturally occurring VH domain, but that has been “camelized”, i.e., by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized anti-MSLN single domain antibodies of the disclosure, in certain embodiments, are obtained in any suitable manner known in the field (i.e., as indicated under points (1)-(8) above) and thus are not strictly limited to polypeptides that have been obtained using a polypeptide that comprises a naturally occurring VH domain as a starting material. For example, as further described herein, both “humanization” and “camelization” is performed by providing a nucleotide sequence that encodes a naturally occurring VHH domain or VH domain, respectively, and then changing, one or more codons in said nucleotide sequence in such a way that the new nucleotide sequence encodes a “humanized” or “camelized” single domain antibody, respectively. This nucleic acid can then be expressed, so as to provide a desired single domain antibody of the disclosure. Alternatively, in other embodiments, based on the amino acid sequence of a naturally occurring VHH domain or VH domain, respectively, the amino acid sequence of the desired humanized or camelized single domain antibody of the disclosure, respectively, are designed and then synthesized de novo using known techniques for peptide synthesis. In some embodiments, based on the amino acid sequence or nucleotide sequence of a naturally occurring VHH domain or VH domain, respectively, a nucleotide sequence encoding the desired humanized or camelized single domain antibody of the disclosure, respectively, is designed and then synthesized de novo using known techniques for nucleic acid synthesis, after which the nucleic acid thus obtained is expressed in using known expression techniques, so as to provide the desired single domain antibody of the disclosure.

The terms “patient” or “subject” refers to any single subject for which therapy is desired or that is participating in a clinical trial, epidemiological study or used as a control, including humans and mammalian veterinary patients such as cattle, horses, dogs, and cats.

### Immune Cell Engaging Proteins

Described herein, in some embodiments, is a combination comprising an immunomodulator and an immune cell engaging protein. In some embodiments, the immune cell engaging protein is a bi-specific protein, a tri-specific protein or a multi-specific protein. In some embodiments, the immune cell engaging protein is a half-life extended protein. In some embodiments, the immune cell engaging protein comprises a domain that binds to a bulk serum protein, e.g., a serum albumin. In some embodiments, the serum albumin is human serum albumin (HSA).

In some embodiments, the immune cell engaging protein comprises an immune cell engaging domain. In some embodiments “an immune cell engaging domain” as used herein refers to one or more binding specificities that bind and/or activate an immune cell, e.g., a cell involved in an immune response. In some embodiments, the immune cell is selected from an NK cell, a B cell, a dendritic cell, a macrophage cell. The immune cell engaging domain, in some embodiments, is an antibody or an antigen binding fragment thereof, a receptor molecule (e.g., a full length receptor, receptor fragment, or fusion thereof (e.g., a receptor-Fc fusion)), or a ligand molecule (e.g., a full length ligand, ligand fragment, or fusion thereof (e.g., a ligand-Fc fusion)) that binds to the immune cell antigen (e.g., the NK cell antigen, the B cell antigen, the dendritic cell antigen, and/or the macrophage cell antigen). In embodiments, the immune cell engaging domain specifically binds to the target immune cell, e.g., binds preferentially to the target immune cell. For example, in some embodiments the immune cell engaging domain is an antibody or an antigen binding fragment thereof that binds to the immune cell antigen (e.g., the NK cell antigen, the B cell antigen, the dendritic cell antigen, and/or the macrophage cell antigen) with a dissociation constant of less than about 10 nM.

In some embodiments, the immune cell engaging domain comprises a natural killer (NK) cell engaging domain, a T cell engaging domain, a B cell engaging domain, a dendritic cell engaging domain, a macrophage cell engaging domain, or a combination thereof. In some embodiments, the immune cell engaging protein comprises a T-cell engaging domain. In some embodiments, the T cell engaging domain binds a CD3 molecule. In some embodiments, the CD3 molecule is at least one of: a CD3γ molecule, a CD3δ molecule, or a CD3ε molecule.

In some embodiments, the immune cell engaging protein comprises an antigen binding domain. In some examples, the antigen binding domain includes antibodies, single chain antibodies, Fabs, Fv, T-cell receptor binding domains, ligand binding domains, receptor binding domains, domain antibodies, single domain antibodies, minibodies, nanobodies, peptibodies, or various other antibody mimics (such as affimers, affitins, alphabodies, atrimers, CTLA4-based molecules, adnectins, anticalins, Kunitz domain-based proteins, avimers, knottins, fynomers, darpins, affibodies, affilins, monobodies and armadillo repeat protein-based proteins).

In some embodiments, the antigen is a tumor antigen, In some embodiments, the antigen is selected from the group consisting of: CD19 (B-lymphocyte antigen CD19, B-Lymphocyte Surface Antigen B4, T-Cell Surface Antigen Leu-12, CVID3), PSMA (prostate specific membrane antigen), MSLN (mesothelin), BCMA (B-cell maturation antigen), DLL3 (Delta-like ligand 3), EGFR (epidermal growth factor receptor), FLT3 (FMS-like tyrosine kinase 3), CD20 (B-lymphocyte antigen CD20, MS4A1, B1, Bp35, CVID5, LEU-16, MS4A2, S7, membrane spanning 4-domains A1), CD22 (SIGLEC-2, SIGLEC2), CD25 (IL2RA, interleulin-2 receptor alpha chain), CD27 (S152, S152. LPFS2, T14, TNFRSF7, Tp55), CD30 (TNFRSF8), CD33 (Siglec-3, sialic acid binding Ig-like lectin 3, SIGLEC3, SIGLEC-3, gp67, p67), CD37 (GP52-40, TSPAN26), CD38 (cyclic ADP ribose hydrolase, ADPRC1, ADPRC 1), CD40 (Bp50, CDW40, TNFRSF5, p50), CD44 (HCAM, homing cell adhesion molecule), Pgp-1 (phagocytic glycoprotein-1), Hermes antigen, lymphocyte homing receptor, ECM-III, and HUTCH-1), CD48 (BLAST-1, B-lymphocyte activation marker, SLAMF2, signaling lymphocytic activation molecule 2), CD52 (CAMPATH-1 antigen), CD70, CD73 (NT5E, ecto-5′-nucleotidase), CD39 (ENTPD1, Ectonucleoside triphosphate diphosphohydrolase-1), CD74 (HLA class II histocompatibility antigen gamma chain, HLA-DR antigens-associated invariant chain), CD79b (immunoglobulin-associated beta), CD80 (B7-1), CD86 (B7-2), CD123 (IL3RA, interleukin-3 receptor), CD133 (PROM1), CD137 (TNFRSF9, tumor necrosis factor receptor superfamily member 9, 4-1BB, ILA, induced by lymphocyte activation), CD138 (SDC1), alpha fetoprotein (AFP), c-Met; c-Kit; CD371 (CLEC12A, C-type lectin domain family 12 member A, CLL1)); CD370 (CLEC9A, C-type lectin domain containing 9A); cadherin 3 (CDH3, p-cadherin, PCAD); carbonic anhydrase 6 (CA6); carbonic anhydrase 9 (CA9, CAIX); carcinoembryonic antigen related cell adhesion molecule 3 (CEACAM3); carcinoembryonic antigen related cell adhesion molecule 5 (CEACAM5); CD66c (CEACAM6, carcinoembryonic antigen related cell adhesion molecule 6); chorionic somatomammotropin hormone 1 (CSH1, CS1); coagulation factor III, tissue factor (F3, TF); collectin subfamily member 10 (COLEC10); delta like canonical Notch ligand 3 (DLL3); ectonucleotide pyrophosphatase/phosphodiesterase 3 (ENPP3); ephrin A1 (EFNA1); epidermal growth factor receptor (EGFR); EGFR variant III (EGFRvIII); EPH receptor A2 (EPHA2); epithelial cell adhesion molecule (EpCAM); erb-b2 receptor tyrosine kinase 2 (ERBB2, HER2); fibroblast activation protein alpha (FAP); fibroblast growth factor receptor 2 (FGFR2); fibroblast growth factor receptor 3 (FGFR3); folate hydrolase 1 (FOLH1, PSMA); folate receptor 1 (FOLR1, FRa); GD2 ganglioside; glycoprotein NMB (GPNMB, osteoactivin); guanylate cyclase 2C (GUCY2C, GCC); human papillomavirus (HPV) E6; HPV E7; major histocompatibility complex (MHC) class I-presented neoantigens, major histocompatibility complex (MHC) class II-presented neoantigens, major histocompatibility complex, class I, E (HLA-E); major histocompatibility complex, class I, F (HLA-F); major histocompatibility complex, class I, G (HLA-G, MHC-G); integrin subunit beta 7 (ITGB7); leukocyte immunoglobulin like receptor B1 (LILRB1, ILT2); leukocyte immunoglobulin like receptor B2 (LILRB2, ILT4); LY6/PLAUR domain containing 3 (LYPD3, C4.4A); glypican 3 (GPC3); KRAS proto-oncogene, GTPase (KRAS); MAGE family member A1 (MAGEA1); MAGE family member A3 (MAGEA3); MAGE family member A4 (MAGEA4); MAGE family member A11 (MAGEA11); MAGE family member C1 (MAGEC1); MAGE family member C2 (MAGEC2); MAGE family member D1 (MAGED1); MAGE family member D2 (MAGED2); mesothelin (MSLN); mucin 1 (MUC1) and splice variants thereof (e.g., MUC1/C, D, and Z); mucin 16 (MUC16); necdin (NDN); nectin cell adhesion molecule 4 (NECTIN4); SLIT and NTRK like family member 6 (SLITRK6); promyelocytic leukemia (PML, TRIM19); protein tyrosine kinase 7 (inactive) (PTK7); CD352 (SLAMF6, SLAM family member 6); CD319 (SLAMF7, SLAM family member 7, 19A, CRACC, CS1); sialic acid binding Ig like lectin 7 (SIGLEC7); sialic acid binding Ig like lectin 9 (SIGLEC9); solute carrier family 34 (sodium phosphate), member 2 (SLC34A2); solute carrier family 39 member 6 (SLC39A6, LIV1); STEAP family member 1 (STEAP1); STEAP family member 2 (STEAP2); CD134 (TNFRSF4, TNF receptor superfamily member 4, OX40); CD137L (TNFSF9, TNF superfamily member 9, 4-1BB-L); CD261 (TNFRSF10A, TNF receptor superfamily member 10a, DR4, TRAILR1); CD262 (TNFRSF10B, TNF receptor superfamily member 10b, DR5, TRAILR2); CD267 (TNFRSF13B, TNF receptor superfamily member 13B, TACI, IGAD2); CD269 (TNFRSF17, TNF receptor superfamily member 17, BCMA,); CD357 (TNFRSF18, TNF receptor superfamily member 18 GITR); transferrin (TF); transforming growth factor beta 1 (TGFB1); trophoblast glycoprotein (TPBG, 5T4); trophinin (TRO, MAGED3); tumor associated calcium signal transducer 2 (TACSTD2, TROP2, EGP1); Fucosyl GM1; sialyl Lewis adhesion molecule (sLe); ROR1, CD30, and Lewis Y antigen. In some embodiments, the immune cell engaging protein specifically binds to PSMA, MSLN, BCMA, DLL3, EGFR, FLT3, or EpCAM.

Describe herein is an immune cell engaging protein comprises a first domain (A), which is a T cell engaging domain and specifically binds to human CD3, a second domain (B) which specifically binds to human serum albumin (HSA), and a third domain (C) which specifically binds to a target antigen. In some embodiments, the immune cell engaging protein's domains are linked in one of the following orders: H₂N-(C)-(B)-(A)-COOH, H₂N-(A)-(B)-(C)-COOH, H₂N-(B)-(A)-(C)-COOH, H₂N-(C)-(A)-(B)-COOH, H₂N-(A)-(C)-(B)-COOH, H₂N-(B)-(C)-(A)-COOH, or by linkers L1 and L2 in one of the following orders: H₂N-(C)-L1-(B)-L2(A)-COOH, H₂N-(A)-L1-(B)-L2-(C)-COOH, H₂N-(B)-L1-(A)-L2-(C)-COOH, H₂N-(C)-L1-(A)-L2-(B)-COOH, H₂N-(A)-L1-(C)-L2(B)-COOH, H₂N-(B)-L1-(C)-L2-(A)-COOH. In some embodiments, the linkers L1 and L2 are each independently selected from an amino acid sequence of SEQ ID NOS: 3190-3200.

In some embodiments, the immune cell engaging protein described herein comprise a polypeptide having a sequence described in SEQ ID NOS: 3218-3462 and subsequences thereof. In some embodiments, the immune cell engaging protein comprises a polypeptide having at least 70%-95% or more homology to a sequence described in SEQ ID NO: 3218-3462. In some embodiments, the immune cell engaging protein comprises a polypeptide having at least 70%, 75%, 80%, 85%, 90%, 95%, or more homology to a sequence described in SEQ ID NO: 3218-3462. In some embodiments, the immune cell engaging protein has a sequence comprising at least a portion of a sequence described in SEQ ID NO: 3218-3462. In some embodiments, the immune cell engaging protein comprises a polypeptide comprising one or more of the sequences described in SEQ ID NO: 3218-3462.

In some embodiments, the immune cell engaging protein described herein comprise a polypeptide having a sequence described in SEQ ID NOS: 3255, 3340, 3376, and 3462 and subsequences thereof. In some embodiments, the immune cell engaging protein comprises a polypeptide having at least 70%-95% or more homology to a sequence described in SEQ ID NOS: 3255, 3340, 3376, and 3462. In some embodiments, the immune cell engaging protein comprises a polypeptide having at least 70%, 75%, 80%, 85%, 90%, 95%, or more homology to a sequence described in SEQ ID NOS: 3255, 3340, 3376, and 3462. In some embodiments, the immune cell engaging protein has a sequence comprising at least a portion of a sequence described in SEQ ID NOS: 3255, 3340, 3376, and 3462. In some embodiments, the immune cell engaging protein comprises a polypeptide comprising one or more of the sequences described in SEQ ID NOS: 3255, 3340, 3376, and 3462.

### Prostate Specific Membrane Antigen (PSMA) Binding Proteins

Described herein are immune cell engaging proteins that comprise an PSMA binding domain, pharmaceutical compositions thereof, as well as nucleic acids, recombinant expression vectors and host cells for making such proteins thereof. Also provided are methods of using the disclosed proteins comprising an PSMA binding domain of this disclosure, in the prevention, and/or treatment of diseases, conditions and disorders.

PSMA is a 100 kD Type II membrane glycoprotein expressed in prostate tissues having sequence identity with the transferrin receptor with NAALADase activity. PSMA is expressed in increased amounts in prostate cancer, and elevated levels of PSMA are also detectable in the sera of these patients. PSMA expression increases with disease progression, becoming highest in metastatic, hormone-refractory disease for which there is no present therapy.

The design of the PSMA targeting immune cell engaging proteins described herein allows the binding domain to PSMA to be flexible in that the binding domain to PSMA can be any type of binding domain, including but not limited to, domains from a monoclonal antibody, a polyclonal antibody, a recombinant antibody, a human antibody, a humanized antibody. In some embodiments, the binding domain to PSMA is a single chain variable fragments (scFv), single-domain antibody such as a heavy chain variable domain (VH), a light chain variable domain (VL) and a variable domain (VHH) of camelid derived single domain antibody. In other embodiments, the binding domain to PSMA is a non-Ig binding domain, i.e., antibody mimetic, such as anticalins, affilins, affibody molecules, AFFIMERs®, affitins, alphabodies, avimers, DARPins, fynomers, kunitz domain peptides, and monobodies. In further embodiments, the binding domain to PSMA is a ligand or peptide that binds to or associates with PSMA. In yet further embodiments, the binding domain to PSMA is a knottin. In yet further embodiments, the binding domain to PSMA is a small molecular entity.

In some embodiments, the PSMA binding domain comprises the following formula: f1-r1-f2-r2-f3-r3-f4, wherein r1, r2, and r3 are complementarity determining regions CDR1, CDR2, and CDR3, respectively, and f1, f2, f3, and f4 are framework residues, and wherein r1 comprises SEQ ID NO: 462, SEQ ID NO: 463, SEQ ID NO: 464, or SEQ ID NO: 465, r2 comprises SEQ ID NO: 466, SEQ ID NO: 467, SEQ ID NO: 468, SEQ ID NO: 469, SEQ ID NO: 470, SEQ ID NO: 471, SEQ ID NO: 472, or SEQ ID NO: 473, and r3 comprises SEQ ID NO: 474, or SEQ ID NO: 475.

In some embodiments, PSMA binding domains described herein comprise a polypeptide having a sequence described in SEQ ID NO: 462-489 and subsequences thereof. In some embodiments, the HSA binding domain comprises a polypeptide having at least 70%-95% or more homology to a sequence described in SEQ ID NO: 462-489. In some embodiments, the HSA binding domain comprises a polypeptide having at least 70%, 75%, 80%, 85%, 90%, 95%, or more homology to a sequence described in SEQ ID NO: 462-489. In some embodiments, the HSA binding domain has a sequence comprising at least a portion of a sequence described in SEQ ID NO: 462-489. In some embodiments, the HSA binding domain comprises a polypeptide comprising one or more of the sequences described in SEQ ID NO: 462-489.

In some embodiments, PSMA binding domains described herein comprise a single domain antibody with a CDR1 comprising SEQ ID NO: 462-465. In some embodiments, PSMA binding domains described herein comprise a single domain antibody with a CDR2 comprising SEQ ID NO: 466-473. In some embodiments, PSMA binding domains described herein comprise a single domain antibody with a CDR3 comprising SE ID NO: 474 and 475.

### Mesothelin (MSLN) Binding Proteins

MSLN is a GPI-linked membrane bound tumor antigen. MSLN is overexpressed ovarian, pancreatic, lung and triple-negative breast cancers and mesothelioma. Normal tissue expression of MSLN is restricted to single-cell, mesothelial layers lining the pleural, pericardial, and peritoneal cavities. Overexpression of MSLN is associated with poor prognosis in lung adenocarcinoma and triple-negative breast cancer. MSLN has been used as cancer antigen for numerous modalities, including immunotoxins, vaccines, antibody drug conjugates and CAR-T cells. Early signs of clinical efficacy have validated MSLN as a target, but therapies with improved efficacy are needed to treat MSLN-expressing cancers.

Mesothelin is a glycoprotein present on the surface of cells of the mesothelial lining of the peritoneal, pleural and pericardial body cavities. The mesothelin gene (MSLN) encodes a 71 kD precursor protein that is processed to a 40 kD protein termed mesothelin, which is a glycosyl-phosphatidylinositol-anchored glycoprotein present on the cell surface (Chang, et al, Proc Natl Acad Sci USA (1996) 93:136-40). The mesothelin cDNA was cloned from a library prepared from the HPC-Y5 cell line (Kojima et al. (1995) J. Biol. Chem. 270:21984-21990). The cDNA also was cloned using the monoclonal antibody K1, which recognizes mesotheliomas (Chang and Pastan (1996) Proc. Natl. Acad. Sci. USA 93:136-40). Mesothelin is a differentiation antigen whose expression in normal human tissues is limited to mesothelial cells lining the body cavity, such as the pleura, pericardium and peritoneum. Mesothelin is also highly expressed in several different human cancers, including mesotheliomas, pancreatic adenocarcinomas, ovarian cancers, stomach and lung adenocarcinomas. (Hassan, et al., Eur J Cancer (2008) 44:46-53) (Ordonez, Am J Surg Pathol (2003) 27:1418-28; Ho, et al., Clin Cancer Res (2007) 13:1571-5). Mesothelin is overexpressed in a vast majority of primary pancreatic adenocarcinomas with rare and weak expression seen in benign pancreatic tissue. Argani P, et al. Clin Cancer Res. 2001; 7(12):3862-3868. Epithelial malignant pleural mesothelioma (MPM) universally expresses mesothelin while sarcomatoid MPM likely does not express mesothelin. Most serous epithelial ovarian carcinomas, and the related primary peritoneal carcinomas, express mesothelin.

Mesothelin can also be used a marker for diagnosis and prognosis of certain types of cancer because trace amounts of mesothelin can be detected in the blood of some patients with mesothelin-positive cancers (Cristaudo et al., Clin. Cancer Res. 13:5076-5081, 2007). It has been reported that mesothelin may be released into serum through deletion at its carboxyl terminus or by proteolytic cleavage from its membrane bound form (Hassan et al., Clin. Cancer Res. 10:3937-3942, 2004). An increase in the soluble form of mesothelin was detectable several years before malignant mesotheliomas occurred among workers exposed to asbestos (Creaney and Robinson, Hematol. Oncol. Clin. North Am. 19:1025-1040, 2005). Furthermore, patients with ovarian, pancreatic, and lung cancers also have elevated soluble mesothelin in serum (Cristaudo et al., Clin. Cancer Res. 13:5076-5081, 2007; Hassan et al., Clin. Cancer Res. 12:447-453, 2006; Croso et al., Cancer Detect. Prev. 30:180-187, 2006). Accordingly, mesothelin is an appropriate target for methods of disease prevention or treatment and there is a need for effective antibodies specific for mesothelin.

It has been shown that cell surface mature mesothelin comprises three distinct domains, namely Regions I (comprising residues 296-390), II (comprising residues 391-486), and III (comprising residue 487-598). (Tang et al., A human single-domain antibody elicits potent antitumor activity by targeting an epitope in mesothelin close to the cancer cell surface, Mol. Can. Therapeutics, 12(4): 416-426, 2013). The first antibodies generated against mesothelin for therapeutic intervention were designed to interfere with the interaction between mesothelin and CA-125. Phage display identified the Fv SS, which was affinity optimized and used to generate a recombinant immunotoxin targeting mesothelin, SS1P. The MORAb-009 antibody amatuximab, which also uses SS1, recognizes a non-linear epitope in the amino terminal 64 amino acids of mesothelin, within region I. The SS1 Fv was also used to generate chimeric antigen receptor-engineered T cells. Recently, new anti-mesothelin antibodies have been reported that recognize other regions of the mesothelin protein.

There is still a need for having available further options for the treatment of solid tumor diseases related to the overexpression of mesothelin, such as ovarian cancer, pancreatic cancer, mesothelioma, lung cancer, gastric cancer and triple negative breast cancer. The present disclosure provides, in certain embodiments, MSLN targeting immune cell engaging proteins containing binding domains which specifically bind to MSLN on the surface of tumor target cells.

The design of the MSLN targeting immune cell engaging proteins described herein allows the binding domain to MSLN to be flexible in that the binding domain to MSLN can be any type of binding domain, including but not limited to, domains from a monoclonal antibody, a polyclonal antibody, a recombinant antibody, a human antibody, a humanized antibody. In some embodiments, the binding domain to MSLN is a single chain variable fragments (scFv), single-domain antibody such as a heavy chain variable domain (VH), a light chain variable domain (VL) and a variable domain (VHH) of camelid derived single domain antibody. In other embodiments, the binding domain to MSLN is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to MSLN is a ligand or peptide that binds to or associates with MSLN. In yet further embodiments, the binding domain to MSLN is a knottin. In yet further embodiments, the binding domain to MSLN is a small molecular entity.

In some embodiments, the MSLN binding domain binds to a protein comprising the sequence of SEQ ID NO: 3204. In some embodiments, the MSLN binding domain binds to a protein comprising a truncated sequence compared to SEQ ID NO: 3204.

In some embodiments, the MSLN binding domains disclosed herein recognize full-length mesothelin. In certain instances, the MSLN binding domains disclosed herein recognize an epitope in region I (comprising amino acid residues 296-390 of SEQ ID NO: 3204), region II (comprising amino acid residue 391-486 of SEQ ID NO: 3204), or region III (comprising amino acid residues 487-598 of SEQ ID NO: 3204) of mesothelin. It is contemplated that the MSLN binding domains of the present disclosure may, in some embodiments, recognize and bind to epitopes that are located outside regions I, II, or III of mesothelin. In yet other embodiments are disclosed MSLN binding domains that recognize and bind to an epitope different than the MORAb-009 antibody.

In some embodiments, the MSLN binding domain is an anti-MSLN antibody or an antibody variant. As used herein, the term “antibody variant” refers to variants and derivatives of an antibody described herein. In certain embodiments, amino acid sequence variants of the anti-MSLN antibodies described herein are contemplated. For example, in certain embodiments amino acid sequence variants of anti-MSLN antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant anti-MSLN antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the MSLN binding domain of the MSLN targeting immune cell engaging protein is a single domain antibody such as a heavy chain variable domain (VH), a variable domain (VHH) of a llama derived sdAb, a peptide, a ligand or a small molecule entity specific for mesothelin. In some embodiments, the mesothelin binding domain of the MSLN targeting immune cell engaging protein described herein is any domain that binds to mesothelin including but not limited to domains from a monoclonal antibody, a polyclonal antibody, a recombinant antibody, a human antibody, a humanized antibody. In certain embodiments, the MSLN binding domain is a single-domain antibody. In other embodiments, the MSLN binding domain is a peptide. In further embodiments, the MSLN binding domain is a small molecule.

In one embodiment, a single domain antibody corresponds to the VHH domains of naturally occurring heavy chain antibodies directed against MSLN. As further described herein, such VHH sequences can generally be generated or obtained by suitably immunizing a species of Llama with MSLN, (i.e., so as to raise an immune response and/or heavy chain antibodies directed against MSLN), by obtaining a suitable biological sample from said Llama (such as a blood sample, serum sample or sample of B-cells), and by generating VHH sequences directed against MSLN, starting from said sample, using any suitable technique known in the field.

In another embodiment, such naturally occurring VHH domains against MSLN, are obtained from naïve libraries of Camelid VHH sequences, for example by screening such a library using MSLN, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against MSLN, involves suitably immunizing a transgenic mammal that is capable of expressing heavy chain antibodies (i.e., so as to raise an immune response and/or heavy chain antibodies directed against MSLN), obtaining a suitable biological sample from said transgenic mammal (such as a blood sample, serum sample or sample of B-cells), and then generating VHH sequences directed against MSLN, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-MSLN single domain antibody of the MSLN targeting immune cell engaging protein comprises a single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining the anti-MSLN single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-MSLN single domain antibody of the disclosure or a nucleotide sequence or nucleic acid encoding the same.

In some embodiments, the MSLN binding domain is an anti-MSLN specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the MSLN binding domain comprises any domain that binds to MSLN including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the MSLN binding domain is a single domain antibody. In some embodiments, the anti-MSLN single domain antibody comprises heavy chain variable complementarity determining regions (CDR), CDR1, CDR2, and CDR3.

In some embodiments, the MSLN binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the MSLN binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the MSLN binding domain comprises an amino acid sequence selected from the group consisting of SEQ ID NOS: 607-650.

In some embodiments, the CDR1 comprises the amino acid sequence as set forth in SEQ ID NO: 490 or a variant having one, two, three, four, five, six, seven, eight, nine, or ten amino acid substitutions in SEQ ID NO: 490. In some embodiments, the CDR2 comprises a sequence as set forth in SEQ ID NO: 3505 or a variant having one, two, three, four, five, six, seven, eight, nine, or ten amino acid substitutions in SEQ ID NO: 3505. In some embodiments, the CDR3 comprises a sequence as set forth in SEQ ID NO: 3506 or a variant having one, two, three, four, five, six, seven, eight, nine, or ten amino acid substitutions in SEQ ID NO: 3506.

In some embodiments, the CDR1 comprises the amino acid sequence as set forth in SEQ ID NO: 518 or a variant having one, two, three, four, five, six, seven, eight, nine, or ten amino acid substitutions in SEQ ID NO: 518. In some embodiments, the CDR2 comprises a sequence as set forth in SEQ ID NO: 3507 or a variant having one, two, three, four, five, six, seven, eight, nine, or ten amino acid substitutions in SEQ ID NO: 3507. In some embodiments, the CDR3 comprises a sequence as set forth in SEQ ID NO: 3508 or a variant having one, two, three, four, five, six, seven, eight, nine, or ten amino acid substitutions in SEQ ID NO: 3508.

In some embodiments, the CDR1 comprises the amino acid sequence as set forth in any one of SEQ ID NOS: 490-528 or a variant having one, two, three, four, five, six, seven, eight, nine, or ten amino acid substitutions in any one of SEQ ID NOS: 490-528. In some embodiments, the CDR2 comprises a sequence as set forth in any one of SEQ ID NOS: 529-567 or a variant having one, two, three, four, five, six, seven, eight, nine, or ten amino acid substitutions in any one of SEQ ID NOS: 529-567. In some embodiments, the CDR3 comprises a sequence as set forth in any one of SEQ ID NOS: 568-606 or a variant having one, two, three, four, five, six, seven, eight, nine, or ten amino acid substitutions in any one of SEQ ID NOS: 568-606.

In various embodiments, the MSLN binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 607-650.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 490, SEQ ID NO: 518, or any one of SEQ ID NOS: 490-528.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 3505, SEQ ID NO: 3507, or any one of SEQ ID NOS: 529-567.

In various embodiments, a complementarity determining region of the MSLN binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NO: 3506, SEQ ID NO: 3508, or any one of SEQ ID NOS: 568-606.

In some embodiments, the MSLN binding protein, according to any one of the above embodiments, is a single domain antibody comprising the sequence of SEQ ID NO: 607. In some embodiments, the MSLN binding protein, according to any one of the above embodiments, is a single domain antibody comprising the sequence of SEQ ID NO: 608. In some embodiments, the MSLN binding protein, according to any one of the above embodiments, is a single domain antibody comprising the sequence of SEQ ID NO: 609. In some embodiments, the MSLN binding protein, according to any one of the above embodiments, is a single domain antibody comprising the sequence of SEQ ID NO: 610. In some embodiments, the MSLN binding protein, according to any one of the above embodiments, is a single domain antibody comprising the sequence of SEQ ID NO: 611. In some embodiments, the MSLN binding protein, according to any one of the above embodiments, is a single domain antibody comprising the sequence of SEQ ID NO: 612. In some embodiments, the MSLN binding protein, according to any one of the above embodiments, is a single domain antibody comprising the sequence of SEQ ID NO: 613. In some embodiments, the MSLN binding protein, according to any one of the above embodiments, is a single domain antibody comprising the sequence of SEQ ID NO: 614. In some embodiments, the MSLN binding protein, according to any one of the above embodiments, is a single domain antibody comprising the sequence of SEQ ID NO: 615. In some embodiments, the MSLN binding protein, according to any one of the above embodiments, is a single domain antibody comprising the sequence of SEQ ID NO: 616. In some embodiments, the MSLN binding protein, according to any one of the above embodiments, is a single domain antibody comprising the sequence of SEQ ID NO: 617. In some embodiments, the MSLN binding protein, according to any one of the above embodiments, is a single domain antibody comprising the sequence of SEQ ID NO: 618. In some embodiments, the MSLN binding protein, according to any one of the above embodiments, is a single domain antibody comprising the sequence of SEQ ID NO: 619. In some embodiments, the MSLN binding protein, according to any one of the above embodiments, is a single domain antibody comprising the sequence of SEQ ID NO: 620. In some embodiments, the MSLN binding protein, according to any one of the above embodiments, is a single domain antibody comprising the sequence of SEQ ID NO: 621. In some embodiments, the MSLN binding protein, according to any one of the above embodiments, is a single domain antibody comprising the sequence of SEQ ID NO: 622. In some embodiments, the MSLN binding protein, according to any one of the above embodiments, is a single domain antibody comprising the sequence of SEQ ID NO: 623. In some embodiments, the MSLN binding protein, according to any one of the above embodiments, is a single domain antibody comprising the sequence of SEQ ID NO: 624. In some embodiments, the MSLN binding protein, according to any one of the above embodiments, is a single domain antibody comprising the sequence of SEQ ID NO: 625. In some embodiments, the MSLN binding protein, according to any one of the above embodiments, is a single domain antibody comprising the sequence of SEQ ID NO: 626. In some embodiments, the MSLN binding protein, according to any one of the above embodiments, is a single domain antibody comprising the sequence of SEQ ID NO: 627. In some embodiments, the MSLN binding protein, according to any one of the above embodiments, is a single domain antibody comprising the sequence of SEQ ID NO: 628. In some embodiments, the MSLN binding protein, according to any one of the above embodiments, is a single domain antibody comprising the sequence of SEQ ID NO: 629. In some embodiments, the MSLN binding protein, according to any one of the above embodiments, is a single domain antibody comprising the sequence of SEQ ID NO: 630. In some embodiments, the MSLN binding protein, according to any one of the above embodiments, is a single domain antibody comprising the sequence of SEQ ID NO: 631. In some embodiments, the MSLN binding protein, according to any one of the above embodiments, is a single domain antibody comprising the sequence of SEQ ID NO: 632. In some embodiments, the MSLN binding protein, according to any one of the above embodiments, is a single domain antibody comprising the sequence of SEQ ID NO: 633. In some embodiments, the MSLN binding protein, according to any one of the above embodiments, is a single domain antibody comprising the sequence of SEQ ID NO: 634. In some embodiments, the MSLN binding protein, according to any one of the above embodiments, is a single domain antibody comprising the sequence of SEQ ID NO: 635.

In some embodiments, the MSLN binding protein, according to any one of the above embodiments, is a humanized single domain antibody comprising the sequence of SEQ ID NO: 636. In some embodiments, the MSLN binding protein, according to any one of the above embodiments, is a humanized single domain antibody comprising the sequence of SEQ ID NO: 637. In some embodiments, the MSLN binding protein, according to any one of the above embodiments, is a humanized single domain antibody comprising the sequence of SEQ ID NO: 638. In some embodiments, the MSLN binding protein, according to any one of the above embodiments, is a humanized single domain antibody comprising the sequence of SEQ ID NO: 639. In some embodiments, the MSLN binding protein, according to any one of the above embodiments, is a humanized single domain antibody comprising the sequence of SEQ ID NO: 640. In some embodiments, the MSLN binding protein, according to any one of the above embodiments, is a humanized single domain antibody comprising the sequence of SEQ ID NO: 641. In some embodiments, the MSLN binding protein, according to any one of the above embodiments, is a humanized single domain antibody comprising the sequence of SEQ ID NO: 642. In some embodiments, the MSLN binding protein, according to any one of the above embodiments, is a humanized single domain antibody comprising the sequence of SEQ ID NO: 643. In some embodiments, the MSLN binding protein, according to any one of the above embodiments, is a humanized single domain antibody comprising the sequence of SEQ ID NO: 644. In some embodiments, the MSLN binding protein, according to any one of the above embodiments, is a humanized single domain antibody comprising the sequence of SEQ ID NO: 645. In some embodiments, the MSLN binding protein, according to any one of the above embodiments, is a humanized single domain antibody comprising the sequence of SEQ ID NO: 646. In some embodiments, the MSLN binding protein, according to any one of the above embodiments, is a humanized single domain antibody comprising the sequence of SEQ ID NO: 647. In some embodiments, the MSLN binding protein, according to any one of the above embodiments, is a humanized single domain antibody comprising the sequence of SEQ ID NO: 648. In some embodiments, the MSLN binding protein, according to any one of the above embodiments, is a humanized single domain antibody comprising the sequence of SEQ ID NO: 649. In some embodiments, the MSLN binding protein, according to any one of the above embodiments, is a humanized single domain antibody comprising the sequence of SEQ ID NO: 650.

In some embodiments, the MSLN binding domain is cross-reactive with human and cynomolgus mesothelin. In some embodiments, the MSLN binding domain is specific for human mesothelin. In certain embodiments, the MSLN binding domains disclosed herein bind to human mesothelin with a human Kd (hKd). In certain embodiments, the MSLN binding domains disclosed herein bind to cynomolgus mesothelin with a cyno Kd (cKd). In certain embodiments, the MSLN binding domains disclosed herein bind to both cynomolgus mesothelin and a human mesothelin, with a cyno Kd (cKd) and a human Kd (hKd), respectively. In some embodiments, the MSLN binding protein binds to human and cynomolgus mesothelin with comparable binding affinities (i.e., hKd and cKd values do not differ by more than ±10%). In some embodiments, the hKd and the cKd range from about 0.1 nM to about 500 nM. In some embodiments, the hKd and the cKd range from about 0.1 nM to about 450 nM. In some embodiments, the hKd and the cKd range from about 0.1 nM to about 400 nM. In some embodiments, the hKd and the cKd range from about 0.1 nM to about 350 nM. In some embodiments, the hKd and the cKd range from about 0.1 nM to about 300 nM. In some embodiments, the hKd and the cKd range from about 0.1 nM to about 250 nM. In some embodiments, the hKd and the cKd range from about 0.1 nM to about 200 nM. In some embodiments, the hKd and the cKd range from about 0.1 nM to about 150 nM. In some embodiments, the hKd and the cKd range from about 0.1 nM to about 100 nM. In some embodiments, the hKd and the cKd range from about 0.1 nM to about 90 nM. In some embodiments, the hKd and the cKd range from about 0.2 nM to about 80 nM. In some embodiments, the hKd and the cKd range from about 0.3 nM to about 70 nM. In some embodiments, the hKd and the cKd range from about 0.4 nM to about 50 nM. In some embodiments, the hKd and the cKd range from about 0.5 nM to about 30 nM. In some embodiments, the hKd and the cKd range from about 0.6 nM to about 10 nM. In some embodiments, the hKd and the cKd range from about 0.7 nM to about 8 nM. In some embodiments, the hKd and the cKd range from about 0.8 nM to about 6 nM. In some embodiments, the hKd and the cKd range from about 0.9 nM to about 4 nM. In some embodiments, the hKd and the cKd range from about 1 nM to about 2 nM.

In some embodiments, any of the foregoing MSLN binding domains (e.g., anti-MSLN single domain antibodies of SEQ ID NOS: 607-646) are affinity peptide tagged for ease of purification. In some embodiments, the affinity peptide tag is six consecutive histidine residues, also referred to as 6X-his (SEQ ID NO: 3503).

In certain embodiments, the MSLN binding domains of the present disclosure preferentially bind membrane bound mesothelin over soluble mesothelin. Membrane bound mesothelin refers to the presence of mesothelin in or on the cell membrane surface of a cell that expresses mesothelin. Soluble mesothelin refers to mesothelin that is no longer on in or on the cell membrane surface of a cell that expresses or expressed mesothelin. In certain instances, the soluble mesothelin is present in the blood and/or lymphatic circulation in a subject. In one embodiment, the MSLN binding domains bind membrane-bound mesothelin at least 5 fold, 10 fold, 15 fold, 20 fold, 25 fold, 30 fold, 40 fold, 50 fold, 100 fold, 500 fold, or 1000 fold greater than soluble mesothelin. In one embodiment, the MSLN targeting immune cell engaging proteins of the present disclosure preferentially bind membrane-bound mesothelin 30 fold greater than soluble mesothelin. Determining the preferential binding of an antigen binding protein to membrane bound MSLN over soluble MSLN can be readily determined using assays well known in the art.

### B Cell Maturation Antigen (BCMA) Binding Proteins

B cell maturation antigen (BCMA, TNFRSF17, CD269) is a transmembrane protein belonging to the tumor necrosis family receptor (TNFR) super family that is primarily expressed on terminally differentiated B cells. BCMA expression is restricted to the B cell lineage and mainly present on plasma cells and plasmablasts and to some extent on memory B cells, but virtually absent on peripheral and naive B cells. BCMA is also expressed on multiple myeloma (MM) cells, on leukemia cells and lymphoma cells.

BCMA was identified through molecular analysis of a t(4;16)(q26;p13) translocation found in a human intestinal T cell lymphoma and an in-frame sequence was mapped to the 16p13.1 chromosome band.

Human BCMA cDNA has an open reading frame of 552 bp that encodes a 184 amino acid polypeptide. The BCMA gene is organized into three exons that are separated by two introns, each flanked by GT donor and AG acceptor consensus splicing sites, and codes for a transcript of 1.2 kb. The structure of BCMA protein includes an integral transmembrane protein based on a central 24 amino acid hydrophobic region in an alpha-helix structure.

The murine BCMA gene is located on chromosome 16 syntenic to the human 1⁶p13 region, and also includes three exons that are separated by two introns. The gene encodes a 185 amino acid protein. Murine BCMA mRNA is expressed as a 404 bp transcript at the highest levels in plasmacytoma cells (J558) and at modest levels in the A20 B cell lymphoma line. Murine BCMA mRNA transcripts have also been detected at low levels in T cell lymphoma (EL4, BW5147) and dendritic cell (CB1D6, D2SC1) lines in contrast to human cell lines of T cell and dendritic cell origin. The murine BCMA cDNA sequence has 69.3% nucleotide identity with the human BCMA cDNA sequence and slightly higher identity (73.7%) when comparing the coding regions between these two cDNA sequences. Mouse BCMA protein is 62% identical to human BCMA protein and, like human BCMA, contains a single hydrophobic region, which may be an internal transmembrane segment. The N-terminal 40 amino acid domain of both murine and human BCMA protein have six conserved cysteine residues, consistent with the formation of a cysteine repeat motif found in the extracellular domain of TNFRs. Similar to members of the TNFR superfamily, BCMA protein contains a conserved aromatic residue four to six residues C-terminal from the first cysteine.

BCMA is not expressed at the cell surface, but rather, is located on the Golgi apparatus. The amount of BCMA expression is proportional to the stage of cellular differentiation (highest in plasma cells).

BCMA is involved in B cell development and homeostasis due to its interaction with its ligands BAFF (B cell activating factor, also designated as TALL-1 or TNFSF13B) and APRIL (A proliferation inducing ligand). BCMA regulates different aspects of humoral immunity, B cell development and homeostasis along with its family members TACI (transmembrane activator and cyclophylin ligand interactor) and BAFF-R (B cell activation factor receptor, also known as tumor necrosis factor receptor superfamily member 13C). Expression of BCMA appears rather late in B cell differentiation and contributes to the long-term survival of plasmablasts and plasma cells in the bone marrow. BCMA also supports growth and survival of multiple myeloma (MM) cells. BCMA is mostly known for its functional activity in mediating the survival of plasma cells that maintain long-term humoral immunity.

There is a need for having treatment options for solid tumor diseases related to the overexpression of BCMA, such as cancer multiple myeloma, leukemias and lymphomas. The present disclosure provides, in certain embodiments, single domain proteins which specifically bind to BCMA on the surface of tumor target cells.

The design of the BCMA targeting immune cell engaging proteins described herein allows the binding domain to BCMA to be flexible in that the binding domain to BCMA can be any type of binding domain, including but not limited to, domains from a monoclonal antibody, a polyclonal antibody, a recombinant antibody, a human antibody, a humanized antibody. In some embodiments, the binding domain to BCMA is a single chain variable fragments (scFv), single-domain antibody such as a heavy chain variable domain (VH), a light chain variable domain (VL) and a variable domain (VHH) of camelid derived single domain antibody. In other embodiments, the binding domain to BCMA is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to BCMA is a ligand or peptide that binds to or associates with BCMA. In yet further embodiments, the binding domain to BCMA is a knottin. In yet further embodiments, the binding domain to BCMA is a small molecular entity.

In some embodiments, the BCMA binding domain binds to a protein comprising the sequence of SEQ ID NO: 3201, 3202 or 3203. In some embodiments, the BCMA binding domain binds to a protein comprising a truncated sequence compared to SEQ ID NO: 3201, 3202 or 3203.

In some embodiments, the BCMA binding domain is an anti-BCMA antibody or an antibody variant. As used herein, the term “antibody variant” refers to variants and derivatives of an antibody described herein. In certain embodiments, amino acid sequence variants of the anti-BCMA antibodies described herein are contemplated. For example, in certain embodiments amino acid sequence variants of anti-BCMA antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant anti-BCMA antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the BCMA binding domain of the BCMA targeting immune cell engaging protein is a single domain antibody such as a heavy chain variable domain (VH), a variable domain (VHH) of a llama derived sdAb, a peptide, a ligand or a small molecule entity specific for BCMA. In some embodiments, the BCMA binding domain of the BCMA targeting immune cell engaging protein described herein is any domain that binds to BCMA including but not limited to domains from a monoclonal antibody, a polyclonal antibody, a recombinant antibody, a human antibody, a humanized antibody. In certain embodiments, the BCMA binding domain is a single-domain antibody. In other embodiments, the BCMA binding domain is a peptide. In further embodiments, the BCMA binding domain is a small molecule.

In one embodiment, a single domain antibody corresponds to the VHH domains of naturally occurring heavy chain antibodies directed against BCMA. As further described herein, such VHH sequences can generally be generated or obtained by suitably immunizing a species of Llama with BCMA, (i.e., so as to raise an immune response and/or heavy chain antibodies directed against BCMA), by obtaining a suitable biological sample from said Llama (such as a blood sample, serum sample or sample of B-cells), and by generating VHH sequences directed against BCMA, starting from said sample, using any suitable technique known in the field.

In another embodiment, such naturally occurring VHH domains against BCMA, are obtained from naïve libraries of Camelid VHH sequences, for example by screening such a library using BCMA, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against BCMA, involves suitably immunizing a transgenic mammal that is capable of expressing heavy chain antibodies (i.e., so as to raise an immune response and/or heavy chain antibodies directed against BCMA), obtaining a suitable biological sample from said transgenic mammal (such as a blood sample, serum sample or sample of B-cells), and then generating VHH sequences directed against BCMA, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-BCMA single domain antibody of the BCMA targeting immune cell engaging protein comprises a single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining the anti-BCMA single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-BCMA single domain antibody of the disclosure or a nucleotide sequence or nucleic acid encoding the same.

In some embodiments, the BCMA binding domain is an anti-BCMA specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the BCMA binding domain comprises any domain that binds to BCMA including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the BCMA binding domain is a single domain antibody. In some embodiments, the anti-BCMA single domain antibody comprises heavy chain variable complementarity determining regions (CDR), CDR1, CDR2, and CDR3.

In some embodiments, the BCMA binding protein of the present disclosure is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The r1 residues of the BCMA binding protein of the present disclosure comprise, for example, amino acid residues 26, 27, 28, 29, 30, 31, 32, 33 and 34; the r2 residues of the BCMA binding protein of the present disclosure comprise, for example, amino acid residues, for example, 50, 51, 52, 53, 54, 55, 56, 57, 58, 59, 60, 61, 62 and 63; and the r3 residues of the BCMA binding protein of the present disclosure comprise, for example, amino acid residues, for example, 97, 98, 99, 100, 101, 102, 103, 104, 105, 106, 107 and 108. In some embodiments, the BCMA binding protein comprises an amino acid sequence selected from the group consisting of SEQ ID NOS: 346-460.

In some embodiments, an exemplary CDR1 comprises the amino acid sequence as set forth in SEQ ID NO: 1-115. In some embodiments, another exemplary CDR2 comprises the amino acid sequence as set forth in SEQ ID NO: 116-230. In some embodiments, another exemplary CDR3 comprises the amino acid sequence as set forth in SEQ ID NO: 231-345.

In various embodiments, the BCMA binding protein of the present disclosure has a CDR1 that has an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 1-115.

In various embodiments, the BCMA binding protein of the present disclosure has a CDR2 that has an amino acid sequence that is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 116-230.

In various embodiments, a complementarity determining region of the BCMA binding protein of the present disclosure has a CDR3 that has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 231-345.

In various embodiments, a BCMA binding protein of the present disclosure has an amino acid sequence that is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 346-460.

In some embodiments, the BCMA binding protein is a single domain antibody comprising the sequence of SEQ ID NO: 346. In some embodiments, the BCMA binding protein is a single domain antibody comprising the sequence of SEQ ID NO: 347. In some embodiments, the BCMA binding protein is a single domain antibody comprising the sequence of SEQ ID NO: 348. In some embodiments, the BCMA binding protein is a single domain antibody comprising the sequence of SEQ ID NO: 349. In some embodiments, the BCMA binding protein is a single domain antibody comprising the sequence of SEQ ID NO: 350. In some embodiments, the BCMA binding protein is a single domain antibody comprising the sequence of SEQ ID NO: 351. In some embodiments, the BCMA binding protein is a single domain antibody comprising the sequence of SEQ ID NO: 352. In some embodiments, the BCMA binding protein is a single domain antibody comprising the sequence of SEQ ID NO: 353. In some embodiments, the BCMA binding protein is a single domain antibody comprising the sequence of SEQ ID NO: 354. In some embodiments, the BCMA binding protein is a single domain antibody comprising the sequence of SEQ ID NO: 355. In some embodiments, the BCMA binding protein is a single domain antibody comprising the sequence of SEQ ID NO: 356. In some embodiments, the BCMA binding protein is a single domain antibody comprising the sequence of SEQ ID NO: 357. In some embodiments, the BCMA binding protein is a single domain antibody comprising the sequence of SEQ ID NO: 358. In some embodiments, the BCMA binding protein is a single domain antibody comprising the sequence of SEQ ID NO: 359.

In some embodiments, the BCMA binding protein is a single domain antibody comprising the sequence of SEQ ID NO: 360. In some embodiments, the BCMA binding protein is a single domain antibody comprising the sequence of SEQ ID NO: 361. In some embodiments, the BCMA binding protein is a single domain antibody comprising the sequence of SEQ ID NO: 362. In some embodiments, the BCMA binding protein is a single domain antibody comprising the sequence of SEQ ID NO: 363. In some embodiments, the BCMA binding protein is a single domain antibody comprising the sequence of SEQ ID NO: 364. In some embodiments, the BCMA binding protein is a single domain antibody comprising the sequence of SEQ ID NO: 365. In some embodiments, the BCMA binding protein is a single domain antibody comprising the sequence of SEQ ID NO: 366. In some embodiments, the BCMA binding protein is a single domain antibody comprising the sequence of SEQ ID NO: 367. In some embodiments, the BCMA binding protein is a single domain antibody comprising the sequence of SEQ ID NO: 368. In some embodiments, the BCMA binding protein is a single domain antibody comprising the sequence of SEQ ID NO: 369.

In some embodiments, the BCMA binding protein is a single domain antibody comprising the sequence of SEQ ID NO: 370. In some embodiments, the BCMA binding protein is a single domain antibody comprising the sequence of SEQ ID NO: 371. In some embodiments, the BCMA binding protein is a single domain antibody comprising the sequence of SEQ ID NO: 372. In some embodiments, the BCMA binding protein is a single domain antibody comprising the sequence of SEQ ID NO: 373. In some embodiments, the BCMA binding protein is a single domain antibody comprising the sequence of SEQ ID NO: 374. In some embodiments, the BCMA binding protein is a single domain antibody comprising the sequence of SEQ ID NO: 375. In some embodiments, the BCMA binding protein is a single domain antibody comprising the sequence of SEQ ID NO: 376. In some embodiments, the BCMA binding protein is a single domain antibody comprising the sequence of SEQ ID NO: 377. In some embodiments, the BCMA binding protein is a single domain antibody comprising the sequence of SEQ ID NO: 378. In some embodiments, the BCMA binding protein is a single domain antibody comprising the sequence of SEQ ID NO: 379.

In some embodiments, the BCMA binding protein is a single domain antibody comprising the sequence of SEQ ID NO: 380. In some embodiments, the BCMA binding protein is a single domain antibody comprising the sequence of SEQ ID NO: 381. In some embodiments, the BCMA binding protein is a single domain antibody comprising the sequence of SEQ ID NO: 382. In some embodiments, the BCMA binding protein is a single domain antibody comprising the sequence of SEQ ID NO: 383. In some embodiments, the BCMA binding protein is a single domain antibody comprising the sequence of SEQ ID NO: 384. In some embodiments, the BCMA binding protein is a single domain antibody comprising the sequence of SEQ ID NO: 385. In some embodiments, the BCMA binding protein is a single domain antibody comprising the sequence of SEQ ID NO: 386. In some embodiments, the BCMA binding protein is a single domain antibody comprising the sequence of SEQ ID NO: 387. In some embodiments, the BCMA binding protein is a single domain antibody comprising the sequence of SEQ ID NO: 388. In some embodiments, the BCMA binding protein is a single domain antibody comprising the sequence of SEQ ID NO: 389.

In some embodiments, the BCMA binding protein is a single domain antibody comprising the sequence of SEQ ID NO: 390. In some embodiments, the BCMA binding protein is a single domain antibody comprising the sequence of SEQ ID NO: 391. In some embodiments, the BCMA binding protein is a single domain antibody comprising the sequence of SEQ ID NO: 392. In some embodiments, the BCMA binding protein is a single domain antibody comprising the sequence of SEQ ID NO: 393. In some embodiments, the BCMA binding protein is a single domain antibody comprising the sequence of SEQ ID NO: 394. In some embodiments, the BCMA binding protein is a single domain antibody comprising the sequence of SEQ ID NO: 395. In some embodiments, the BCMA binding protein is a single domain antibody comprising the sequence of SEQ ID NO: 396. In some embodiments, the BCMA binding protein is a single domain antibody comprising the sequence of SEQ ID NO: 397. In some embodiments, the BCMA binding protein is a single domain antibody comprising the sequence of SEQ ID NO: 398. In some embodiments, the BCMA binding protein is a single domain antibody comprising the sequence of SEQ ID NO: 399.

In some embodiments, the BCMA binding protein is a single domain antibody comprising the sequence of SEQ ID NO: 400. In some embodiments, the BCMA binding protein is a single domain antibody comprising the sequence of SEQ ID NO: 401. In some embodiments, the BCMA binding protein is a single domain antibody comprising the sequence of SEQ ID NO: 402. In some embodiments, the BCMA binding protein is a single domain antibody comprising the sequence of SEQ ID NO: 403. In some embodiments, the BCMA binding protein is a single domain antibody comprising the sequence of SEQ ID NO: 404. In some embodiments, the BCMA binding protein is a single domain antibody comprising the sequence of SEQ ID NO: 405. In some embodiments, the BCMA binding protein is a single domain antibody comprising the sequence of SEQ ID NO: 406. In some embodiments, the BCMA binding protein is a single domain antibody comprising the sequence of SEQ ID NO: 407. In some embodiments, the BCMA binding protein is a single domain antibody comprising the sequence of SEQ ID NO: 408. In some embodiments, the BCMA binding protein is a single domain antibody comprising the sequence of SEQ ID NO: 409.

In some embodiments, the BCMA binding protein is a single domain antibody comprising the sequence of SEQ ID NO: 410. In some embodiments, the BCMA binding protein is a single domain antibody comprising the sequence of SEQ ID NO: 411. In some embodiments, the BCMA binding protein is a single domain antibody comprising the sequence of SEQ ID NO: 412. In some embodiments, the BCMA binding protein is a single domain antibody comprising the sequence of SEQ ID NO: 413. In some embodiments, the BCMA binding protein is a single domain antibody comprising the sequence of SEQ ID NO: 414. In some embodiments, the BCMA binding protein is a single domain antibody comprising the sequence of SEQ ID NO: 415. In some embodiments, the BCMA binding protein is a single domain antibody comprising the sequence of SEQ ID NO: 416. In some embodiments, the BCMA binding protein is a single domain antibody comprising the sequence of SEQ ID NO: 417. In some embodiments, the BCMA binding protein is a single domain antibody comprising the sequence of SEQ ID NO: 418. In some embodiments, the BCMA binding protein is a single domain antibody comprising the sequence of SEQ ID NO: 419.

In some embodiments, the BCMA binding protein is a single domain antibody comprising the sequence of SEQ ID NO: 420. In some embodiments, the BCMA binding protein is a single domain antibody comprising the sequence of SEQ ID NO: 421. In some embodiments, the BCMA binding protein is a single domain antibody comprising the sequence of SEQ ID NO: 422. In some embodiments, the BCMA binding protein is a single domain antibody comprising the sequence of SEQ ID NO: 423. In some embodiments, the BCMA binding protein is a single domain antibody comprising the sequence of SEQ ID NO: 424. In some embodiments, the BCMA binding protein is a single domain antibody comprising the sequence of SEQ ID NO: 425. In some embodiments, the BCMA binding protein is a single domain antibody comprising the sequence of SEQ ID NO: 426. In some embodiments, the BCMA binding protein is a single domain antibody comprising the sequence of SEQ ID NO: 427. In some embodiments, the BCMA binding protein is a single domain antibody comprising the sequence of SEQ ID NO: 428. In some embodiments, the BCMA binding protein is a single domain antibody comprising the sequence of SEQ ID NO: 429.

In some embodiments, the BCMA binding protein is a single domain antibody comprising the sequence of SEQ ID NO: 430. In some embodiments, the BCMA binding protein is a single domain antibody comprising the sequence of SEQ ID NO: 431. In some embodiments, the BCMA binding protein is a single domain antibody comprising the sequence of SEQ ID NO: 432. In some embodiments, the BCMA binding protein is a single domain antibody comprising the sequence of SEQ ID NO: 433. In some embodiments, the BCMA binding protein is a single domain antibody comprising the sequence of SEQ ID NO: 434. In some embodiments, the BCMA binding protein is a single domain antibody comprising the sequence of SEQ ID NO: 435. In some embodiments, the BCMA binding protein is a single domain antibody comprising the sequence of SEQ ID NO: 436. In some embodiments, the BCMA binding protein is a single domain antibody comprising the sequence of SEQ ID NO: 437. In some embodiments, the BCMA binding protein is a single domain antibody comprising the sequence of SEQ ID NO: 438. In some embodiments, the BCMA binding protein is a single domain antibody comprising the sequence of SEQ ID NO: 439.

In some embodiments, the BCMA binding protein is a single domain antibody comprising the sequence of SEQ ID NO: 440. In some embodiments, the BCMA binding protein is a single domain antibody comprising the sequence of SEQ ID NO: 441. In some embodiments, the BCMA binding protein is a single domain antibody comprising the sequence of SEQ ID NO: 442. In some embodiments, the BCMA binding protein is a single domain antibody comprising the sequence of SEQ ID NO: 443. In some embodiments, the BCMA binding protein is a single domain antibody comprising the sequence of SEQ ID NO: 444. In some embodiments, the BCMA binding protein is a single domain antibody comprising the sequence of SEQ ID NO: 445. In some embodiments, the BCMA binding protein is a single domain antibody comprising the sequence of SEQ ID NO: 446. In some embodiments, the BCMA binding protein is a single domain antibody comprising the sequence of SEQ ID NO: 447. In some embodiments, the BCMA binding protein is a single domain antibody comprising the sequence of SEQ ID NO: 448. In some embodiments, the BCMA binding protein is a single domain antibody comprising the sequence of SEQ ID NO: 449.

In some embodiments, the BCMA binding protein is a single domain antibody comprising the sequence of SEQ ID NO: 450. In some embodiments, the BCMA binding protein is a single domain antibody comprising the sequence of SEQ ID NO: 451. In some embodiments, the BCMA binding protein is a single domain antibody comprising the sequence of SEQ ID NO: 452. In some embodiments, the BCMA binding protein is a single domain antibody comprising the sequence of SEQ ID NO: 453. In some embodiments, the BCMA binding protein is a single domain antibody comprising the sequence of SEQ ID NO: 454. In some embodiments, the BCMA binding protein is a single domain antibody comprising the sequence of SEQ ID NO: 455. In some embodiments, the BCMA binding protein is a single domain antibody comprising the sequence of SEQ ID NO: 456. In some embodiments, the BCMA binding protein is a single domain antibody comprising the sequence of SEQ ID NO: 457. In some embodiments, the BCMA binding protein is a single domain antibody comprising the sequence of SEQ ID NO: 458. In some embodiments, the BCMA binding protein is a single domain antibody comprising the sequence of SEQ ID NO: 459. In some embodiments, the BCMA binding protein is a single domain antibody comprising the sequence of SEQ ID NO: 460.

A BCMA binding protein described herein can bind to human BCMA with a hKd ranges from about 0.1 nM to about 500 nM. In some embodiments, the hKd ranges from about 0.1 nM to about 450 nM. In some embodiments, the hKd ranges from about 0.1 nM to about 400 nM. In some embodiments, the hKd ranges from about 0.1 nM to about 350 nM. In some embodiments, the hKd ranges from about 0.1 nM to about 300 nM. In some embodiments, the hKd ranges from about 0.1 nM to about 250 nM. In some embodiments, the hKd ranges from about 0.1 nM to about 200 nM. In some embodiments, the hKd ranges from about 0.1 nM to about 150 nM. In some embodiments, the hKd ranges from about 0.1 nM to about 100 nM. In some embodiments, the hKd ranges from about 0.1 nM to about 90 nM. In some embodiments, the hKd ranges from about 0.2 nM to about 80 nM. In some embodiments, the hKd ranges from about 0.3 nM to about 70 nM. In some embodiments, the hKd ranges from about 0.4 nM to about 50 nM. In some embodiments, the hKd ranges from about 0.5 nM to about 30 nM. In some embodiments, the hKd ranges from about 0.6 nM to about 10 nM. In some embodiments, the hKd ranges from about 0.7 nM to about 8 nM. In some embodiments, the hKd ranges from about 0.8 nM to about 6 nM. In some embodiments, the hKd ranges from about 0.9 nM to about 4 nM. In some embodiments, the hKd ranges from about 1 nM to about 2 nM.

In some embodiments, any of the foregoing BCMA binding domains are affinity peptide tagged for ease of purification. In some embodiments, the affinity peptide tag is six consecutive histidine residues, also referred to as a His tag or 6X-his (His-His-His-His-His-His; SEQ ID NO: 3503).

In certain embodiments, the BCMA binding domains of the present disclosure preferentially bind membrane bound BCMA over soluble BCMA. Membrane bound BCMA refers to the presence of BCMA in or on the cell membrane surface of a cell that expresses BCMA. Soluble BCMA refers to BCMA that is no longer on in or on the cell membrane surface of a cell that expresses or expressed BCMA. In certain instances, the soluble BCMA is present in the blood and/or lymphatic circulation in a subject. In one embodiment, the BCMA binding domains bind membrane-bound BCMA at least 5 fold, 10 fold, 15 fold, 20 fold, 25 fold, 30 fold, 40 fold, 50 fold, 100 fold, 500 fold, or 1000 fold greater than soluble BCMA. In one embodiment, the BCMA targeting immune cell engaging proteins of the present disclosure preferentially bind membrane-bound BCMA 30 fold greater than soluble BCMA. Determining the preferential binding of an antigen binding protein to membrane bound BCMA over soluble BCMA can be readily determined using assays well known in the art.

### Delta-Like Ligand 3 (DLL3) Binding Proteins

DLL3 (also known as Delta-like Ligand 3 or SCDO1) is a member of the Delta-like family of Notch DSL ligands. Representative DLL3 protein orthologs include, but are not limited to, human (Accession Nos. NP_058637 and NP_982353), chimpanzee (Accession No. XP_003316395), mouse (Accession No. NP_031892), and rat (Accession No. NP_446118). In humans, the DLL3 gene consists of 8 exons spanning 9.5 kbp located on chromosome 19q13. Alternate splicing within the last exon gives rise to two processed transcripts, one of 2389 bases (Accession No. NM_016941) and one of 2052 bases (Accession No. NM_203486). The former transcript encodes a 618 amino acid protein (Accession No. NP_058637), whereas the latter encodes a 587 amino acid protein (Accession No. NP_982353). These two protein isoforms of DLL3 share overall 100% identity across their extracellular domains and their transmembrane domains, differing only in that the longer isoform contains an extended cytoplasmic tail containing 32 additional residues at the carboxy terminus of the protein. The extracellular region of the DLL3 protein, comprises six EGF-like domains, the single DSL domain and the N-terminal domain. Generally, the EGF domains are recognized as occurring at about amino acid residues 216-249 (domain 1), 274-310 (domain 2), 312-351 (domain 3), 353-389 (domain 4), 391-427 (domain 5) and 429-465 (domain 6), with the DSL domain at about amino acid residues 176-215 and the N-terminal domain at about amino acid residues 27-175 of hDLL3. Each of the EGF-like domains, the DSL domain and the N-terminal domain comprise part of the DLL3 protein as defined by a distinct amino acid sequence. The EGF-like domains are termed, in some embodiments, as EGF1 to EGF6 with EGF1 being closest to the N-terminal portion of the protein. In general, DSL ligands are composed of a series of structural domains: a unique N-terminal domain, followed by a conserved DSL domain, multiple tandem epidermal growth factor (EGF)-like repeats, a transmembrane domain, and a cytoplasmic domain not highly conserved across ligands but one which contains multiple lysine residues that are potential sites for ubiquitination by unique E3 ubiquitin ligases. The DSL domain is a degenerate EGF-domain that is necessary but not sufficient for interactions with Notch receptors. Additionally, the first two EGF-like repeats of most DSL ligands contain a smaller protein sequence motif known as a DOS domain that co-operatively interacts with the DSL domain when activating Notch signaling.

In some embodiments, the disclosed DLL3 immune cell engaging proteins of this disclosure are generated, fabricated, engineered or selected so as to react with a selected domain, motif or epitope within a DLL3 protein. In some embodiments, the DLL3 targeting immune cell engaging protein binds to the DSL domain and, in some embodiments, binds to an epitope comprising G203, R205, P206 within the DSL domain.

The DLL3 binding domain of the DLL3 targeting immune cell engaging proteins of the present disclosure are, in some embodiments, engineered fabricated and/or selected to react with both isoform(s) of DLL3 or a single isoform of the protein or, conversely, comprise a pan-DLL binding domain that reacts or associates with at least one additional DLL family member in addition to DLL3. In some embodiments, the DLL3 binding domain, such as DLL3 binding domain are engineered, fabricated, and/or selected so that they react with domains (or epitopes therein) that are exhibited by DLL3 only or with domains that are at least somewhat conserved across multiple or all DLL family members.

In some embodiments the DLL3 binding domain associates or binds to a specific epitope, portion, motif or domain of DLL3. Both DLL3 isoforms incorporate an identical extracellular region comprising at least an N-terminal domain, a DSL (Delta/Serrate/lag-2) domain and six EGF-like domains (i.e., EGF1-EGF6). Accordingly, in certain embodiments the DLL3 binding domain binds or associate with the N-terminal domain of DLL3 (amino acids 27-175 in the mature protein) while in other embodiments the DLL3 binding domain associates with the DSL domain (amino acids 176-215) or epitope therein. In other aspects of the present disclosure the DLL3 binding domain associates or bind to a specific epitope located in a particular EGF-like domain of DLL3. In some embodiments, the DLL3 binding domain associates or binds to an epitope located in EGF1 (amino acids 216-249), EGF2 (amino acids 274-310), EGF3 (amino acids 312-351), EGF4 (amino acids 353-389), EGF5 (amino acids 391.427) or EGF6 (amino acids 429-465). In some embodiments, each of the aforementioned domains comprises more than one epitope and/or more than one bin. In some embodiments the DLL3 binding domain binds, reacts or associates with the DSL domain or an epitope therein. In other embodiments the DLL3 binding domain binds, reacts or associates with a particular EGF-like domain or an epitope therein. In some embodiments the DLL3 binding domain binds, reacts or associates with the N-terminal domain or an epitope therein.

In some embodiments, the DLL3 binding proteins of this disclosure, such as the DLL3 binding domain of the immune cell engaging proteins of this disclosure binds to the full length DLL3 protein or to a fragment thereof, such as epitope containing fragments within the full length DLL3 protein, as described above. In some cases, the epitope containing fragment comprises antigenic or immunogenic fragments and derivatives thereof of the DLL3 protein. Epitope containing fragments, including antigenic or immunogenic fragments, are, in some embodiments, 12 amino acids or more, 20 amino acids or more, 50 or 100 amino acids or more. The DLL3 fragments, in some embodiments, comprises 95% or more of the length of the full protein, 90% or more, 75% or 50% or 25% or 10% or more of the length of the full protein. In some embodiments, the epitope-containing fragments of DLL3 including antigenic or immunogenic fragments are capable of eliciting a relevant immune response in a patient. Derivatives of DLL3 include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the DLL3 sequence provided in SEQ ID NO: 3216 (UniProtKB Accession Q9NYJ7). In some embodiments, substitutions comprise conservative substitutions. Derivatives and variants of DLL3, in some examples, have essentially the same biological function as the DLL3 protein from which they are derived. For instance, derivatives and variants of DLL3 are, in some cases, comparably antigenic or immunogenic to the protein from which they are derived, have either the ligand-binding activity, or the active receptor-complex forming ability, or preferably both, of the protein from which they are derived, and have the same tissue distribution as DLL3.

The design of the DLL3 targeting immune cell engaging proteins described herein allows the binding domain to DLL3 to be flexible in that the binding domain to DLL3 can be any type of binding domain, including but not limited to, domains from a monoclonal antibody, a polyclonal antibody, a recombinant antibody, a human antibody, a humanized antibody. In some embodiments, the binding domain to DLL3 is a single chain variable fragments (scFv), a single-domain antibody such as a heavy chain variable domain (VH), a light chain variable domain (VL) and a variable domain (VHH) of camelid derived single domain antibody. In other embodiments, the binding domain to DLL3 is a non-Ig binding domain, i.e., an antibody mimetic, such as anticalins, affilins, affibody molecules, affimers, affitins, alphabodies, avimers, DARPins, fynomers, kunitz domain peptides, and monobodies. In further embodiments, the binding domain to DLL3 is a ligand or peptide that binds to or associates with DLL3. In yet further embodiments, the binding domain to DLL3 is a knottin. In yet further embodiments, the binding domain to DLL3 is a small molecular entity.

In some embodiments, the DLL3 binding domain binds to a protein comprising the sequence of SEQ ID NO: 3216 (UniProtKB Accession Q9NYJ7). In some embodiments, the DLL3 binding domain binds to a protein comprising a truncated sequence compared to SEQ ID NO: 3216 (UniProtKB Accession Q9NYJ7). In some embodiments, the DLL3 binding domain binds to a protein comprising the sequence of SEQ ID NO: 3509 or SEQ ID NO: 3217 (which is the mature extracellular domain of a DLL3 protein). In some embodiments, the DLL3 binding domain binds to a protein comprising amino acids 47-492 of SEQ ID NO: 3509. In some embodiments, the DLL3 binding domain recognizes an epitope within amino acids 47-4492 of SEQ ID NO: 3509.

In some embodiments, the DLL3 binding domain is an anti-DLL3 antibody or an antibody variant. As used herein, the term “antibody variant” refers to variants and derivatives of an antibody described herein. In certain embodiments, amino acid sequence variants of the anti-DLL3 antibodies described herein are contemplated. For example, in certain embodiments amino acid sequence variants of anti-DLL3 antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant anti-DLL3 antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the DLL3 binding domain of the DLL3 targeting immune cell engaging protein is a single domain antibody such as a heavy chain variable domain (VH), a variable domain (VHH) of a llama derived sdAb, a peptide, a ligand or a small molecule entity specific for DLL3. In some embodiments, the DLL3 binding domain of the DLL3 targeting immune cell engaging protein described herein is any domain that binds to DLL3 including but not limited to domains from a monoclonal antibody, a polyclonal antibody, a recombinant antibody, a human antibody, a humanized antibody. In certain embodiments, the DLL3 binding domain is a single-domain antibody. In other embodiments, the DLL3 binding domain is a peptide. In further embodiments, the DLL3 binding domain is a small molecule.

In one embodiment, a single domain antibody corresponds to the VHH domains of naturally occurring heavy chain antibodies directed against DLL3. As further described herein, such VHH sequences can generally be generated or obtained by suitably immunizing a species of Llama with DLL3, (i.e., so as to raise an immune response and/or heavy chain antibodies directed against DLL3), by obtaining a suitable biological sample from said Llama (such as a blood sample, serum sample or sample of B-cells), and by generating VHH sequences directed against DLL3, starting from said sample, using any suitable technique known in the field.

In another embodiment, such naturally occurring VHH domains against DLL3, are obtained from naïve libraries of Camelid VHH sequences, for example by screening such a library using DLL3, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against DLL3, involves suitably immunizing a transgenic mammal that is capable of expressing heavy chain antibodies (i.e., so as to raise an immune response and/or heavy chain antibodies directed against DLL3), obtaining a suitable biological sample from said transgenic mammal (such as a blood sample, serum sample or sample of B-cells), and then generating VHH sequences directed against DLL3, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-DLL3 single domain antibody of the DLL3 targeting immune cell engaging protein comprises a single domain antibody with an amino acid sequence that corresponds to the amino acid sequence of a naturally occurring VHH domain, but that has been “humanized”, i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining the anti-DLL3 single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-DLL3 single domain antibody of the disclosure or a nucleotide sequence or nucleic acid encoding the same.

In some embodiments, the DLL3 binding domain is an anti-DLL3 specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the DLL3 binding domain comprises any domain that binds to DLL3 including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the DLL3 binding domain is a single domain antibody. In some embodiments, the anti-DLL3 single domain antibody comprises heavy chain variable complementarity determining regions (CDR), CDR1, CDR2, and CDR3.

In some embodiments, the DLL3 binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the DLL3 binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the DLL3 binding domain comprises an amino acid sequence selected from the group consisting of SEQ ID NOS: 1308-1750. In some embodiments, CDR1 of the DLL3 binding domain comprises a sequence selected from the group consisting of SEQ ID NOS: 1751-2193, or one or more amino acid substitutions relative to a sequence selected from the group consisting of SEQ ID NOS: 1751-2193. In some embodiments, CDR2 comprises a sequence selected from the group consisting of SEQ ID NOS: 2194-2636, or one or more amino acid substitutions relative to a sequence selected from the group consisting of SEQ ID NOS: 2194-2636. In some embodiments, the CDR3 comprises a sequence selected from the group consisting of SEQ ID NOS: 2637-3080, or one or more substitutions relative to a sequence selected from the group consisting of SEQ ID NOS: 2637-3080.

In some embodiments, the CDR1 comprises an amino acid sequence selected from the group consisting of SEQ ID NOS: 1751-2193 or a variant having one, two, three, four, five, six, seven, eight, nine, or ten amino acid substitutions in an amino acid selected from the group consisting of SEQ ID NOS: 1751-2193. In some embodiments, the CDR2 comprises an amino acid sequence selected from the group consisting of SEQ ID NOS: 2194-2636 or a variant having one, two, three, four, five, six, seven, eight, nine, or ten amino acid substitutions in an amino acid sequence selected from the group consisting of SEQ ID NOS: 2194-2636. In some embodiments, the CDR3 comprises an amino acid sequence selected from the group consisting of SEQ ID NOS: 2637-3080 or a variant having one, two, three, four, five, six, seven, eight, nine, or ten amino acid substitutions in a sequence selected from the group consisting of SEQ ID NOS: 2637-3080.

In some embodiments, the CDR1 comprises an amino acid sequence selected from the group consisting of SEQ ID NOS: 1803-1836 or a variant having one, two, three, four, five, six, seven, eight, nine, or ten amino acid substitutions in an amino acid selected from the group consisting of SEQ ID NOS: 1803-1836. In some embodiments, the CDR2 comprises an amino acid sequence selected from the group consisting of SEQ ID NOS: 2246-2279 or a variant having one, two, three, four, five, six, seven, eight, nine, or ten amino acid substitutions in an amino acid sequence selected from the group consisting of SEQ ID NOS: 2246-2279. In some embodiments, the CDR3 comprises an amino acid sequence selected from the group consisting of SEQ ID NOS: 2689-2722 or a variant having one, two, three, four, five, six, seven, eight, nine, or ten amino acid substitutions in a sequence selected from the group consisting of SEQ ID NOS: 2689-2722.

In some embodiments, the CDR1 comprises an amino acid sequence selected from the group consisting of SEQ ID NOS: 1837-2117 or a variant having one, two, three, four, five, six, seven, eight, nine, or ten amino acid substitutions in an amino acid selected from the group consisting of SEQ ID NOS: 1837-2117. In some embodiments, the CDR2 comprises an amino acid sequence selected from the group consisting of SEQ ID NOS: 2280-2560 or a variant having one, two, three, four, five, six, seven, eight, nine, or ten amino acid substitutions in an amino acid sequence selected from the group consisting of SEQ ID NOS: 2280-2560. In some embodiments, the CDR3 comprises an amino acid sequence selected from the group consisting of SEQ ID NOS: 2723-3003 or a variant having one, two, three, four, five, six, seven, eight, nine, or ten amino acid substitutions in a sequence selected from the group consisting of SEQ ID NOS: 2723-3003.

In some embodiments, the CDR1 comprises an amino acid sequence selected from the group consisting of SEQ ID NOS: 2118-2193 or a variant having one, two, three, four, five, six, seven, eight, nine, or ten amino acid substitutions in an amino acid selected from the group consisting of SEQ ID NOS: 2118-2193. In some embodiments, the CDR2 comprises an amino acid sequence selected from the group consisting of SEQ ID NOS: 2561-2636 or a variant having one, two, three, four, five, six, seven, eight, nine, or ten amino acid substitutions in an amino acid sequence selected from the group consisting of SEQ ID NOS: 2561-2636. In some embodiments, the CDR3 comprises an amino acid sequence selected from the group consisting of SEQ ID NOS: 3004-3080 or a variant having one, two, three, four, five, six, seven, eight, nine, or ten amino acid substitutions in a sequence selected from the group consisting of SEQ ID NOS: 3004-3080.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 1308-1750. In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 1360-1393.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 1394-1674.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to SEQ ID NO:1375, or a sequence derived from SEQ ID NO:1375.

In various embodiments, the DLL3 binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to SEQ ID NO:1382, or a sequence derived from SEQ ID NO:1382.

In some embodiments, the DLL3 binding domain of the DLL3 targeting immune cell engaging protein is cross-reactive with human and cynomolgus DLL3. In some embodiments, the DLL3 binding domain is specific for human DLL3. In certain embodiments, the DLL3 binding domain disclosed herein binds to human DLL3 with a human Kd (hKd). In certain embodiments, the DLL3 binding domain disclosed herein binds to cynomolgus DLL3 with a cynomolgus Kd (cKd). In certain embodiments, the DLL3 binding domain disclosed herein binds to both cynomolgus DLL3 and a human DLL3, with a cyno Kd (cKd) and a human Kd, respectively (hKd). In some embodiments, the DLL3 binding protein binds to human and cynomolgus DLL3 with comparable binding affinities (i.e., hKd and cKd values do not differ by more than ±10%). In some embodiments, the hKd and the cKd range from about 0.001 nM to about 500 nM. In some embodiments, the hKd and the cKd range from about 0.001 nM to about 450 nM. In some embodiments, the hKd and the cKd range from about 0.001 nM to about 400 nM. In some embodiments, the hKd and the cKd range from about 0.001 nM to about 350 nM. In some embodiments, the hKd and the cKd range from about 0.001 nM to about 300 nM. In some embodiments, the hKd and the cKd range from about 0.001 nM to about 250 nM. In some embodiments, the hKd and the cKd range from about 0.001 nM to about 200 nM. In some embodiments, the hKd and the cKd range from about 0.001 nM to about 150 nM. In some embodiments, the hKd and the cKd range from about 0.001 nM to about 100 nM. In some embodiments, the hKd and the cKd range from about 0.1 nM to about 90 nM. In some embodiments, the hKd and the cKd range from about 0.2 nM to about 80 nM. In some embodiments, the hKd and the cKd range from about 0.3 nM to about 70 nM. In some embodiments, the hKd and the cKd range from about 0.4 nM to about 50 nM. In some embodiments, the hKd and the cKd range from about 0.5 nM to about 30 nM. In some embodiments, the hKd and the cKd range from about 0.6 nM to about 10 nM. In some embodiments, the hKd and the cKd range from about 0.7 nM to about 8 nM. In some embodiments, the hKd and the cKd range from about 0.8 nM to about 6 nM. In some embodiments, the hKd and the cKd range from about 0.9 nM to about 4 nM. In some embodiments, the hKd and the cKd range from about 1 nM to about 2 nM.

In certain embodiments, the DLL3 binding domains of the present disclosure preferentially bind membrane bound DLL3 over soluble DLL3. Membrane bound DLL3 refers to the presence of DLL3 in or on the cell membrane surface of a cell that expresses DLL3. Soluble DLL3 refers to DLL3 that is no longer on in or on the cell membrane surface of a cell that expresses or expressed DLL3. In certain instances, the soluble DLL3 is present in the blood and/or lymphatic circulation in a subject. In one embodiment, the DLL3 binding proteins bind membrane-bound DLL3 at least 5 fold, 10 fold, 15 fold, 20 fold, 25 fold, 30 fold, 40 fold, 50 fold, 100 fold, 500 fold, or 1000 fold greater than soluble DLL3. In one embodiment, the antigen binding proteins of the present disclosure preferentially bind membrane-bound DLL3 30 fold greater than soluble DLL3. Determining the preferential binding of an antigen binding protein to membrane bound DLL3 over soluble DLL3 can be readily determined using assays well known in the art.

In some embodiments, any of the foregoing DLL3 binding domains (e.g., anti-DLL3 single domain antibodies of SEQ ID NOS: 1308-1750) are affinity peptide tagged for ease of purification. In some embodiments, the affinity peptide tag is six consecutive histidine residues, also referred to as 6X-his (SEQ ID NO: 3503).

In some embodiments, any of the foregoing DLL3 binding domains (e.g., anti-DLL3 single domain antibodies of SEQ ID NOS: 1308-1750) are affinity peptide tagged for ease of purification. In some embodiments, the affinity peptide tag is six consecutive histidine residues, also referred to as 6X-his (SEQ ID NO: 3503).

### Epidermal Growth Factor Receptor (EGFR) Binding Proteins

Epidermal growth factor receptor (EGFR) has been causally implicated in human malignancy. Abnormal activity of the Her family of receptors is involved with breast cancer. EGFR, Her-3, and Her-4 are frequently expressed in ovarian granulosa cell tumors (Leibl, S. et al., Gynecol Oncol 101:18-23 (2005). In particular, increased expression of EGFR has been observed in breast, bladder, lung, head, neck and stomach cancer as well as glioblastomas.

Increased EGFR receptor expression may be associated with increased production of a EGFR ligand, transforming growth factor alpha (TGF-α), by the same tumor cells resulting in receptor activation by an autocrine stimulatory pathway.

Cetuximab (Erbitux™), an anti-EGFR antibody, has been associated with potentially life threatening infusion reactions (Thomas, M., Clin J Oncol Nurs. 9(3):332-8 (2005)). Gefitinib (Iressa™) and erlotinib (Tarceva™), both EGFR specific small molecule inhibitors, are associated with a risk of interstitial lung disease (Sandler A, Oncology 20(5 Suppl 2):35-40 (2006)). Individual patients may be predisposed to particular types of complications that affect the choice of drug treatment. There is a need for a greater choice of treatment options which allows physicians to select the therapeutic with the best side effect profile for an individual patient. The present disclosure provides novel polypeptides and protein therapeutics useful in methods of treatment, particularly for treatment of conditions associated with abnormal expression of EGFR.

Epidermal growth factor receptor (EGFR, also known as HER1 or ErbB1) is a member of the ErbB/HER family of type 1 receptor tyrosine kinases (RTKs). Other members of this family include ErbB2 (HER2 or Neu), ErbB3 (HER3) and ErbB4 (HER4). Known ligands for EGFR include epidermal growth factor (EGF) and transforming growth factor alpha (TGF-alpha). Ligand binding to EGFR is known to induce tyrosine phosphorylation and receptor dimerization with other ErbB family members.

RTKs such as EGFR function to allow cells to respond to diverse external stimuli. However, aberrant activation and/or overexpression of EGFR is associated with the development and progression of several human cancers. Accordingly, EGFR is a target for anti-cancer therapies. Approved drugs targeting EGFR include small molecule inhibitors such as gefitinib (Iressa®) and erlotinib (Tarceva®), and anti-EGFR antibodies such as cetuximab (Erbitux®) and panitumumab (Vectibix®). Anti-EGFR antibodies are mentioned in, e.g., U.S. Pat. Nos. 4,943,533, 5,844,093, 7,060,808, 7,247,301, 7,595,378, 7,723,484, and 7,939,072. There is still a need for having available further options for the treatment of diseases related to the overexpression of EGFR, including, but not limited to, renal cell carcinoma, pancreatic carcinoma, breast cancer, head and neck cancer, prostate cancer, malignant gliomas, osteosarcoma, colorectal cancer, gastric cancer (e.g., gastric cancer with MET amplification), malignant mesothelioma, multiple myeloma, ovarian cancer, small cell lung cancer, non-small cell lung cancer (e.g., EGFR-dependent non-small cell lung cancer), synovial sarcoma, thyroid cancer, or melanoma. The present disclosure provides, in certain embodiments, EGFR binding proteins, EGFR targeting immune cell engaging proteins containing EGFR binding domains which specifically bind to EGFR on the surface of tumor target cells.

The design of the EGFR targeting immune cell engaging proteins described herein allows the binding domain to EGFR to be flexible in that the binding domain to EGFR can be any type of binding domain, including but not limited to, domains from a monoclonal antibody, a polyclonal antibody, a recombinant antibody, a human antibody, a humanized antibody. In some embodiments, the binding domain to EGFR is a single chain variable fragments (scFv), single-domain antibody such as a heavy chain variable domain (VH), a light chain variable domain (VL) and a variable domain (VHH) of camelid derived single domain antibody. In other embodiments, the binding domain to EGFR is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to EGFR is a ligand or peptide that binds to or associates with EGFR. In yet further embodiments, the binding domain to EGFR is a knottin. In yet further embodiments, the binding domain to EGFR is a small molecular entity.

In some embodiments, the EGFR binding domain is an anti-EGFR antibody or an antibody variant. As used herein, the term “antibody variant” refers to variants and derivatives of an antibody described herein. In certain embodiments, amino acid sequence variants of the anti-EGFR antibodies described herein are contemplated. For example, in certain embodiments amino acid sequence variants of anti-EGFR antibodies described herein are contemplated to improve the binding affinity and/or other biological properties of the antibodies. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to arrive at the final construct, provided that the final construct possesses the desired characteristics, e.g., antigen-binding. In certain embodiments, antibody variants having one or more amino acid substitutions are provided. Sites of interest for substitution mutagenesis include the CDRs and framework regions. Examples of such substitutions are described below. Amino acid substitutions may be introduced into an antibody of interest and the products screened for a desired activity, e.g., retained/improved antigen binding, decreased immunogenicity, or improved T-cell mediated cytotoxicity (TDCC). Both conservative and non-conservative amino acid substitutions are contemplated for preparing the antibody variants.

In another example of a substitution to create a variant anti-EGFR antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding.

In some embodiments, the EGFR binding domain of the EGFR targeting immune cell engaging protein is a single domain antibody such as a heavy chain variable domain (VH), a variable domain (VHH) of a llama derived sdAb, a peptide, a ligand or a small molecule entity specific for EGFR. In some embodiments, the EGFR binding domain of the EGFR targeting immune cell engaging protein described herein is any domain that binds to EGFR including but not limited to domains from a monoclonal antibody, a polyclonal antibody, a recombinant antibody, a human antibody, a humanized antibody. In certain embodiments, the EGFR binding domain is a single-domain antibody. In other embodiments, the EGFR binding domain is a peptide. In further embodiments, the EGFR binding domain is a small molecule.

In one embodiment, a single domain antibody corresponds to the VHH domains of naturally occurring heavy chain antibodies directed against EGFR. As further described herein, such VHH sequences can generally be generated or obtained by suitably immunizing a species of Llama with EGFR, (i.e., so as to raise an immune response and/or heavy chain antibodies directed against EGFR), by obtaining a suitable biological sample from said Llama (such as a blood sample, serum sample or sample of B-cells), and by generating VHH sequences directed against EGFR, starting from said sample, using any suitable technique known in the field.

In another embodiment, such naturally occurring VHH domains against EGFR, are obtained from naïve libraries of Camelid VHH sequences, for example by screening such a library using EGFR, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against EGFR, involves suitably immunizing a transgenic mammal that is capable of expressing heavy chain antibodies (i.e., so as to raise an immune response and/or heavy chain antibodies directed against EGFR), obtaining a suitable biological sample from said transgenic mammal (such as a blood sample, serum sample or sample of B-cells), and then generating VHH sequences directed against EGFR, starting from said sample, using any suitable technique known in the field. For example, for this purpose, the heavy chain antibody-expressing rats or mice and the further methods and techniques described in WO 02/085945 and in WO 04/049794 can be used.

In some embodiments, the EGFR binding domain of this disclosure binds to a protein comprising the sequence of SEQ ID NO: 3205 (UniProt Accession No. Q504U8). In some embodiments, the EGFR binding domain binds to a protein comprising a truncated sequence compared to SEQ ID NO: 3205. In some embodiments, the EGFR binding domain binds to a protein comprising the sequence of SEQ ID NO: 3206 (UniProt Accession No. Q01279). In some embodiments, the EGFR binding domain binds to a protein comprising a truncated sequence compared to SEQ ID NO: 3206. In some embodiments, the EGFR binding domain binds to a protein comprising the sequence of SEQ ID NO: 3207 (UniProt Accession No. A0A2K5WK39). In some embodiments, the EGFR binding domain binds to a protein comprising a truncated sequence compared to SEQ ID NO: 3207.

In some embodiments, the EGFR binding domains disclosed herein recognize full-length EGFR. In certain instances, the EGFR binding domains disclosed herein recognize an epitope within EGFR, such as, in some cases the EGFR targeting immune cell engaging proteins interact with one or more amino acids found within the extracellular domain of human EGFR (e.g., within extracellular domain I, II, III, and/or IV). The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within the extracellular domain of EGFR. Alternatively, the epitope may consist of a plurality of non-contiguous amino acids (or amino acid sequences) located within the extracellular domain of EGFR.

In some embodiments, the EGFR binding proteins of this disclosure binds to the full length EGFR protein or to a fragment thereof, such as epitope containing fragments within the full length EGFR protein, as described above. In some cases, the epitope containing fragment comprises antigenic or immunogenic fragments and derivatives thereof of the EGFR protein. Epitope containing fragments, including antigenic or immunogenic fragments, are, in some embodiments, 12 amino acids or more, e.g., 20 amino acids or more, 50 or 100 amino acids or more. The EGFR fragments, in some embodiments, comprises 95% or more of the length of the full protein, 90% or more, 75% or 50% or 25% or 10% or more of the length of the full protein. In some embodiments, the epitope-containing fragments of EGFR including antigenic or immunogenic fragments are capable of eliciting a relevant immune response in a patient. Derivatives of EGFR include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the EGFR sequence provided in SEQ ID NO: 3205, 3206, or 3207.

In some embodiments, substitutions comprise conservative substitutions. Derivatives and variants of, in some examples, have essentially the same biological function as the protein from which they are derived. For instance, derivatives and variants of EGFR are, in some cases, comparably antigenic or immunogenic to the protein from which they are derived, have either the ligand-binding activity, or the active receptor-complex forming ability, or preferably both, of the protein from which they are derived, and have the same tissue distribution as EGFR.

In some embodiments, the EGFR binding protein specifically binds EGFR with equivalent or better affinity as that of a reference EGFR binding protein, and the EGFR binding protein in such embodiments comprises an affinity matured EGFR binding molecule, and is derived from the EGFR binding parental molecule, comprising one or more amino acid mutations (e.g., a stabilizing mutation, a destabilizing mutation) with respect to the EGFR binding parental molecule. In some embodiments, the affinity matured EGFR binding molecule has superior stability with respect to selected destabilizing agents, as that of a reference EGFR binding parental molecule. In some embodiments, the affinity matured EGFR binding molecule is identified in a process comprising panning of one or more pre-candidate EGFR binding molecules derived from one or more EGFR binding parental molecule, expressed in a phage display library, against a EGFR protein, such as a human EGFR protein. The pre-candidate EGFR binding molecule comprises, in some embodiments, amino acid substitutions in the variable regions, CDRs, or framework residues, relative to a parental molecule.

As used herein, “Phage display” refers to a technique by which variant polypeptides are displayed as fusion proteins to at least a portion of a coat protein on the surface of phage, e.g., filamentous phage, particles. A utility of phage display lies in the fact that large libraries of randomized protein variants can be rapidly and efficiently selected for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol, 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that selection is on the basis of intrinsic ligand affinity, and phagemid vectors are used, which simplify DNA manipulations. Lowman and Wells, Methods: A companion to Methods in Enzymology, 3:205-0216 (1991).

In some embodiments, the panning comprises using varying binding times and concentrations to identify EGFR binding molecules with increased or decreased on-rates, from pre-candidate EGFR binding molecules. In some embodiments, the panning comprises using varying wash times to identify EGFR binding molecules with increased or decreased off-rates, from pre-candidate EGFR molecules. In some embodiments, the panning comprises using both varying binding times and varying wash times. In some embodiments, one or more stabilizing mutations are combined to increase the stability of the affinity matured EGFR binding molecule, for example, by shuffling to create a second-stage combinatorial library from such mutants and conducting a second round of panning followed by a binding selection.

In some embodiments, the affinity matured EGFR binding molecule comprises an equivalent or better affinity to an EGFR protein (such as human EGFR protein) as that of a EGFR binding parental molecule, but that has reduced cross reactivity, or in some embodiments, increased cross reactivity, with selected substances, such as ligands, proteins, antigens, or the like, other than the EGFR epitope for which the EGFR binding parental molecule is specific, or is designed to be specific for. In regard to the latter, an affinity matured EGFR binding molecule, in some embodiments, is more successfully tested in animal models if the affinity matured EGFR binding molecule is reacted with both human EGFR and the corresponding target of the animal model, e.g. mouse EGFR or cynomolgus EGFR. In some embodiments, the parental EGFR binding molecule binds to human EGFR with an affinity of about 10 nM or less, and to cynomolgus EGFR with an affinity of about 15 nM or less. In some embodiments, the affinity matured EGFR binding molecule, identified after one round of panning, binds to human EGFR with an affinity of about 5 nM or less, and to cynomolgus EGFR with an affinity of about 7.5 nM or less. In some embodiments, the affinity matured EGFR binding molecule, identified after two rounds of panning, binds to human EGFR with an affinity of about 2.5 nM or less, and to cynomolgus EGFR with an affinity of about 3.5 nM or less.

In some embodiments, the EGFR binding protein comprises an antigen-specific binding domain polypeptide that specifically bind to targets, such as targets on diseased cells, or targets on other cells that support the diseased state, such as targets on stromal cells that support tumor growth or targets on immune cells that support disease-mediated immunosuppression. In some examples, the antigen-specific binding domain includes antibodies, single chain antibodies, Fabs, Fv, T-cell receptor binding domains, ligand binding domains, receptor binding domains, domain antibodies, single domain antibodies, minibodies, nanobodies, peptibodies, or various other antibody mimics (such as affimers, affitins, alphabodies, atrimers, CTLA4-based molecules, adnectins, anticalins, Kunitz domain-based proteins, avimers, knottins, fynomers, darpins, affibodies, affilins, monobodies and armadillo repeat protein-based proteins).

In some embodiments, an anti-EGFR single domain antibody of the EGFR targeting immune cell engaging protein comprises a single domain antibody with an amino acid sequence that corresponds to the amino acid sequence of a naturally occurring VHH domain, but that has been “humanized,” i.e., by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining the anti-EGFR single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-EGFR single domain antibody of the disclosure or a nucleotide sequence or nucleic acid encoding the same.

In some embodiments, the EGFR binding domain is an anti-EGFR specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the EGFR binding domain comprises any domain that binds to EGFR including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the EGFR binding domain is a single domain antibody. In some embodiments, the anti-EGFR single domain antibody comprises heavy chain variable complementarity determining regions (CDR), CDR1, CDR2, and CDR3.

In some embodiments, the EGFR binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the EGFR binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the EGFR binding domain comprises an amino acid sequence selected from the group consisting of SEQ ID NOS: 798-846.

In some embodiments, the EGFR binding domains described herein comprise a polypeptide having a sequence selected from the group consisting of SEQ ID NOS: 798-846, subsequences thereof, and variants thereof. In some embodiments, the EGFR binding protein comprises at least 70%-95% or more homology to a sequence selected from the group consisting of SEQ ID NOS: 798-846, subsequences thereof, and variants thereof. In some embodiments, the EGFR binding protein comprises at least 70%, 75%, 80%, 85%, 90%, 95%, 96%, 97%, 98%, 99%, or more homology to a sequence selected from the group consisting of SEQ ID NOS: 798-846, subsequences thereof, and variants thereof. In some embodiments, the EGFR binding protein comprises at least 70%-95% or more identity to a sequence selected from the group consisting of SEQ ID NOS: 798-846, subsequences thereof, and variants thereof. In some embodiments, the EGFR binding protein comprises at least 70%, 75%, 80%, 85%, 90%, 95%, 96%, 97%, 98%, 99% or more identity to a sequence selected from the group consisting of SEQ ID NOS: 798-846, subsequences thereof, and variants thereof.

In some embodiments, the CDR1 comprises the amino acid sequence as set forth in any one of SEQ ID NOS: 651-699 or a variant having one, two, three, four, five, six, seven, eight, nine, or ten amino acid substitutions in any one of SEQ ID NOS: 651-699. In some embodiments, the CDR2 comprises a sequence as set forth in any one of SEQ ID NOS: 700-748 or a variant having one, two, three, four, five, six, seven, eight, nine, or ten amino acid substitutions in any one of SEQ ID NOS: 700-748. In some embodiments, the CDR3 comprises a sequence as set forth in any one of SEQ ID NOS: 148-196 or a variant having one, two, three, four, five, six, seven, eight, nine, or ten amino acid substitutions in any one of SEQ ID NOS: 749-797.

In various embodiments, the EGFR binding domain of the present disclosure is at least about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 798-846.

In various embodiments, a complementarity determining region of the EGFR binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence set forth in any one of SEQ ID NOS: 651-699.

In various embodiments, a complementarity determining region of the EGFR binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence set forth in any one of SEQ ID NOS: 700-748.

In various embodiments, a complementarity determining region of the EGFR binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence set forth in any one of SEQ ID NOS: 749-797.

In some embodiments, the EGFR binding domain is cross-reactive with human cynomolgus and mouse EGFR. In some embodiments, the EGFR binding domain is specific for human EGFR. In certain embodiments, the EGFR binding domains disclosed herein bind to human EGFR with a human Kd (hKd). In certain embodiments, the EGFR binding domains disclosed herein bind to cynomolgus EGFR with a cyno Kd (cKd). In certain embodiments, the EGFR binding domains disclosed herein bind to cynomolgus EGFR with a mouse Kd (mKd). In certain embodiments, the EGFR binding domains disclosed herein bind to both cynomolgus EGFR and a human EGFR, with a cyno Kd (cKd) and a human Kd (hKd), respectively. In certain embodiments, the EGFR binding domains disclosed herein bind to cynomolgus EGFR, mouse EGFR, and a human EGFR, with a cyno Kd (cKd), mouse Kd (mKd), and a human Kd (hKd), respectively. In some embodiments, the EGFR binding protein binds to human, mouse and cynomolgus EGFR with comparable binding affinities (i.e., hKd, mKd and cKd values do not differ by more than ±10%). In some embodiments, the hKd, mKd and the cKd range from about 0.1 nM to about 500 nM. In some embodiments, the hKd, mKd and the cKd range from about 0.1 nM to about 450 nM. In some embodiments, the hKd, mKd and the cKd range from about 0.1 nM to about 400 nM. In some embodiments, the hKd, mKd and the cKd range from about 0.1 nM to about 350 nM. In some embodiments, the hKd, mKd and the cKd range from about 0.1 nM to about 300 nM. In some embodiments, the hKd, mKd and the cKd range from about 0.1 nM to about 250 nM. In some embodiments, the hKd, mKd and the cKd range from about 0.1 nM to about 200 nM. In some embodiments, the hKd, mKd and the cKd range from about 0.1 nM to about 150 nM. In some embodiments, the hKd, mKd and the cKd range from about 0.1 nM to about 100 nM. In some embodiments, the hKd, mKd and the cKd range from about 0.1 nM to about 90 nM. In some embodiments, the hKd, mKd and the cKd range from about 0.2 nM to about 80 nM. In some embodiments, the hKd, mKd and the cKd range from about 0.3 nM to about 70 nM. In some embodiments, the hKd, mKd and the cKd range from about 0.4 nM to about 50 nM. In some embodiments, the hKd, mKd and the cKd range from about 0.5 nM to about 30 nM. In some embodiments, the hKd, mKd and the cKd range from about 0.6 nM to about 10 nM. In some embodiments, the hKd, mKd and the cKd range from about 0.7 nM to about 8 nM. In some embodiments, the hKd, mKd and the cKd range from about 0.8 nM to about 6 nM. In some embodiments, the hKd, mKd and the cKd range from about 0.9 nM to about 4 nM. In some embodiments, the hKd, mKd and the cKd range from about 1 nM to about 2 nM.

In some embodiments, any of the foregoing EGFR binding domains (e.g., anti-EGFR single domain antibodies of SEQ ID NOS: 798-846) are affinity peptide tagged for ease of purification. In some embodiments, the affinity peptide tag is six consecutive histidine residues, also referred to as 6X-His (SEQ ID NO: 3503).

In certain embodiments, the EGFR binding domains of the present disclosure preferentially bind membrane bound EGFR over soluble EGFR Membrane bound EGFR refers to the presence of EGFR in or on the cell membrane surface of a cell that expresses EGFR. Soluble EGFR refers to EGFR that is no longer on in or on the cell membrane surface of a cell that expresses or expressed EGFR. In certain instances, the soluble EGFR is present in the blood and/or lymphatic circulation in a subject. In one embodiment, the EGFR binding domains bind membrane-bound EGFR at least 5 fold, 10 fold, 15 fold, 20 fold, 25 fold, 30 fold, 40 fold, 50 fold, 100 fold, 500 fold, or 1000 fold greater than soluble EGFR. In one embodiment, the EGFR targeting immune cell engaging proteins of the present disclosure preferentially bind membrane-bound EGFR 30 fold greater than soluble EGFR. Determining the preferential binding of an antigen binding protein to membrane bound EGFR over soluble EGFR can be readily determined using binding assays. It is contemplated that in some embodiments the EGFR binding protein is fairly small and no more than 25 kDa, no more than 20 kDa, no more than 15 kDa, or no more than 10 kDa in some embodiments. In certain instances, the EGFR binding protein is 5 kDa or less if it is a peptide or small molecule entity.

In other embodiments, the EGFR binding proteins described herein comprise small molecule entity (SME) binders for EGFR. SME binders are small molecules averaging about 500 to 2000 Da in size and are attached to the EGFR binding proteins by known methods, such as sortase ligation or conjugation. In these instances, the EGFR binding protein comprises a domain comprising a sortase recognition sequence, e.g., LPETG (SEQ ID NO: 3200). To attach a SME binder to EGFR binding protein comprising a sortase recognition sequence, the protein is incubated with a sortase and a SME binder whereby the sortase attaches the SME binder to the recognition sequence. In yet other embodiments, the EGFR binding proteins described herein comprise a knottin peptide for binding EGFR. Knottins are disufide-stabilized peptides with a cysteine knot scaffold and have average sizes about 3.5 kDa. Knottins have been contemplated for binding to certain tumor molecules such as EGFR. In further embodiments, the EGFR binding proteins described herein comprise a natural EGFR ligand.

In some embodiments, the EGFR binding protein comprises more than one domain and are of a single-polypeptide design with flexible linkage of the domains. This allows for facile production and manufacturing of the EGFR binding proteins as they can be encoded by single cDNA molecule to be easily incorporated into a vector. Further, in some embodiments where the EGFR binding proteins described herein are a monomeric single polypeptide chain, there are no chain pairing issues or a requirement for dimerization. It is contemplated that, in such embodiments, the EGFR binding proteins described herein have a reduced tendency to aggregate.

In the EGFR binding proteins comprising more than one domain, the domains are linked by one or more internal linker. In certain embodiments, the internal linkers are “short,” i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, the internal linkers are “long,” i.e., consist of 15, 20 or 25 amino acid residues. In some embodiments, the internal linkers consist of about 3 to about 15, for example 8, 9 or 10 contiguous amino acid residues. Regarding the amino acid composition of the internal linkers, peptides are selected with properties that confer flexibility to the EGFR binding proteins, do not interfere with the binding domains as well as resist cleavage from proteases. For example, glycine and serine residues generally provide protease resistance. Examples of internal linkers suitable for linking the domains in the EGFR binding proteins include but are not limited to (GS)ₙ (SEQ ID NO: 3525), (GGS)ₙ(SEQ ID NO: 3526), (GGGS)ₙ (SEQ ID NO: 3527), (GGSG)ₙ (SEQ ID NO: 3528), (GGSGG)ₙ (SEQ ID NO: 3194), (GGGGS)ₙ(SEQ ID NO: 3195), (GGGGG)ₙ(SEQ ID NO: 3196), or (GGG)ₙ (SEQ ID NO: 3197), wherein n is 1, 2, 3, 4, 5, 6, 7, 8, 9, or 10. In one embodiment, the linker is (GGGGSGGGGSGGGGSGGGGS)(SEQ ID NO: 3198), (GGGGSGGGGSGGGGS) (SEQ ID NO: 3199), or (GGGGSGGGS) (SEQ ID NO: 3504).

In some cases, where the EGFR binding protein comprises more than one domain, the domains within the EGFR binding proteins are conjugated using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See, e.g., Pavel Strp, Veracity of microbial transglutaminase, Bioconjugate Chem. 25, 5, 855-862).

In some examples are provided EGFR binding proteins comprising more than one domain, wherein one of the domains comprises an acceptor glutamine in a constant region, which can then be conjugated to another domain via a lysine-based linker (e.g., any primary amine chain which is a substrate for TGase, e.g. comprising an alkylamine, oxoamine) wherein the conjugation occurs exclusively on one or more acceptor glutamine residues present in the targeting moiety outside of the antigen combining site (e.g., outside a variable region, in a constant region). Conjugation thus does not occur on a glutamine, e.g. an at least partly surface exposed glutamine, within the variable region. The EGFR binding protein, in some examples, is formed by reacting one of the domains with a lysine-based linker in the presence of a TGase.

In some embodiments, where one or more domains within the EGFR binding proteins are directly joined, a hybrid vector is made where the DNA encoding the directly joined domains are themselves directly ligated to each other. In some embodiments, where linkers are used, a hybrid vector is made where the DNA encoding one domain is ligated to the DNA encoding one end of a linker moiety and the DNA encoding another domain is ligated to the other end of the linker moiety.

In some embodiments, the EGFR binding protein is a single chain variable fragments (scFv), single-domain antibody such as a heavy chain variable domain (VH), a light chain variable domain (VL) and a variable domain (VHH) of camelid derived single domain antibody. In other embodiments, the EGFR binding protein is a non-Ig binding domain, i.e., an antibody mimetic, such as anticalins, affilins, affibody molecules, affimers, affitins, alphabodies, avimers, DARPins, fynomers, kunitz domain peptides, and monobodies. In further embodiments, the EGFR binding protein is a ligand or peptide that binds to or associates with EGFR. In yet further embodiments, the EGFR binding protein is a knottin. In yet further embodiments, the binding domain to EGFR is a small molecular entity.

In some embodiments, the EGFR binding proteins as set forth above are fused to an Fc region from any species, including but not limited to, human immunoglobulin, such as human IgG1, a human IgG2, a human IgG3, human IgG4, to generate Fc-fusion proteins. In some embodiments, the Fc-fusion proteins of this disclosure have extended half-life compared to an otherwise identical EGFR binding protein. In some embodiments, the Fc-fusion EGFR binding proteins of this disclosure contain inter alia one or more additional amino acid residue substitutions, mutations and/or modifications, e.g., in the Fc region, which result in a binding protein with preferred characteristics including, but not limited to: altered pharmacokinetics, extended serum half-life.

In some embodiments, such Fc-fused EGFR binding proteins provide extended half-lives in a mammal, such as in a human, of greater than 5 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-life, in some cases, results in a higher serum titer which thus reduces the frequency of the administration of the EGFR binding proteins and/or reduces the concentration of the antibodies to be administered. Binding to human FcRn in vivo and serum half-life of human FcRn high affinity binding polypeptides is assayed, in some examples, in transgenic mice or transfected human cell lines expressing human FcRn, or in primates to which the polypeptides with a variant Fc region are administered.

The EGFR binding proteins, in some cases, are differentially modified during or after production, e.g., by glycosylation, acetylation, phosphorylation, amidation, derivatization by known protecting/blocking groups, proteolytic cleavage, linkage to an antibody molecule or other cellular ligand, etc. Any of numerous chemical modifications are carried out by techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

Various post-translational modifications of the EGFR binding proteins also encompassed by the disclosure include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. Moreover, the EGFR binding proteins are, in some cases, modified with a detectable label, such as an enzymatic, fluorescent, radioisotopic or affinity label to allow for detection and isolation of the modulator.

In some embodiments, the EGFR binding proteins of the disclosure are monovalent or multivalent bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen).

### FLT3 Binding Proteins

FLT3, also known as fetal liver kinase 2 (FLK-2), stem cell tyrosine kinase 1 (STK-1) and CD135, is a member of the class III receptor tyrosine kinases. Normally, FLT3 is expressed on immature myeloid-lymphocytic precursor cells and dendritic cell precursors, but rarely on mature adult cells. FLT3 is overexpressed in approximately 90% of acute myeloid leukemia (AML), a majority of acute lymphocytic leukemia (ALL) and the blast-crisis phase of chronic myeloid leukemia (BC-CML). Stimulation by FLT3 ligand (FL) enhances the proliferation and survival of leukemia cells. Inhibition of FLT3 signaling leads to apoptosis in dendritic cells and inhibition of immune responses. The MAPK, PI3K and Stat5 pathways have been identified to be involved in the downstream signaling of activated FLT3 (See e.g., Stirewalt D L and J P, Radich, J P. Nat Rev Cancer 3:650-665 (2003)).

Described herein are immune cell engaging proteins that comprise an FLT3 binding domain, pharmaceutical compositions thereof, as well as nucleic acids, recombinant expression vectors and host cells for making such proteins thereof. Also provided are methods of using the disclosed proteins comprising an FLT3 binding domain of this disclosure, in the prevention, and/or treatment of diseases, conditions and disorders. In some embodiments, an FLT3 binding domain of this disclosure inhibits FL-induced phosphorylation of wild-type FLT3 and downstream kinases of MPK, PI3K, and STATS pathways in a disease such as leukemia. In some embodiments, an FLT3 binding domain of this disclosure has an improved ability to activate downstream immune effector functions such as antibody dependent cellular cytotoxicity (ADCC).

In some embodiments, the FLT3 binding domain binds to a human FLT3 protein comprising a sequence as set forth in SEQ ID NO: 3215 (UniProt ID: P36888). In some embodiments, the FLT3 binding domain binds to a protein comprising a truncated sequence compared to SEQ ID NO: 3215 (UniProt ID: P36888).

In some embodiments, the FLT3 binding domains disclosed herein recognize full-length FLT3 (e.g., an FLT3 protein comprising the sequence of SEQ ID NO: 3215 (UniProt ID: P36888). In certain instances, the FLT3 binding domains disclosed herein recognize an epitope within FLT3, such as, in some cases the FLT3 binding proteins interact with one or more amino acids found within a domain of human FLT3. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within a domain of FLT3 (e.g., an FLT3 protein comprising the sequence of SEQ ID NO: 3215 (UniProt ID: P36888). Alternatively, the epitope may consist of a plurality of non-contiguous amino acids (or amino acid sequences) located within a domain of FLT3 (e.g., an FLT3 protein comprising the sequence of SEQ ID NO: 3215 (UniProt ID: P36888).

In some embodiments, the FLT3 binding proteins of this disclosure binds to the full length of an FLT3 protein or to a fragment thereof, such as epitope containing fragments within the full length FLT3 protein, as described above. In some cases, the epitope containing fragment comprises antigenic or immunogenic fragments and derivatives thereof of the FLT3 protein. Epitope containing fragments, including antigenic or immunogenic fragments, are, in some embodiments, 12 amino acids or more, e.g., 20 amino acids or more, 50 or 100 amino acids or more. The FLT3 fragments, in some embodiments, comprises 95% or more of the length of the full protein, 90% or more, 75% or 50% or 25% or 10% or more of the length of the full protein. In some embodiments, the epitope-containing fragments of FLT3 including antigenic or immunogenic fragments are capable of eliciting a relevant immune response in a patient. Derivatives of FLT3 include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the FLT3 sequence (e.g., an FLT3 protein comprising the sequence of SEQ ID NO: 3215 (UniProt ID: P36888).

In some embodiments, substitutions comprise conservative substitutions. Derivatives and variants of, in some examples, have essentially the same biological function as the protein from which they are derived. For instance, derivatives and variants of FLT3 are, in some cases, comparably antigenic or immunogenic to the protein from which they are derived, have either the ligand-binding activity, or the active receptor-complex forming ability, or preferably both, of the protein from which they are derived, and have the same tissue distribution as FLT3.

In some embodiments, the FLT3 binding protein specifically binds FLT3 with equivalent or better affinity as that of a reference FLT3 binding protein, and the FLT3 binding protein in such embodiments comprises an affinity matured FLT3 binding molecule, and is derived from the FLT3 binding parental molecule, comprising one or more amino acid mutations (e.g., a stabilizing mutation, a destabilizing mutation) with respect to the FLT3 binding parental molecule. In some embodiments, the affinity matured FLT3 binding molecule has superior stability with respect to selected destabilizing agents, as that of a reference FLT3 binding parental molecule. In some embodiments, the affinity matured FLT3 binding molecule is identified in a process comprising panning of one or more pre-candidate FLT3 binding molecules derived from one or more FLT3 binding parental molecule, expressed in a phage display library, against an FLT3 protein, such as a human FLT3 protein. The pre-candidate FLT3 binding molecule comprises, in some embodiments, amino acid substitutions in the variable regions, CDRs, or framework residues, relative to a parental molecule.

As used herein, “Phage display,” refers to a technique by which variant polypeptides are displayed as fusion proteins to at least a portion of a coat protein on the surface of phage, e.g., filamentous phage, particles. A utility of phage display lies in the fact that large libraries of randomized protein variants can be rapidly and efficiently selected for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. See e.g., Wells and Lowman, Curr. Opin. Struct. Biol, 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that selection is on the basis of intrinsic ligand affinity, and phagemid vectors are used, which simplify DNA manipulations. See e.g., Lowman and Wells, Methods: A companion to Methods in Enzymology, 3:205-0216 (1991).

In some embodiments, the panning comprises using varying binding times and concentrations to identify FLT3 binding molecules with increased or decreased on-rates, from pre-candidate FLT3 binding molecules. In some embodiments, the panning comprises using varying wash times to identify FLT3 binding molecules with increased or decreased off-rates, from pre-candidate FLT3 molecules. In some embodiments, the panning comprises using both varying binding times and varying wash times. In some embodiments, one or more stabilizing mutations are combined to increase the stability of the affinity matured FLT3 binding molecule, for example, by shuffling to create a second-stage combinatorial library from such mutants and conducting a second round of panning followed by a binding selection.

In some embodiments, the affinity matured FLT3 binding molecule comprises an equivalent or better affinity to a FLT3 protein (such as human FLT3 protein) as that of a FLT3 binding parental molecule, but that has reduced cross reactivity, or in some embodiments, increased cross reactivity, with selected substances, such as ligands, proteins, antigens, or the like, other than the FLT3 epitope for which the FLT3 binding parental molecule is specific, or is designed to be specific for. In regard to the latter, an affinity matured FLT3 binding molecule, in some embodiments, is more successfully tested in animal models if the affinity matured FLT3 binding molecule is reacted with both human FLT3 and the corresponding target of the animal model, e.g. mouse FLT3 or cynomolgus FLT3.

In some embodiments, the FLT3 binding domain is an anti-FLT3 antibody or an antigen binding fragment thereof, or a variant of the anti-FLT3 or an antigen binding fragment thereof. As used herein, the term “variant” refers to variants and derivatives of an antibody or an antigen binding fragment thereof, as described herein. In certain embodiments, amino acid sequence variants of the anti-FLT3 antibodies or antigen binding fragments thereof described herein are contemplated. For example, in certain embodiments amino acid sequence variants of anti-FLT3 antibodies or antigen binding fragments thereof described herein are contemplated to improve the binding affinity and/or other biological properties of the same. Exemplary method for preparing amino acid variants include, but are not limited to, introducing appropriate modifications into the nucleotide sequence encoding the antibody or antigen binding fragments thereof, or by peptide synthesis. Such modifications include, for example, deletions from, and/or insertions into and/or substitutions of residues within the amino acid sequences of the antibody or antigen binding fragments thereof.

Any combination of deletion, insertion, and substitution can be made to arrive at the final construct, provided that the final construct possesses the desired characteristics, e.g., antigen-binding. In certain embodiments, variants having one or more amino acid substitutions are provided. Sites of interest for substitution mutagenesis include the CDRs and framework regions. Examples of such substitutions are described below. Amino acid substitutions may be introduced into an antibody or antigen binding fragments thereof of interest and the products screened for a desired activity, e.g., retained/improved antigen binding, decreased immunogenicity, altered Antibody dependent cellular cytotoxicity (ADCC), or improved T-cell mediated cytotoxicity (TDCC). Both conservative and non-conservative amino acid substitutions are contemplated for preparing the variants.

In another example of a substitution to create a variant anti-FLT3 antibody or antigen binding fragments thereof, one or more hypervariable region residues of a parent antibody or antigen binding fragments thereof are substituted. In general, variants are then selected based on improvements in desired properties compared to a parent antibody, for example, increased affinity, reduced affinity, reduced immunogenicity, increased pH dependence of binding.

In some embodiments, the FLT3 binding domain is a single domain antibody (sdAb), such as a heavy chain variable domain (VH), a variable domain (VHH) of a llama derived sdAb, a peptide, a ligand or a small molecule entity specific for FLT3. In some embodiments, the FLT3 binding domain described herein is any domain that binds to FLT3 including but not limited to domains from a monoclonal antibody, a polyclonal antibody, a recombinant antibody, a human antibody, a humanized antibody. In certain embodiments, the FLT3 binding domain is a single-domain antibody. In other embodiments, the FLT3 binding domain is a peptide. In further embodiments, the FLT3 binding domain is a small molecule.

In one embodiment, a single domain antibody corresponds to the VHH domains of naturally occurring heavy chain antibodies directed against FLT3. As further described herein, such VHH sequences can generally be generated or obtained by suitably immunizing a species of Llama with FLT3, (i.e., so as to raise an immune response and/or heavy chain antibodies directed against FLT3), by obtaining a suitable biological sample from said Llama (such as a blood sample, serum sample or sample of B-cells), and by generating VHH sequences directed against FLT3, starting from said sample, using any suitable technique.

In another embodiment, such naturally occurring VHH domains against FLT3, are obtained from naïve libraries of Camelid VHH sequences, for example by screening such a library using FLT3, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against FLT3, involves suitably immunizing a transgenic mammal that is capable of expressing heavy chain antibodies (i.e., so as to raise an immune response and/or heavy chain antibodies directed against FLT3), obtaining a suitable biological sample from said transgenic mammal (such as a blood sample, serum sample or sample of B-cells), and then generating VHH sequences directed against FLT3, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-FLT3 single domain antibody of this disclosure comprises a single domain antibody with an amino acid sequence that corresponds to the amino acid sequence of a non-human antibody and/or a naturally occurring VHH domain, e.g., a llama anti-FLT3 antibody, but that has been “humanized,” i.e., by replacing one or more amino acid residues in the amino acid sequence of said non-human anti-FLT3 and/or the naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining the anti-FLT3 single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-FLT3 single domain antibody of the disclosure or a nucleotide sequence or nucleic acid encoding the same.

In some embodiments, the FLT3 binding domain is an anti-FLT3 specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the FLT3 binding domain comprises any domain that binds to FLT3 including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the FLT3 binding domain is a single domain antibody. In some embodiments, the anti-FLT3 single domain antibody comprises heavy chain variable complementarity determining regions (CDR), CDR1, CDR2, and CDR3.

In some embodiments, the FLT3 binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the FLT3 binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues.

In some embodiments, the binding proteins described herein comprise a polypeptide having a sequence selected from the group consisting of SEQ ID NOS: 1004-1079, subsequences thereof, and variants thereof. In some embodiments, the FLT3 binding protein comprises at least 60%-95% or more homology to a sequence selected from the group consisting of SEQ ID NOS: 1004-1079, subsequences thereof, and variants thereof. In some embodiments, the FLT3 binding protein comprises at least 60%, 61%, 62%, 63%, 63%, 65%, 66%, 67%, 68%, 69%, 70%, 75%, 80%, 85%, 90%, 95%, 96%, 97%, 98%, 99%, or more homology to a sequence selected from the group consisting of SEQ ID NOS: 1004-1079, subsequences thereof, and variants thereof. In some embodiments, the FLT3 binding protein comprises at least 60%-95% or more identity to a sequence selected from the group consisting of SEQ ID NOS: 1004-1079, subsequences thereof, and variants thereof. In some embodiments, the FLT3 binding protein comprises at least 60%, 61%, 62%, 63%, 63%, 65%, 66%, 67%, 68%, 69%, 70%, 75%, 80%, 85%, 90%, 95%, 96%, 97%, 98%, 99%, or more identity to a sequence selected from the group consisting of SEQ ID NOS: 1004-1079, subsequences thereof, and variants thereof.

In some embodiments, the CDR1 comprises the amino acid sequence selected from the group consisting of SEQ ID NOS: 1080-1155 and 3497-3498, or a sequence comprising one or more amino acid substitutions in a sequence selected from the group consisting of SEQ ID NOS: 1080-1155 and 3497-3498. In some embodiments, the CDR2 comprises the amino acid sequence selected from the group consisting of SEQ ID NOS: 1156-1231 and 3499-3500, or a sequence comprising one or more amino acid substitutions in a sequence selected from the group consisting of SEQ ID NOS: 1156-1231 and 3499-3500. In some embodiments, the CDR3 comprises the amino acid sequence selected from the group consisting of SEQ ID NOS: 1232-1307 and 3501-3502, or a sequence comprising one or more amino acid substitutions in a sequence selected from the group consisting of SEQ ID NOS: 1232-1307, and 3501-3502. In some embodiments, the CDR1 comprises the amino acid sequence selected from the group consisting of SEQ ID NOS: 1150, 1152, 3497, and 3498, or a sequence comprising one or more amino acid substitutions in a sequence selected from the group consisting of SEQ ID NOS: 1150, 1152, 3497, and 3498. In some embodiments, the CDR2 comprises the amino acid sequence selected from the group consisting of SEQ ID NOS: 1226, 1228, 3499, and 3500, or a sequence comprising one or more amino acid substitutions in a sequence selected from the group consisting of SEQ ID NOS: 1226, 1228, 3499, and 3500. In some embodiments, the CDR3 comprises the amino acid sequence selected from the group consisting of SEQ ID NOS: 1302, 1304, 3501, and 3502 or a sequence comprising one or more amino acid substitutions in a sequence selected from the group consisting of SEQ ID NOS: 1293 or 1302.

In various embodiments, the FLT3 binding domain of the present disclosure is at least about 60%, about 61%, at least about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 1004-1079 and 3495-3496.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 1080-1155 and 3497-3498.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 1156-1231 and 3499-3500.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in SEQ ID NOS: 1232-1307 and 3501-3502.

In various embodiments, a complementarity determining region of the FLT3 binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 1074, 1076, 3495, and 3496 and wherein the FLT3 binding domain comprises a humanized FLT3 binding domain.

In some embodiments, the FLT3 binding domain is cross-reactive with human cynomolgus (cyno) and mouse FLT3. In some embodiments, the FLT3 binding domain is specific for human FLT3. In certain embodiments, the FLT3 binding domains disclosed herein bind to human FLT3 with a human Kd (hKd). In certain embodiments, the FLT3 binding domains disclosed herein bind to cynomolgus FLT3 with a cyno Kd (cKd). In certain embodiments, the FLT3 binding domains disclosed herein bind to cynomolgus FLT3 with a mouse Kd (mKd). In certain embodiments, the FLT3 binding domains disclosed herein bind to both cynomolgus FLT3 and a human FLT3, with a cyno Kd (cKd) and a human Kd (hKd), respectively. In certain embodiments, the FLT3 binding domains disclosed herein bind to cynomolgus FLT3, mouse FLT3, and a human FLT3, with a cyno Kd (cKd), mouse Kd (mKd), and a human Kd (hKd), respectively. In some embodiments, the FLT3 binding protein binds to human, mouse and cynomolgus FLT3 with comparable binding affinities (i.e., hKd, mKd and cKd values do not differ by more than ±10%). In some embodiments, the hKd, mKd and the cKd range from about 0.1 nM to about 500 nM. In some embodiments, the hKd, mKd and the cKd range from about 0.1 nM to about 450 nM. In some embodiments, the hKd, mKd and the cKd range from about 0.1 nM to about 400 nM. In some embodiments, the hKd, mKd and the cKd range from about 0.1 nM to about 350 nM. In some embodiments, the hKd, mKd and the cKd range from about 0.1 nM to about 300 nM. In some embodiments, the hKd, mKd and the cKd range from about 0.1 nM to about 250 nM. In some embodiments, the hKd, mKd and the cKd range from about 0.1 nM to about 200 nM. In some embodiments, the hKd, mKd and the cKd range from about 0.1 nM to about 150 nM. In some embodiments, the hKd, mKd and the cKd range from about 0.1 nM to about 100 nM. In some embodiments, the hKd, mKd and the cKd range from about 0.1 nM to about 90 nM. In some embodiments, the hKd, mKd and the cKd range from about 0.2 nM to about 80 nM. In some embodiments, the hKd, mKd and the cKd range from about 0.3 nM to about 70 nM. In some embodiments, the hKd, mKd and the cKd range from about 0.4 nM to about 50 nM. In some embodiments, the hKd, mKd and the cKd range from about 0.5 nM to about 30 nM. In some embodiments, the hKd, mKd and the cKd range from about 0.6 nM to about 10 nM. In some embodiments, the hKd, mKd and the cKd range from about 0.7 nM to about 8 nM. In some embodiments, the hKd, mKd and the cKd range from about 0.8 nM to about 6 nM. In some embodiments, the hKd, mKd and the cKd range from about 0.9 nM to about 4 nM. In some embodiments, the hKd, mKd and the cKd range from about 1 nM to about 2 nM.

In some embodiments, any of the foregoing FLT3 binding domains (e.g., anti-FLT3 single domain antibodies of SEQ ID NOS: 1004-1079) are affinity peptide tagged for ease of purification. In some embodiments, the affinity peptide tag is six consecutive histidine residues, also referred to as 6X-His (SEQ ID NO: 3503). In certain embodiments, the FLT3 binding domains of the present disclosure preferentially bind membrane bound FLT3 over soluble FLT3 Membrane bound FLT3 refers to the presence of FLT3 in or on the cell membrane surface of a cell that expresses FLT3. Soluble FLT3 refers to FLT3 that is no longer on in or on the cell membrane surface of a cell that expresses or expressed FLT3. In certain instances, the soluble FLT3 is present in the blood and/or lymphatic circulation in a subject. In one embodiment, the FLT3 binding domains bind membrane-bound FLT3 at least 5 fold, 10 fold, 15 fold, 20 fold, 25 fold, 30 fold, 40 fold, 50 fold, 100 fold, 500 fold, or 1000 fold greater than soluble FLT3. In one embodiment, the FLT3 binding proteins of the present disclosure preferentially bind membrane-bound FLT3 30 fold greater than soluble FLT3. Determining the preferential binding of an antigen binding protein to membrane bound FLT3 over soluble FLT3 can be readily determined using binding assays.

It is contemplated that in some embodiments the FLT3 binding protein is fairly small and no more than 25 kDa, no more than 20 kDa, no more than 15 kDa, or no more than 10 kDa in some embodiments. In certain instances, the FLT3 binding protein is 5 kDa or less if it is a peptide or small molecule entity.

In other embodiments, the FLT3 binding proteins described herein comprise small molecule entity (SME) binders for FLT3. SME binders are small molecules averaging about 500 to 2000 Da in size and are attached to the FLT3 binding proteins by known methods, such as sortase ligation or conjugation. In these instances, the FLT3 binding protein comprises a domain comprising a sortase recognition sequence, e.g., LPETG (SEQ ID NO: 3200). To attach a SME binder to FLT3 binding protein comprising a sortase recognition sequence, the protein is incubated with a sortase and a SME binder whereby the sortase attaches the SME binder to the recognition sequence. In yet other embodiments, the FLT3 binding proteins described herein comprise a knottin peptide for binding FLT3. Knottins are disulfide-stabilized peptides with a cysteine knot scaffold and have average sizes about 3.5 kDa. Knottins have been contemplated for binding to certain tumor molecules such as FLT3. In further embodiments, the FLT3 binding proteins described herein comprise a natural FLT3 ligand.

In some embodiments, the FLT3 binding protein comprises more than one domain and are of a single-polypeptide design with flexible linkage of the domains. This allows for facile production and manufacturing of the FLT3 binding proteins as they can be encoded by single cDNA molecule to be easily incorporated into a vector. Further, in some embodiments where the FLT3 binding proteins described herein are a monomeric single polypeptide chain, there are no chain pairing issues or a requirement for dimerization. It is contemplated that, in such embodiments, the FLT3 binding proteins described herein have a reduced tendency to aggregate.

In the FLT3 binding proteins comprising more than one domain, the domains are linked by one or more internal linker. In certain embodiments, the internal linkers are “short,” i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, the internal linkers are “long,” i.e., consist of 15, 20 or 25 amino acid residues. In some embodiments, the internal linkers consist of about 3 to about 15, for example 8, 9 or 10 contiguous amino acid residues. Regarding the amino acid composition of the internal linkers, peptides are selected with properties that confer flexibility to the FLT3 binding proteins, do not interfere with the binding domains as well as resist cleavage from proteases. For example, glycine and serine residues generally provide protease resistance. Examples of internal linkers suitable for linking the domains in the FLT3 binding proteins include but are not limited to (GS)ₙ (SEQ ID NO: 3525), (GGS)ₙ (SEQ ID NO: 3526), (GGGS)ₙ (SEQ ID NO: 3527), (GGSG)ₙ (SEQ ID NO: 3528), (GGSGG)ₙ (SEQ ID NO: 3194), (GGGGS)ₙ (SEQ ID NO: 3195), (GGGGG)ₙ (SEQ ID NO: 3196), or (GGG)ₙ (SEQ ID NO: 3197), wherein n is 1, 2, 3, 4, 5, 6, 7, 8, 9, or 10. In one embodiment, the linker is (GGGGSGGGGSGGGGSGGGGS) (SEQ ID NO: 3198), (GGGGSGGGGSGGGGS) (SEQ ID NO: 3199), or (GGGGSGGGS) (SEQ ID NO: 3504).

In some cases, where the FLT3 binding protein comprises more than one domain, the domains within the FLT3 binding proteins are conjugated using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See e.g., Pavel Strp, Veracity of microbial transglutaminase, Bioconjugate Chem. 25, 5, 855-862.

In some examples are provided FLT3 binding proteins comprising more than one domain, wherein one of the domains comprises an acceptor glutamine in a constant region, which can then be conjugated to another domain via a lysine-based linker (e.g., any primary amine chain which is a substrate for TGase, e.g. comprising an alkylamine, oxoamine) wherein the conjugation occurs exclusively on one or more acceptor glutamine residues present in the targeting moiety outside of the antigen combining site (e.g., outside a variable region, in a constant region). Conjugation thus does not occur on a glutamine, e.g. an at least partly surface exposed glutamine, within the variable region. The FLT3 binding protein, in some examples, is formed by reacting one of the domains with a lysine-based linker in the presence of a TGase.

In some embodiments, where one or more domains within the FLT3 binding proteins are directly joined, a hybrid vector is made where the DNA encoding the directly joined domains are themselves directly ligated to each other. In some embodiments, where linkers are used, a hybrid vector is made where the DNA encoding one domain is ligated to the DNA encoding one end of a linker moiety and the DNA encoding another domain is ligated to the other end of the linker moiety.

In some embodiments, the FLT3 binding protein is a single chain variable fragments (scFv), single-domain antibody such as a heavy chain variable domain (VH), a light chain variable domain (VL) and a variable domain (VHH) of camelid derived single domain antibody. In other embodiments, the FLT3 binding protein is a non-Ig binding domain, i.e., an antibody mimetic, such as anticalins, affilins, affibody molecules, affimers, affitins, alphabodies, avimers, DARPins, fynomers, kunitz domain peptides, and monobodies. In further embodiments, the FLT3 binding protein is a ligand or peptide that binds to or associates with FLT3. In yet further embodiments, the FLT3 binding protein is a knottin. In yet further embodiments, the binding domain to FLT3 is a small molecular entity.

In certain embodiments, the FLT3 binding proteins according to the present disclosure may be incorporated into immune cell engaging proteins. In some embodiments, the immune cell engaging proteins comprise a CD3 binding domain, a half-life extension domain, and an FLT3 binding domain according to this disclosure. In some embodiments, the FLT3 binding trispecific protein comprises a trispecific antibody.

### EpCAM Binding Proteins

Described herein are immune cell engaging proteins that bind EpCAM, pharmaceutical compositions thereof, as well as nucleic acids, recombinant expression vectors and host cells for making such proteins thereof. Also provided are methods of using the disclosed EpCAM binding proteins in the prevention, and/or treatment of diseases, conditions and disorders. In some embodiments, the EpCAM binding proteins are part of immune cell engaging proteins that comprise an EpCAM binding domain as described herein.

The epithelial cell adhesion molecule (EpCAM) is a membrane glycoprotein that is expressed in most normal human epithelia and overexpressed in most carcinomas. This molecule is responsible for cell-to-cell adhesion and additionally participates in signaling, cell migration, proliferation and differentiation. Therefore, EpCAM has been the target of immunotherapy in clinical trials of several solid tumors. It has been found to play an important role in the detection and isolation of circulating tumor cells (CTCs). EpCAM has been shown in various studies to be beneficial in diagnosis and therapy of various carcinomas. Furthermore, in many cases, tumor cells were observed to express EpCAM to a much higher degree than their parental epithelium or less aggressive forms of said cancers. For example, EpCAM expression was shown to be significantly higher on neoplastic tissue and in adenocarcinoma than on normal prostate epithelium (n=76; p<0.0001), suggesting that increased EpCAM expression represents an early event in the development of prostate cancer. See Poczatek, J Urol., 1999, 162, 1462-1644. In addition, in the majority of both squamous and adenocarcinomas of the cervix a strong EpCAM expression has been shown to correlate with an increased proliferation and the disappearance of markers for terminal differentiation. See Litvinov, Am. J. Pathol. 1996, 148, 865-75. One example is breast cancer where overexpression of EpCAM on tumor cells is a predictor of survival. See Gastl, Lancet. 2000, 356, 1981-1982. Furthermore, EpCAM has been described as a marker for the detection of disseminated tumor cells in patients suffering from squamous cell carcinoma of the head, neck and lung. See Chaubal, Anticancer Res 1999, 19, 2237-2242, Piyathilake, Hum Pathol. 2000, 31, 482-487. Normal squamous epithelium, as found in epidermis, oral cavity, epiglottis, pharynx, larynx and esophagus did not significantly express EpCAM. See Quak, Hybridoma, 1990, 9, 377-387.

EpCAM is contemplated to serve to adhere epithelial cells in an oriented and highly ordered fashion. See Litvinov, J Cell Biol. 1997, 139, 1337-1348. Upon malignant transformation of epithelial cells the rapidly growing tumor cells are believed to abandon the high cellular order of epithelia. Consequently, the surface distribution of EpCAM is contemplated to become less restricted and the molecule better exposed on tumor cells. Due to their epithelial cell origin, tumor cells from most carcinomas are expected to express EpCAM on their surface.

EpCAM, is a 40-kDa membrane-integrated glycoprotein of 314 amino acids with specific expression in certain epithelia and on many human carcinomas. See, e.g., in Balzar, J. Mol. Med. 1999, 77, 699-712). EpCAM was discovered and subsequently cloned through its recognition by the murine monoclonal antibody 17-1A/edrecolomab. See Goettlinger, Int J Cancer. 1986; 38, 47-53 and Simon, Proc. Natl. Acad. Sci. USA. 1990; 87, 2755-2759. Monoclonal antibody 17-1A was generated by immunization of mice with human colon carcinoma cells. See Koprowski, Somatic Cell Genet. 1979, 5, 957-971. The EGF-like repeats of EpCAM were shown to mediate lateral and reciprocal interactions in homophilic cell adhesion. See, e.g., Balzar, Mol. Cell. Biol. 2001, 21, 2570-2580) and, for that reason, is predominantly located between epithelial cells (Litvinov, J Cell Biol. 1997, 139, 1337-1348, Balzar, J Mol Med. 1999, 77, 699-712 and Trebak, J Biol Chem. 2001, 276, 2299-2309).

EpCAM is also known by the following alternate names: Epithelial Cell Adhesion Molecule, Tumor-Associated Calcium Signal Transducer, Major Gastrointestinal Tumor-Associated Protein GA733-2, Adenocarcinoma-Associated Antigen, Cell Surface Glycoprotein Trop-1, Epithelial Glycoprotein 314, TACSTD1, EGP314, MIC18, TROP1, M4S1, KSA, Membrane Component Chromosome 4 Surface marker (35 kD glycoprotein), Antigen identified by monoclonal antibody AUA-1, human epithelial glycoprotein-2, epithelial cell surface antigen, epithelial glycoprotein, KS ¼Antigen, CD326 Antigen, GA722-2, HEGP314, HNPCC8, Ep-CAM, DIAR5, EGP-2, EGP40, KS ¼, MK-1, M1S2, ESA, and EGP. Exemplary protein sequences for EpCAM is provided in UniProtkB ID Nos. P16422 and B5MCA4. In some embodiments, the EpCAM binding proteins of this disclosure binds to an EpCAM sequence provided in UniProtkB ID Nos. P16422 (SEQ ID NO:478) or B5MCA4 (SEQ ID NO: 475).

In some embodiments, the EpCAM binding domain binds to an extracellular domain of the mature EpCAM protein. The human extracellular domain sequence is provided in SEQ ID NO: 3212; the cynomolgus extracellular domain sequence is provided in SEQ ID NO: 3213; and the mouse extracellular domain sequence is provided in SEQ ID NO: 3214.

In some embodiments, the EpCAM binding domain binds to a protein comprising a truncated sequence compared to SEQ ID NO: 3208. In some embodiments, the EPCAM binding domain binds to a protein comprising the sequence of SEQ ID NO: 3208. In some embodiments, the EpCAM binding domain binds to a protein comprising a truncated sequence compared to SEQ ID NO: 3209. In some embodiments, the EpCAM binding domain binds to a protein comprising the sequence of SEQ ID NO: 3209. In some embodiments, the EpCAM binding domain binds to a protein comprising a truncated sequence compared to SEQ ID NO: 3210. In some embodiments, the EpCAM binding domain binds to a protein comprising a truncated sequence compared to SEQ ID NO: 3210. In some embodiments, the EpCAM binding domain binds to a protein comprising a truncated sequence compared to SEQ ID NO: 478. In some embodiments, the EpCAM binding domain binds to a protein comprising a truncated sequence compared to SEQ ID NO: 3211. In some embodiments, the EpCAM binding domain binds to a protein comprising a truncated sequence compared to SEQ ID NO: 3212. In some embodiments, the EpCAM binding domain binds to a protein comprising a truncated sequence compared to SEQ ID NO: 3212. In some embodiments, the EpCAM binding domain binds to a protein comprising a truncated sequence compared to SEQ ID NO: 3213. In some embodiments, the EpCAM binding domain binds to a protein comprising a truncated sequence compared to SEQ ID NO: 3213. In some embodiments, the EpCAM binding domain binds to a protein comprising a truncated sequence compared to SEQ ID NO: 3214. In some embodiments, the EpCAM binding domain binds to a protein comprising a truncated sequence compared to SEQ ID NO: 3214.

In some embodiments, the EpCAM binding domains disclosed herein recognize full-length EpCAM. In certain instances, the EpCAM binding domains disclosed herein recognize an epitope within EpCAM, such as, in some cases the EpCAM binding proteins interact with one or more amino acids found within a domain of human EpCAM. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within a domain of EpCAM. Alternatively, the epitope may consist of a plurality of non-contiguous amino acids (or amino acid sequences) located within a domain of EpCAM.

In some embodiments, the EpCAM binding proteins of this disclosure binds to the full length EpCAM protein or to a fragment thereof, such as epitope containing fragments within the full length EpCAM protein, as described above. In some cases, the epitope containing fragment comprises antigenic or immunogenic fragments and derivatives thereof of the EpCAM protein. Epitope containing fragments, including antigenic or immunogenic fragments, are, in some embodiments, 12 amino acids or more, e.g., 20 amino acids or more, 50 or 100 amino acids or more. The EpCAM fragments, in some embodiments, comprises 95% or more of the length of the full protein, 90% or more, 75% or 50% or 25% or 10% or more of the length of the full protein. In some embodiments, the epitope-containing fragments of EpCAM including antigenic or immunogenic fragments are capable of eliciting a relevant immune response in a patient. Derivatives of EpCAM include, in some embodiments, variants on the sequence in which one or more (e.g., 1-20 such as 15 amino acids, or up to 20% such as up to 10% or 5% or 1% by number of amino acids based on the total length of the protein) deletions, insertions or substitutions have been made to the EpCAM sequence provided in SEQ ID NOS: 3208-3214.

In some embodiments, substitutions comprise conservative substitutions. Derivatives and variants of, in some examples, have essentially the same biological function as the protein from which they are derived. For instance, derivatives and variants of EpCAM are, in some cases, comparably antigenic or immunogenic to the protein from which they are derived, have either the ligand-binding activity, or the active receptor-complex forming ability, or preferably both, of the protein from which they are derived, and have the same tissue distribution as EpCAM.

In some embodiments, the EpCAM binding protein specifically binds EpCAM with equivalent or better affinity as that of a reference EpCAM binding protein, and the EpCAM binding protein in such embodiments comprises an affinity matured EpCAM binding molecule, and is derived from the EpCAM binding parental molecule, comprising one or more amino acid mutations (e.g., a stabilizing mutation, a destabilizing mutation) with respect to the EpCAM binding parental molecule. In some embodiments, the affinity matured EpCAM binding molecule has superior stability with respect to selected destabilizing agents, as that of a reference EpCAM binding parental molecule. In some embodiments, the affinity matured EpCAM binding molecule is identified in a process comprising panning of one or more pre-candidate EpCAM binding molecules derived from one or more EpCAM binding parental molecule, expressed in a phage display library, against an EpCAM protein, such as a human EpCAM protein. The pre-candidate EpCAM binding molecule comprises, in some embodiments, amino acid substitutions in the variable regions, CDRs, or framework residues, relative to a parental molecule.

As used herein, “Phage display” refers to a technique by which variant polypeptides are displayed as fusion proteins to at least a portion of a coat protein on the surface of phage, e.g., filamentous phage, particles. A utility of phage display lies in the fact that large libraries of randomized protein variants can be rapidly and efficiently selected for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. See e.g., Wells and Lowman, Curr. Opin. Struct. Biol, 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that selection is on the basis of intrinsic ligand affinity, and phagemid vectors are used, which simplify DNA manipulations. See e.g., Lowman and Wells, Methods: A companion to Methods in Enzymology, 3:205-0216 (1991).

In some embodiments, the panning comprises using varying binding times and concentrations to identify EpCAM binding molecules with increased or decreased on-rates, from pre-candidate EpCAM binding molecules. In some embodiments, the panning comprises using varying wash times to identify EpCAM binding molecules with increased or decreased off-rates, from pre-candidate EpCAM molecules. In some embodiments, the panning comprises using both varying binding times and varying wash times. In some embodiments, one or more stabilizing mutations are combined to increase the stability of the affinity matured EpCAM binding molecule, for example, by shuffling to create a second-stage combinatorial library from such mutants and conducting a second round of panning followed by a binding selection.

In some embodiments, the affinity matured EpCAM binding molecule comprises an equivalent or better affinity to a EpCAM protein (such as human EpCAM protein) as that of a EpCAM binding parental molecule, but that has reduced cross reactivity, or in some embodiments, increased cross reactivity, with selected substances, such as ligands, proteins, antigens, or the like, other than the EpCAM epitope for which the EpCAM binding parental molecule is specific, or is designed to be specific for. In regard to the latter, an affinity matured EpCAM binding molecule, in some embodiments, is more successfully tested in animal models if the affinity matured EpCAM binding molecule is reacted with both human EpCAM and the corresponding target of the animal model, e.g. mouse EpCAM or cynomolgus EpCAM. In some embodiments, the parental EpCAM binding molecule binds to human EpCAM with an affinity of about 500 nM or less, 400 nM or less, 300 nM or less, 200 nM or less, 100 nM or less, 50 nM or less, 10 nM or less, and to cynomolgus EpCAM with an affinity of about 500 nM or less, 400 nM or less, 300 nM or less, 200 nM or less, 100 nM or less, 50 nM or less, 15 nM or less, or 10 nM or less. In some embodiments, the affinity matured EpCAM binding molecule, identified after one round of panning, binds to human EpCAM with an affinity of about 5 nM or less, such as 1 nM or less, and to cynomolgus EpCAM with an affinity of about 7.5 nM or less, such as 1 nM or less. In some embodiments, the affinity matured EpCAM binding molecule, identified after two rounds of panning, binds to human EpCAM with an affinity of about 2.5 nM or less, and to cynomolgus EpCAM with an affinity of about 3.5 nM or less.

In some embodiments, the EpCAM binding protein comprises an antigen-specific binding domain polypeptide that specifically bind to targets, such as targets on diseased cells, or targets on other cells that support the diseased state, such as targets on stromal cells that support tumor growth or targets on immune cells that support disease-mediated immunosuppression. In some examples, the antigen-specific binding domain includes antibodies, single chain antibodies, Fabs, Fv, T-cell receptor binding domains, ligand binding domains, receptor binding domains, domain antibodies, single domain antibodies, minibodies, nanobodies, peptibodies, or various other antibody mimics (such as affimers, affitins, alphabodies, atrimers, CTLA4-based molecules, adnectins, anticalins, Kunitz domain-based proteins, avimers, knottins, fynomers, darpins, affibodies, affilins, monobodies and armadillo repeat protein-based proteins).

In some embodiments, the EpCAM binding domain is an anti-EpCAM antibody or an antigen binding fragment thereof, or an antibody variant of the EpCAM binding domain or an antigen binding fragment thereof. As used herein, the term “antibody variant” refers to variants and derivatives of an antibody or an antigen binding fragment as described herein. In certain embodiments, amino acid sequence variants of the anti-EpCAM antibodies or antigen binding fragments thereof, as described herein, are contemplated. For example, in certain embodiments amino acid sequence variants of anti-EpCAM antibodies or antigen binding fragments thereof, as described herein, are contemplated to improve the binding affinity and/or other biological properties of the same. Exemplary method for preparing amino acid variants include, but are not limited to, introducing appropriate modifications into the nucleotide sequence encoding the antibody or antigen binding fragment thereof, or by peptide synthesis. Such modifications include, for example, deletions from, and/or insertions into and/or substitutions of residues within the amino acid sequences of the antibody or antigen binding fragments thereof.

Any combination of deletion, insertion, and substitution can be made to arrive at the final construct, provided that the final construct possesses the desired characteristics, e.g., antigen-binding. In certain embodiments, variants having one or more amino acid substitutions are provided. Sites of interest for substitution mutagenesis include the CDRs and framework regions. Examples of such substitutions are described below. Amino acid substitutions may be introduced into an antibody or antigen binding fragments thereof of interest and the products screened for a desired activity, e.g., retained/improved antigen binding, decreased immunogenicity, altered Antibody dependent cellular cytotoxicity (ADCC), or improved T-cell mediated cytotoxicity (TDCC). Both conservative and non-conservative amino acid substitutions are contemplated for preparing the antibody variants.

In another example of a substitution to create a variant anti-EpCAM antibody or antigen binding fragments thereof, one or more hypervariable region residues of a parent antibody are substituted. In general, variants are then selected based on improvements in desired properties compared to a parent antibody or antigen binding fragments thereof, for example, increased affinity, reduced affinity, reduced immunogenicity, increased pH dependence of binding.

In some embodiments, the EpCAM binding domain is a single domain antibody (sdAb) such as a heavy chain variable domain (VH), a variable domain (VHH) of a llama derived sdAb, a peptide, a ligand or a small molecule entity specific for EpCAM. In some embodiments, the EpCAM binding domain described herein is any domain that binds to EpCAM including but not limited to domains from a monoclonal antibody, a polyclonal antibody, a recombinant antibody, a human antibody, a humanized antibody. In certain embodiments, the EpCAM binding domain is a single-domain antibody. In other embodiments, the EpCAM binding domain is a peptide. In further embodiments, the EpCAM binding domain is a small molecule.

In one embodiment, a single domain antibody corresponds to the VHH domains of naturally occurring heavy chain antibodies directed against EpCAM. As further described herein, such VHH sequences can generally be generated or obtained by suitably immunizing a species of Llama with EpCAM, (i.e., so as to raise an immune response and/or heavy chain antibodies directed against EpCAM), by obtaining a suitable biological sample from said Llama (such as a blood sample, serum sample or sample of B-cells), and by generating VHH sequences directed against EpCAM, starting from said sample, using any suitable technique known in the field.

In another embodiment, such naturally occurring VHH domains against EpCAM, are obtained from naïve libraries of Camelid VHH sequences, for example by screening such a library using EpCAM, or at least one part, fragment, antigenic determinant or epitope thereof using one or more screening techniques known in the field. Such libraries and techniques are for example described in WO 99/37681, WO 01/90190, WO 03/025020 and WO 03/035694. Alternatively, improved synthetic or semi-synthetic libraries derived from naïve VHH libraries are used, such as VHH libraries obtained from naïve VHH libraries by techniques such as random mutagenesis and/or CDR shuffling, as for example described in WO 00/43507.

In a further embodiment, yet another technique for obtaining VHH sequences directed against EpCAM, involves suitably immunizing a transgenic mammal that is capable of expressing heavy chain antibodies (i.e., so as to raise an immune response and/or heavy chain antibodies directed against EpCAM), obtaining a suitable biological sample from said transgenic mammal (such as a blood sample, serum sample or sample of B-cells), and then generating VHH sequences directed against EpCAM, starting from said sample, using any suitable technique known in the field. For example, for this purpose, the heavy chain antibody-expressing rats or mice and the further methods and techniques described in WO 02/085945 and in WO 04/049794 can be used.

In some embodiments, an anti-EpCAM single domain antibody of this disclosure comprises a single domain antibody with an amino acid sequence that corresponds to the amino acid sequence of a non-human antibody and/or a naturally occurring VHH domain, e.g., a llama anti-EpCAM antibody, but that has been “humanized,” i.e., by replacing one or more amino acid residues in the amino acid sequence of said non-human anti-EpCAM and/or the naturally occurring VHH sequence (and in particular in the framework sequences) by one or more of the amino acid residues that occur at the corresponding position(s) in a VH domain from a conventional 4-chain antibody from a human being (e.g., as indicated above).

Other suitable methods and techniques for obtaining the anti-EpCAM single domain antibody of the disclosure and/or nucleic acids encoding the same, starting from naturally occurring VH sequences or VHH sequences for example comprises combining one or more parts of one or more naturally occurring VH sequences (such as one or more framework (FR) sequences and/or complementarity determining region (CDR) sequences), one or more parts of one or more naturally occurring VHH sequences (such as one or more FR sequences or CDR sequences), and/or one or more synthetic or semi-synthetic sequences, in a suitable manner, so as to provide an anti-EpCAM single domain antibody of the disclosure or a nucleotide sequence or nucleic acid encoding the same.

In some embodiments, the EpCAM binding domain is an anti-EpCAM specific antibody comprising a heavy chain variable complementarity determining region CDR1, a heavy chain variable CDR2, a heavy chain variable CDR3, a light chain variable CDR1, a light chain variable CDR2, and a light chain variable CDR3. In some embodiments, the EpCAM binding domain comprises any domain that binds to EpCAM including but not limited to domains from a monoclonal antibody, a polyclonal antibody, a recombinant antibody, a human antibody, a humanized antibody, or antigen binding fragments such as single domain antibodies (sdAb), Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain. In some embodiments, the EpCAM binding domain is a single domain antibody. In some embodiments, the anti-EpCAM single domain antibody comprises heavy chain variable complementarity determining regions (CDR), CDR1, CDR2, and CDR3.

In some embodiments, the EpCAM binding domain is a polypeptide comprising an amino acid sequence that is comprised of four framework regions/sequences (f1-f4) interrupted by three complementarity determining regions/sequences, as represented by the formula: f1-r1-f2-r2-f3-r3-f4, wherein r1, r2, and r3 are complementarity determining regions CDR1, CDR2, and CDR3, respectively, and f1, f2, f3, and f4 are framework residues. The framework residues of the EpCAM binding protein of the present disclosure comprise, for example, 75, 76, 77, 78, 79, 80, 81, 82, 83, 84, 85, 86, 87, 88, 89, 90, 91, 92, 93, or 94 amino acid residues, and the complementarity determining regions comprise, for example, 24, 25, 26, 27, 28, 29, 30, 31, 32, 33, 34, 35, or 36 amino acid residues. In some embodiments, the EpCAM binding domain comprises an amino acid sequence selected from the group consisting of SEQ ID NOS: 961-1003.

In some embodiments, the EpCAM binding protein comprises at least 60%, 61%, 62%, 63%, 63%, 65%, 66%, 67%, 68%, 69%, 70%, 75%, 80%, 85%, 90%, 95%, 96%, 97%, 98%, 99%, or more homology to a sequence selected from the group consisting of SEQ ID NOS: 961-1003, subsequences thereof, and variants thereof. In some embodiments, the EpCAM binding protein comprises at least 70%-95% or more identity to a sequence selected from the group consisting of SEQ ID NOS: 961-1003, subsequences thereof, and variants thereof. In some embodiments, the EpCAM binding protein comprises at least 60%, 61%, 62%, 63%, 63%, 65%, 66%, 67%, 68%, 69%, 70%, 75%, 80%, 85%, 90%, 95%, 96%, 97%, 98%, 99% or more identity to a sequence selected from the group consisting of SEQ ID NOS: 961-1003, subsequences thereof, and variants thereof.

In some embodiments, the CDR1 comprises the amino acid sequence as set forth in any one of SEQ ID NOS: 847-884 or a sequence comprising one or more substitutions compared to a sequence selected from the group consisting of SEQ ID NOS: 847-884. In some embodiments, the CDR2 comprises a sequence as set forth in any one of SEQ ID NOS:885-922 or a sequence comprising one or more substitutions compared to a sequence selected from the group consisting of SEQ ID NOS: 885-922. In some embodiments, the CDR3 comprises a sequence as set forth in any one of SEQ ID NOS: 923-960 a sequence comprising one or more substitutions compared to a sequence selected from the group consisting of SEQ ID NOS: 923-960.

In various embodiments, the EpCAM binding domain of the present disclosure is at least about 60%, about 61%, at least about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to an amino acid sequence selected from the group consisting of SEQ ID NOS: 961-1003.

In various embodiments, a complementarity determining region of the EpCAM binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 847-884.

In various embodiments, a complementarity determining region of the EpCAM binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 885-922.

In various embodiments, a complementarity determining region of the EpCAM binding domain of the present disclosure is at least about 10%, about 20%, about 30%, about 40%, about 50%, about 60%, about 70%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identical to the amino acid sequence set forth in any one of SEQ ID NOS: 923-960.

In some embodiments, the EpCAM binding domain is cross-reactive with human cynomolgus and mouse EpCAM. In some embodiments, the EpCAM binding domain is specific for human EpCAM. In certain embodiments, the EpCAM binding domains disclosed herein bind to human EpCAM with a human Kd (hKd). In certain embodiments, the EpCAM binding domains disclosed herein bind to cynomolgus EpCAM with a cyno Kd (cKd). In certain embodiments, the EpCAM binding domains disclosed herein bind to cynomolgus EpCAM with a mouse Kd (mKd). In certain embodiments, the EpCAM binding domains disclosed herein bind to both cynomolgus EpCAM and a human EpCAM, with a cyno Kd (cKd) and a human Kd (hKd), respectively. In certain embodiments, the EpCAM binding domains disclosed herein bind to cynomolgus EpCAM, mouse EpCAM, and a human EpCAM, with a cyno Kd (cKd), mouse Kd (mKd), and a human Kd (hKd), respectively. In some embodiments, the EpCAM binding protein binds to human, mouse and cynomolgus EpCAM with comparable binding affinities (i.e., hKd, mKd and cKd values do not differ by more than ±10%). In some embodiments, the hKd, mKd and the cKd range from about 0.001 nM to about 500 nM. In some embodiments, the hKd, mKd and the cKd range from about 0.001 nM to about 450 nM. In some embodiments, the hKd, mKd and the cKd range from about 0.001 nM to about 400 nM. In some embodiments, the hKd, mKd and the cKd range from about 0.001 nM to about 350 nM. In some embodiments, the hKd, mKd and the cKd range from about 0.001 nM to about 300 nM. In some embodiments, the hKd, mKd and the cKd range from about 0.001 nM to about 250 nM. In some embodiments, the hKd, mKd and the cKd range from about 0.001 nM to about 200 nM. In some embodiments, the hKd, mKd and the cKd range from about 0.001 nM to about 150 nM. In some embodiments, the hKd, mKd and the cKd range from about 0.001 nM to about 100 nM. In some embodiments, the hKd, mKd and the cKd range from about 0.1 nM to about 90 nM. In some embodiments, the hKd, mKd and the cKd range from about 0.2 nM to about 80 nM. In some embodiments, the hKd, mKd and the cKd range from about 0.3 nM to about 70 nM. In some embodiments, the hKd, mKd and the cKd range from about 0.4 nM to about 50 nM. In some embodiments, the hKd, mKd and the cKd range from about 0.5 nM to about 30 nM. In some embodiments, the hKd, mKd and the cKd range from about 0.6 nM to about 10 nM. In some embodiments, the hKd, mKd and the cKd range from about 0.7 nM to about 8 nM. In some embodiments, the hKd, mKd and the cKd range from about 0.8 nM to about 6 nM. In some embodiments, the hKd, mKd and the cKd range from about 0.9 nM to about 4 nM. In some embodiments, the hKd, mKd and the cKd range from about 1 nM to about 2 nM.

In some embodiments, any of the foregoing EpCAM binding domains (e.g., anti-EpCAM single domain antibodies of SEQ ID NOS: 961-1003) are affinity peptide tagged for ease of purification. In some embodiments, the affinity peptide tag is six consecutive histidine residues, also referred to as 6X-His (SEQ ID NO: 3503).

In certain embodiments, the EpCAM binding domains of the present disclosure preferentially bind membrane bound EpCAM over soluble EpCAM Membrane bound EpCAM refers to the presence of EpCAM in or on the cell membrane surface of a cell that expresses EpCAM. Soluble EpCAM refers to EpCAM that is no longer on in or on the cell membrane surface of a cell that expresses or expressed EpCAM. In certain instances, the soluble EpCAM is present in the blood and/or lymphatic circulation in a subject. In one embodiment, the EpCAM binding domains bind membrane-bound EpCAM at least 5 fold, 10 fold, 15 fold, 20 fold, 25 fold, 30 fold, 40 fold, 50 fold, 100 fold, 500 fold, or 1000 fold greater than soluble EpCAM. In one embodiment, the EpCAM binding proteins of the present disclosure preferentially bind membrane-bound EpCAM 30 fold greater than soluble EpCAM. Determining the preferential binding of an antigen binding protein to membrane bound EpCAM over soluble EpCAM can be readily determined using binding assays.

It is contemplated that in some embodiments the EpCAM binding protein is fairly small and no more than 25 kDa, no more than 20 kDa, no more than 15 kDa, or no more than 10 kDa in some embodiments. In certain instances, the EpCAM binding protein is 5 kDa or less if it is a peptide or small molecule entity.

In other embodiments, the EpCAM binding proteins described herein comprise small molecule entity (SME) binders for EpCAM. SME binders are small molecules averaging about 500 to 2000 Da in size and are attached to the EpCAM binding proteins by known methods, such as sortase ligation or conjugation. In these instances, the EpCAM binding protein comprises a domain comprising a sortase recognition sequence, e.g., LPETG (SEQ ID NO: 3200). To attach a SME binder to EpCAM binding protein comprising a sortase recognition sequence, the protein is incubated with a sortase and a SME binder whereby the sortase attaches the SME binder to the recognition sequence. In yet other embodiments, the EpCAM binding proteins described herein comprise a knottin peptide for binding EpCAM. Knottins are disufide-stabilized peptides with a cysteine knot scaffold and have average sizes about 3.5 kDa. Knottins have been contemplated for binding to certain tumor molecules such as EpCAM. In further embodiments, the EPCAM binding proteins described herein comprise a natural EpCAM ligand.

In some embodiments, the EpCAM binding protein comprises more than one domain and are of a single-polypeptide design with flexible linkage of the domains. This allows for facile production and manufacturing of the EpCAM binding proteins as they can be encoded by single cDNA molecule to be easily incorporated into a vector. Further, in some embodiments where the EpCAM binding proteins described herein are a monomeric single polypeptide chain, there are no chain pairing issues or a requirement for dimerization. It is contemplated that, in such embodiments, the EpCAM binding proteins described herein have a reduced tendency to aggregate.

In the EpCAM binding proteins comprising more than one domain, the domains are linked by one or more internal linker. In certain embodiments, the internal linkers are “short,” i.e., consist of 0, 1, 2, 3, 4, 5, 6, 7, 8, 9, 10, 11 or 12 amino acid residues. Thus, in certain instances, the internal linkers consist of about 12 or less amino acid residues. In the case of 0 amino acid residues, the internal linker is a peptide bond. In certain embodiments, the internal linkers are “long,” i.e., consist of 15, 20 or 25 amino acid residues. In some embodiments, the internal linkers consist of about 3 to about 15, for example 8, 9 or 10 contiguous amino acid residues. Regarding the amino acid composition of the internal linkers, peptides are selected with properties that confer flexibility to the EpCAM binding proteins, do not interfere with the binding domains as well as resist cleavage from proteases. For example, glycine and serine residues generally provide protease resistance. Examples of internal linkers suitable for linking the domains in the EpCAM binding proteins include but are not limited to (GS)ₙ (SEQ ID NO: 3525), (GGS)ₙ (SEQ ID NO: 3526), (GGGS)ₙ (SEQ ID NO: 3527), (GGSG)ₙ (SEQ ID NO: 3528), (GGSGG)ₙ (SEQ ID NO: 3194), (GGGGS)ₙ (SEQ ID NO: 3195), (GGGGG)ₙ (SEQ ID NO: 3196), or (GGG)ₙ (SEQ ID NO: 3197), wherein n is 1, 2, 3, 4, 5, 6, 7, 8, 9, or 10. In one embodiment, the linker is (GGGGSGGGGSGGGGSGGGGS) (SEQ ID NO: 3198), (GGGGSGGGGSGGGGS) (SEQ ID NO: 3199), or (GGGGSGGGS) (SEQ ID NO: 3504).

In some cases, where the EpCAM binding protein comprises more than one domain, the domains within the EpCAM binding proteins are conjugated using an enzymatic site-specific conjugation method which involves the use of a mammalian or bacterial transglutaminase enzyme. Microbial transglutaminases (mTGs) are versatile tools in modern research and biotechnology. The availability of large quantities of relatively pure enzymes, ease of use, and lack of regulation by calcium and guanosine-5′-triphosphate (GTP) has propelled mTG to be the main cross-linking enzyme used in both the food industry and biotechnology. Currently, mTGs are used in many applications to attach proteins and peptides to small molecules, polymers, surfaces, DNA, as well as to other proteins. See, e.g., Pavel Strp, Veracity of microbial transglutaminase, Bioconjugate Chem. 25, 5, 855-862.

In some examples are provided EpCAM binding proteins comprising more than one domain, wherein one of the domains comprises an acceptor glutamine in a constant region, which can then be conjugated to another domain via a lysine-based linker (e.g., any primary amine chain which is a substrate for TGase, e.g. comprising an alkylamine, oxoamine) wherein the conjugation occurs exclusively on one or more acceptor glutamine residues present in the targeting moiety outside of the antigen combining site (e.g., outside a variable region, in a constant region). Conjugation thus does not occur on a glutamine, e.g. an at least partly surface exposed glutamine, within the variable region. The EpCAM binding protein, in some examples, is formed by reacting one of the domains with a lysine-based linker in the presence of a TGase.

In some embodiments, where one or more domains within the EpCAM binding proteins are directly joined, a hybrid vector is made where the DNA encoding the directly joined domains are themselves directly ligated to each other. In some embodiments, where linkers are used, a hybrid vector is made where the DNA encoding one domain is ligated to the DNA encoding one end of a linker moiety and the DNA encoding another domain is ligated to the other end of the linker moiety.

In some embodiments, the EpCAM binding protein is a single chain variable fragments (scFv), single-domain antibody such as a heavy chain variable domain (VH), a light chain variable domain (VL) and a variable domain (VHH) of camelid derived single domain antibody. In other embodiments, the EpCAM binding protein is a non-Ig binding domain, i.e., an antibody mimetic, such as anticalins, affilins, affibody molecules, affimers, affitins, alphabodies, avimers, DARPins, fynomers, kunitz domain peptides, and monobodies. In further embodiments, the EpCAM binding protein is a ligand or peptide that binds to or associates with EpCAM. In yet further embodiments, the EpCAM binding protein is a knottin. In yet further embodiments, the binding domain to EpCAM is a small molecular entity.

In certain embodiments, the EpCAM binding proteins according to the present disclosure may be incorporated into immune cell engaging proteins. In some embodiments, the immune cell engaging proteins comprise a CD3 binding domain, a half-life extension domain, and an EpCAM binding domain according to this disclosure. In some embodiments, the immune cell engaging protein comprises a trispecific antibody.

### CD3 Binding Domain

The immune cell engaging protein described herein comprises an immune cell engaging domain. In some embodiments, the immune cell engaging domain comprises a natural killer (NK) cell engaging domain, a T cell engaging domain, a B cell engaging domain, a dendritic cell engaging domain, a macrophage cell engaging domain, or a combination thereof. In some embodiments, the immune cell engaging protein comprises a T-cell engaging domain. In some embodiments, the T cell engaging domain is a CD3 binding domain.

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the T cell receptor (TCR) as well as and CD3ζ (zeta) altogether to comprise the T cell receptor complex. Clustering of CD3 on T cells, such as by immobilized anti-CD3 antibodies leads to T cell activation similar to the engagement of the T cell receptor but independent of its clone-typical specificity.

In one aspect, the single chain variable fragment CD3 binding proteins described herein comprise a domain which specifically binds to CD3. In one aspect, the single chain variable fragment CD3 binding proteins described herein comprise a domain which specifically binds to human CD3. In one aspect, the single chain variable fragment CD3 binding proteins described herein comprise a domain which specifically binds to cynomolgus CD3. In one aspect, the single chain variable fragment CD3 binding proteins described herein comprise a domain which binds to human CD3 and cynomolgus CD3. In some embodiments, the single chain variable fragment CD3 binding proteins described herein comprise a domain which specifically binds to CD3γ. In some embodiments, the single chain variable fragment CD3 binding proteins described herein comprise a domain which specifically binds to CD3δ. In some embodiments, the single chain variable fragment CD3 binding proteins described herein comprise a domain which specifically binds to CD38.

In another aspect is provided an immune cell engaging protein comprising a single chain variable fragment CD3 binding protein according to the present disclosure. In some embodiments, the immune cell engaging protein comprising a single chain variable fragment CD3 binding protein according to the present disclosure specifically binds to the T cell receptor (TCR). In certain instances, the immune cell engaging protein comprising a single chain variable fragment CD3 binding protein according to the present disclosure binds the α chain of the TCR. In certain instances, the immune cell engaging protein comprising a single chain variable fragment CD3 binding protein according to the present disclosure binds the β chain of the TCR.

In certain embodiments, the CD3 binding domain of the immune cell engaging protein described herein exhibit not only potent CD3 binding affinities with human CD3, but show also excellent crossreactivity with the respective cynomolgus monkey CD3 proteins. In some instances, the CD3 binding domain of the immune cell engaging protein binding proteins are cross-reactive with CD3 from cynomolgus monkey. In certain instances, the Kd for binding human CD3 (hKd) is about the same as the Kd for binding cynomolgus CD3 (cKd). In certain instances, the ratio between hKd and cKd (hKd:cKd) is between about 20:1 to about 1:2.

In some embodiments, the CD3 binding domain of immune cell engaging protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the immune cell engaging protein will ultimately be used. For example, for use in humans, it may be beneficial for the CD3 binding domain to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (CDR2), and heavy chain complementary determining region 3 (CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD3 binding domain is a single chain variable fragment (scFv) comprising a light chain and a heavy chain of an amino acid sequence provided herein. As used herein, “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some instances, scFvs which bind to CD3 are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the CD3 binding site. In certain embodiments, such scFv linkers are “short”, i.e. consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 10 or 15 contiguous amino acid residues. Regarding the amino acid composition of the scFv linkers, peptides are selected that confer flexibility, do not interfere with the variable domains as well as allow inter-chain folding to bring the two variable domains together to form a functional CD3 binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD3 binding and production yield of the scFv. Examples of peptide scFv linkers suitable for linking a variable light chain domain and a variable heavy chain domain in a scFv include but are not limited to (GS)ₙ (SEQ ID NO: 3525), (GGS)ₙ (SEQ ID NO: 3526), (GGGS)ₙ (SEQ ID NO: 3527), (GGSG)ₙ (SEQ ID NO: 3528), (GGSGG)ₙ (SEQ ID NO: 3194), (GGGGS)ₙ (SEQ ID NO: 3195), (GGGGG)ₙ (SEQ ID NO: 3196), or (GGG)ₙ (SEQ ID NO: 3197), wherein n is 1, 2, 3, 4, 5, 6, 7, 8, 9, or 10. In one embodiment, the scFv linker can be (GGGGS)₄ (SEQ ID NO: 3198) or (GGGGS)₃ (SEQ ID NO: 3199). Variation in the linker length may retain or enhance activity, giving rise to superior efficacy in activity studies.

In some embodiments, CD3 binding domain of an immune cell engaging protein has an affinity to CD3 on CD3 expressing cells with a K_{d} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of a single chain variable fragment CD3 binding protein has an affinity to CD3ε, γ, or δ with a K_{d} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, CD3 binding domain of a single chain variable fragment CD3 binding protein has low affinity to CD3, i.e., about 100 nM or greater.

In certain embodiments, the single chain variable fragment CD3 binding proteins described herein bind to human CD3 with a human Kd (hKd) and to cynomolgus CD3 with a cyno Kd (cKd). In some embodiments, hKd and cKd are between about between about 1 nM to about 2 nM, about 3 nM to about 5 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM.

In some embodiments, the hKd and cKd of the single chain variable fragment CD3 binding proteins is about the same as the Kd of a CD3 binding protein having the sequence as set forth is SEQ ID NO: 3167. In some embodiments, the hKd and cKd of the single chain variable fragment CD3 binding proteins is about 1.1 fold to about 1.5 fold the Kd of a CD3 binding protein having the sequence as set forth is SEQ ID NO: 3167. In some embodiments, the hKd and cKd of the single chain variable fragment CD3 binding proteins is about 1.5 fold to about 2 fold the Kd of a CD3 binding protein having the sequence as set forth is SEQ ID NO: 3167. In some embodiments, the hKd and cKd of the single chain variable fragment CD3 binding proteins is about 2.5 fold to about 3 fold the Kd of a CD3 binding protein having the sequence as set forth is SEQ ID NO: 3167. In some embodiments, the hKd and cKd of the single chain variable fragment CD3 binding proteins is about 3 fold to about 5 fold the Kd of a CD3 binding protein having the sequence as set forth is SEQ ID NO: 3167. In some embodiments, the hKd and cKd of the single chain variable fragment CD3 binding proteins is about 6 fold to about 15 fold the Kd of a CD3 binding protein having the sequence as set forth is SEQ ID NO: 3167. In some embodiments, the hKd and cKd of the single chain variable fragment CD3 binding proteins is about 15 fold to about 20 fold the Kd of a CD3 binding protein having the sequence as set forth is SEQ ID NO: 3167. In some embodiments, the hKd and cKd of the single chain variable fragment CD3 binding proteins is about 20 fold to about 50 fold the Kd of a CD3 binding protein having the sequence as set forth is SEQ ID NO: 3167. In some embodiments, the hKd and cKd of the single chain variable fragment CD3 binding proteins is about 55 fold to about 70 fold the Kd of a CD3 binding protein having the sequence as set forth is SEQ ID NO: 3167. In some embodiments, the hKd and cKd of the single chain variable fragment CD3 binding proteins is about 75 fold to about 100 fold the Kd of a CD3 binding protein having the sequence as set forth is SEQ ID NO: 3167. In some embodiments, the hKd and cKd of the single chain variable fragment CD3 binding proteins is about 120 fold to about 200 fold the Kd of a CD3 binding protein having the sequence as set forth is SEQ ID NO: 3167.

In some embodiments, the ratio between the hKd and cKd (hKd: cKd) ranges from about 20:1 to about 1:2. The affinity to bind to CD3 can be determined, for example, by the ability of the single chain variable fragment CD3 binding protein itself or its CD3 binding domain to bind to CD3 coated on an assay plate; displayed on a microbial cell surface; in solution; etc. The binding activity of the single chain variable fragment CD3 binding protein itself or its CD3 binding domain of the present disclosure to CD3 can be assayed by immobilizing the ligand (e.g., CD3) or the single chain variable fragment CD3 binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high or low pH, and the like and analyzed, for example, by Surface Plasmon Resonance (SPR).

In some embodiments, the single chain variable fragment CD3 binding protein has an amino acid sequence selected from the group consisting of SEQ ID NOS: 3153-3169.

In some embodiments, the single chain variable fragment CD3 binding protein has an amino acid sequence set forth as SEQ ID NO: 3153, wherein the hKd is about 3.8 nM, and wherein the cKd is about 3.5 nM. In some embodiments, the single chain variable fragment CD3 binding protein has an amino acid sequence set forth as SEQ ID NO: 3154, wherein the hKd is about 4.1 nM, and wherein the cKd is about 3.4 nM. In some embodiments, the single chain variable fragment CD3 binding protein has an amino acid sequence set forth as SEQ ID NO: 3155, wherein the hKd is about 4.3 nM, and wherein the cKd is about 4.2 nM. In some embodiments, the single chain variable fragment CD3 binding protein has an amino acid sequence set forth as SEQ ID NO: 3156, wherein the hKd is about 4.7 nM, and wherein the cKd is about 4.9 nM. In some embodiments, the single chain variable fragment CD3 binding protein has an amino acid sequence set forth as SEQ ID NO: 3157, wherein the hKd is about 6.4 nM, and wherein the cKd is about 6.6 nM. In some embodiments, the single chain variable fragment CD3 binding protein has an amino acid sequence set forth as SEQ ID NO: 3158, wherein the hKd is about 8 nM, and wherein the cKd is about 6.6 nM. In some embodiments, the single chain variable fragment CD3 binding protein has an amino acid sequence set forth as SEQ ID NO: 3159, wherein the hKd is about 20 nM, and wherein the cKd is about 17 nM. In some embodiments, the single chain variable fragment CD3 binding protein has an amino acid sequence set forth as SEQ ID NO: 3160, wherein the hKd is about 37 nM, and wherein the cKd is about 30 nM. In some embodiments, the single chain variable fragment CD3 binding protein has an amino acid sequence set forth as SEQ ID NO: 3161, wherein the hKd is about 14 nM, and wherein the cKd is about 13 nM. In some embodiments, the single chain variable fragment CD3 binding protein has an amino acid sequence set forth as SEQ ID NO: 3162, wherein the hKd is about 50 nM, and wherein the cKd is about 47 nM. In some embodiments, the single chain variable fragment CD3 binding protein has an amino acid sequence set forth as SEQ ID NO: 3163, wherein the hKd is about 16 nM, and wherein the cKd is about 16 nM. In some embodiments, the single chain variable fragment CD3 binding protein has an amino acid sequence set forth as SEQ ID NO: 3164, wherein the hKd is about 46 nM, and wherein the cKd is about 43 nM. In some embodiments, the single chain variable fragment CD3 binding protein has an amino acid sequence set forth as SEQ ID NO: 3165, wherein the hKd is about 18 nM, and wherein the cKd is about 17 nM. In some embodiments, the single chain variable fragment CD3 binding protein has an amino acid sequence set forth as SEQ ID NO: 3166, wherein the hKd is about 133 nM, and wherein the cKd is about 134 nM. In some embodiments, the single chain variable fragment CD3 binding protein has an amino acid sequence set forth as SEQ ID NO: 3168, wherein the hKd is about 117 nM, and wherein the cKd is about 115 nM. In some embodiments, the single chain variable fragment CD3 binding protein has an amino acid sequence set forth as SEQ ID NO: 3169, wherein the hKd is about 109 nM, and wherein the cKd is about 103 nM.

### Single Domain Serum Albumin Binding Protein

The immune cell engaging protein described herein is a half-life extended protein. In some embodiments, the immune cell engaging protein comprises a domain binds to serum albumin. In some embodiments, the serum albumin is human serum albumin (HSA).

Serum albumin is produced by the liver, occurs dissolved in blood plasma and is the most abundant blood protein in mammals. Albumin is essential for maintaining the oncotic pressure needed for proper distribution of body fluids between blood vessels and body tissues; without albumin, the high pressure in the blood vessels would force more fluids out into the tissues. It also acts as a plasma carrier by non-specifically binding several hydrophobic steroid hormones and as a transport protein for hemin and fatty acids. Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma. In some embodiments, the single domain serum albumin binding proteins bind to HSA. In some embodiments, the single domain serum albumin binding proteins bind to serum albumin protein from cynomolgus monkeys. In some embodiments, the single domain serum albumin binding proteins bind to HSA and serum albumin protein from cynomolgus monkeys. In some embodiments, the single domain serum albumin binding proteins also bind to mouse serum albumin protein. In some embodiments, the binding affinity towards mouse serum albumin is about 1.5-fold to about 20-fold weaker than that towards human or cynomolgus serum albumin.

Noncovalent association with albumin extends the elimination half-time of short-lived proteins. For example, a recombinant fusion of an albumin binding domain to a Fab fragment resulted in a decrease in in vivo clearance by 25-fold and 58-fold and a half-life extension of 26-fold and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action/serum half-life.

In some embodiments, the single-domain serum albumin binding proteins described herein is a single domain antibody such as a heavy chain variable domain (VH), a variable domain (VHH) of camelid derived sdAb, peptide, ligand or small molecule entity specific for serum albumin. In some embodiments, the single-domain serum albumin binding proteins described herein is a single domain antibody such as a heavy chain variable domain (VH), a variable domain (VHH) of camelid derived sdAb, peptide, ligand or small molecule entity specific for HSA. In some embodiments, the serum albumin binding domain of a single domain serum albumin binding protein described herein is any domain that binds to serum albumin including but not limited to domains from a monoclonal antibody, a polyclonal antibody, a recombinant antibody, a human antibody, a humanized antibody. In certain embodiments, the serum albumin binding domain is a single-domain antibody. In other embodiments, the serum albumin binding domain is a peptide. In further embodiments, the serum albumin binding domain is a small molecule. It is contemplated that the single domain serum albumin binding protein is fairly small and no more than 25 kD, no more than 20 kD, no more than 15 kD, or no more than 10 kD in some embodiments. In certain instances, the single domain serum albumin binding protein binding is 5 kD or less if it is a peptide or small molecule entity.

In some embodiments, the single domain serum albumin binding protein described herein is a half-life extension domain which provides for altered pharmacodynamics and pharmacokinetics of the single domain serum albumin binding protein itself. As above, the half-life extension domain extends the elimination half-time. The half-life extension domain also alters pharmacodynamic properties including alteration of tissue distribution, penetration, and diffusion of the single domain serum albumin binding protein. In some embodiments, the half-life extension domain provides for improved tissue (including tumor) targeting, tissue distribution, tissue penetration, diffusion within the tissue, and enhanced efficacy as compared with a protein without a half-life extension domain. In one embodiment, therapeutic methods effectively and efficiently utilize a reduced amount of the single domain serum albumin binding protein, resulting in reduced side effects, such as reduced non-tumor cell cytotoxicity.

Further, the binding affinity of the single domain serum albumin binding protein towards its binding target can be selected so as to target a specific elimination half-time in a particular single domain serum albumin binding protein. Thus, in some embodiments, the single domain serum albumin binding protein has a high binding affinity towards its binding target. In other embodiments, the single domain serum albumin binding protein has a medium binding affinity towards its binding target. In yet other embodiments, the single domain serum albumin binding protein has a low or marginal binding affinity towards its binding target. Exemplary binding affinities include KD of 10 nM or less (high), between 10 nM and 100 nM (medium), and greater than 100 nM (low). As above, binding affinities of the single domain serum albumin binding proteins towards binding targets are determined by known methods such as Surface Plasmon Resonance (SPR).

In certain embodiments, the single domain serum albumin binding protein disclosed herein binds to HSA with a human Kd (hKd). In certain embodiments, the single domain serum albumin binding protein disclosed herein binds to cynomolgus monkey serum albumin with a cyno Kd (cKd). In certain embodiments, the single domain serum albumin binding protein disclosed herein binds to cynomolgus monkey serum albumin with a cyno Kd (cKd) and to HSA with a human Kd (hKd). In some embodiments, the hKd ranges between 1 nM and 100 nM. In some embodiments, the hKd ranges between 1 nM and 10 nM. In some embodiments, the cKd ranges between 1 nM and 100 nM. In some embodiments, the cKd ranges between 1 nM and 10 nM. In some embodiments, the hKd and the cKd range between about 1 nM and about 5 nM or between about 5 nM and 10 nM. In some embodiments, the single domain serum albumin binding protein binds to serum albumin selected from among human serum albumin, cynomolgus serum albumin, and mouse serum albumin. In some embodiments, the single domain serum albumin binding protein binds to human serum albumin, cynomolgus serum albumin, and mouse serum albumin with comparable binding affinity (Kd). In some embodiments, the single domain serum albumin binding protein binds to human serum albumin with a human Kd (hKd) between about 1 nM and about 10 nM and to cynomolgus serum albumin with a cynomolgus Kd (cKd) between 1 nM and 10 nM. In some embodiments, the single domain serum albumin binding protein binds to mouse serum albumin with a mouse Kd (mKd) between about 10 nM and about 50 nM.

In some embodiments, the hKd is about 1.5 nM, about 1.6 nM, about 1.7 nM, about 1.8 nM, about 1.9 nM, about 2 nM, about 2.1 nM, about 2.2 nM, about 2.3 nM, about 2.4 nM, about 2.5 nM, about 2.6 nM, about 2.7 nM, about 2.8 nM, about 2.9 nM, about 3 nM, 3.1 nM, about 3.2 nM, about 3.3. nM, about 3.4 nM, about 3.5 nM, about 3.6 nM, about 3.7 nM, about 3.8 nM, about 3.9 nM, about 4 nM, about 4.5 nM, about 5 nM, about 6, about 6.5 nM, about 7 nM, about 7.5 nM, about 8 nM, about 8.5 nM, about 9.0 nM, about 9.5 nM, or about 10 nM.

In some embodiments, the cKd is about 1.5 nM, about 1.6 nM, about 1.7 nM, about 1.8 nM, about 1.9 nM, about 2 nM, about 2.1 nM, about 2.2 nM, about 2.3 nM, about 2.4 nM, about 2.5 nM, about 2.6 nM, about 2.7 nM, about 2.8 nM, about 2.9 nM, about 3 nM, 3.1 nM, about 3.2 nM, about 3.3. nM, about 3.4 nM, about 3.5 nM, about 3.6 nM, about 3.7 nM, about 3.8 nM, about 3.9 nM, about 4 nM, about 4.5 nM, about 5 nM, about 6, about 6.5 nM, about 7 nM, about 7.5 nM, about 8 nM, about 8.5 nM, about 9.0 nM, about 9.5 nM, or about 10 nM.

In some embodiments, the mKd is about 10 nM, about 11 nM, about 12 nM, about 13 nM, about 14 nM, about 15 nM, about 16 nM, about 17 nM, about 18 nM, about 19 nM, about 20 nM, about 21 nM, about 22 nM, about 23 nM, about 24 nM, about 25 nM, about 26 nM, about 27. nM, about 28 nM, about 29 nM, about 30 nM, about 31 nM, about 32 nM, about 33 nM, about 34 nM, about 35 nM, about 36 nM, about 37, about 38 nM, about 39 nM, about 40 nM, about 41 nM, about 42 nM, about 43 nM, about 44 nM, about 45 nM, about 46 nM, about 47 nM, about 48 nM, or about 50 nM.

In some embodiments, the single domain serum albumin binding protein has an amino acid sequence selected from the group consisting of SEQ ID NOS: 3185-3193.

In some embodiments, the single domain serum albumin binding protein has the amino acid sequence set forth as SEQ ID NO: 3185, and the hKd and the cKd are between about 1 nM and about 5 nM. In some embodiments, the single domain serum albumin binding protein has the amino acid sequence set forth as SEQ ID NO: 3185, and the hKd is about 2.3 nM and the cKd is about 2.4 nM. In some embodiments, the single domain serum albumin binding protein has the amino acid sequence set forth as SEQ ID NO: 3191, and the hKd and the cKd are between about 1 nM and about 5 nM. In some embodiments, the single domain serum albumin binding protein has the amino acid sequence set forth as SEQ ID NO: 3191, and the hKd is about 2.1 nM and the cKd is about 2.2 nM. In some embodiments, the single domain serum albumin binding protein has the amino acid sequence set forth as SEQ ID NO: 3186, and the hKd and the cKd are between about 1 nM and about 5 nM. In some embodiments, the single domain serum albumin binding protein has the amino acid sequence set forth as SEQ ID NO: 3186, and the hKd is about 1.9 nM and the cKd is about 1.7 nM. In some embodiments, the single domain serum albumin binding protein has the amino acid sequence set forth as SEQ ID NO: 3187, and the hKd and the cKd are between about 1 nM and about 5 nM. In some embodiments, the single domain serum albumin binding protein has the amino acid sequence set forth as SEQ ID NO: 3187, and the hKd is about 3.2 nM and the cKd is about 3.6 nM. In some embodiments, the single domain serum albumin binding protein has the amino acid sequence set forth as SEQ ID NO: 3188, and the hKd and the cKd are between about 1 nM and about 5 nM. In some embodiments, the single domain serum albumin binding protein has the amino acid sequence set forth as SEQ ID NO: 3188, and the hKd is about 2.7 nM and the cKd is about 2.6 nM. In some embodiments, the single domain serum albumin binding protein has the amino acid sequence set forth as SEQ ID NO: 3192, and the hKd and the cKd are between about 1 nM and about 5 nM. In some embodiments, the single domain serum albumin binding protein has the amino acid sequence set forth as SEQ ID NO: 3192, and the hKd is about 2.1 nM and the cKd is about 2 nM. In some embodiments, the single domain serum albumin binding protein has the amino acid sequence set forth as SEQ ID NO: 3189, and the hKd and the cKd are between about 5 nM and about 10 nM. In some embodiments, the single domain serum albumin binding protein has the amino acid sequence set forth as SEQ ID NO: 3189, and the hKd is about 6 nM and the cKd is about 7.5 nM. In some embodiments, the single domain serum albumin binding protein has the amino acid sequence set forth as SEQ ID NO: 3190, and wherein the hKd and the cKd are between about 1 nM and about 5 nM. In some embodiments, the single domain serum albumin binding protein has the amino acid sequence set forth as SEQ ID NO: 3190, and wherein the hKd is about 2.2 nM and the cKd is about 2.3 nM. In some embodiments, the single domain serum albumin binding protein has the amino acid sequence set forth as SEQ ID NO: 3193 and wherein the hKd and the cKd are between about 1 nM and about 5 nM. In some embodiments, the single domain serum albumin binding protein has the amino acid sequence set forth as SEQ ID NO: 3193 and wherein the hKd is about 1.6 nM and the cKd is about 1.6 nM.

In some embodiments, the single domain serum albumin binding protein has the amino acid sequence set forth as SEQ ID NO: 3185 and has a mKd of about 17 nM. In some embodiments, the single domain serum albumin binding protein has the amino acid sequence set forth as SEQ ID NO: 3186 and has a mKd of about 12 nM. In some embodiments, the single domain serum albumin binding protein has the amino acid sequence set forth as SEQ ID NO: 3187 and has a mKd of about 33 nM. In some embodiments, the single domain serum albumin binding protein has the amino acid sequence set forth as SEQ ID NO: 3188 and has a mKd of about 14 nM. In some embodiments, the single domain serum albumin binding protein has the amino acid sequence set forth as SEQ ID NO: 3190 and has a mKd of about 16 nM. In some embodiments, the single domain serum albumin binding protein has the amino acid sequence set forth as SEQ ID NO: 3191 and has a mKd of about 17 nM. In some embodiments, the single domain serum albumin binding protein has the amino acid sequence set forth as SEQ ID NO: 3192 and has a mKd of about 17 nM. In some embodiments, the single domain serum albumin binding protein has the amino acid sequence set forth as SEQ ID NO: 3193 and has a mKd of about 16 nM.

In some embodiments, the ratio between the hKd and cKd (hKd: cKd) ranges from about 20:1 to about 1:2.

In some embodiments, the single domain serum albumin binding protein has an elimination half-time of at least 1 hour, at least 2 hours, at least 4 hours, at least 6 hours, at least 12 hours, at least 20 hours, at least 25 hours, at least 30 hours, at least 35 hours, at least 40 hours, at least 45 hours, at least 50 hours, or at least 100 hours.

### Immune Cell Engaging Protein Modifications

The immune cell engaging protein described herein, including antigen binding domains and immune cell engaging domains encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the immune cell engaging protein described herein, including the peptide backbone, the amino acid side-chains, and the amino or carboxyl termini. Certain common peptide modifications that are useful for modification of the FLT3 binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, derivatives of the immune cell engaging protein as described herein comprise immunoreactive modulator derivatives and antigen binding molecules comprising one or more modifications.

In some embodiments, the immune cell engaging protein of the disclosure are monovalent or multivalent bivalent, trivalent, etc.). As used herein, the term “valency” refers to the number of potential target binding sites associated with an antibody. Each target binding site specifically binds one target molecule or specific position or locus on a target molecule. When an antibody is monovalent, each binding site of the molecule will specifically bind to a single antigen position or epitope. When an antibody comprises more than one target binding site (multivalent), each target binding site may specifically bind the same or different molecules (e.g., may bind to different ligands or different antigens, or different epitopes or positions on the same antigen).

In some embodiments, the immune cell engaging protein as set forth above are fused to an Fc region from any species, including but not limited to, human immunoglobulin, such as human IgG1, a human IgG2, a human IgG3, human IgG4, to generate Fc-fusion FLT3 binding proteins. In some embodiments, the Fc-fusion immune cell engaging protein of this disclosure have extended half-life compared to an otherwise identical immune cell engaging protein. In some embodiments, the Fc-fusion immune cell engaging protein of this disclosure contain inter alia one or more additional amino acid residue substitutions, mutations and/or modifications, e.g., in the Fc region. which result in a binding protein with preferred characteristics including, but not limited to: altered pharmacokinetics, extended serum half-life.

In some embodiments, such Fc-fused immune cell engaging protein provide extended half-lives in a mammal, such as in a human, of greater than 5 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-life, in some cases, results in a higher serum titer which thus reduces the frequency of the administration of the immune cell engaging protein and/or reduces the concentration of the antibodies to be administered. Binding to human FcRn in vivo and serum half-life of human FcRn high affinity binding polypeptides is assayed, in some examples, in transgenic mice or transfected human cell lines expressing human FcRn, or in primates to which the polypeptides with a variant Fc region are administered.

The immune cell engaging protein, in some cases, are differentially modified during or after production, e.g., by glycosylation, acetylation, phosphorylation, amidation, derivatization by known protecting/blocking groups, proteolytic cleavage, linkage to an antibody molecule or other cellular ligand, etc. Any of numerous chemical modifications are carried out by techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

Various post-translational modifications of the immune cell engaging protein also encompassed by the disclosure include, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. Moreover, the FLT3 binding proteins are, in some cases, modified with a detectable label, such as an enzymatic, fluorescent, radioisotopic or affinity label to allow for detection and isolation of the modulator.

### Polynucleotides Encoding the Immune Cell Engaging Protein

Also provided, in some embodiments, are polynucleotide molecules encoding immune cell engaging protein described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the immune cell engaging protein or gene encoding various domains of the immune cell engaging protein comprising more than one domain. In some embodiments, the gene encoding the domains are either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide coding for an immune cell engaging protein as described herein is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described immune cell engaging protein. Examples of expression vectors for expression in E. coli are pSKK (Le Gall et al., J Immunol Methods. (2004) 285(1):111-27) or pcDNA5 (Invitrogen) for expression in mammalian cells.

Thus, the immune cell engaging protein as described herein, in some embodiments, are produced by introducing a vector encoding the protein as described above into a host cell and culturing said host cell under conditions whereby the protein domains are expressed, may be isolated and, optionally, further purified.

### Immunomodulators

Provided herein in some embodiments is a combination comprising an immunomodulator and an immune cell engaging protein, such as a half-life extended immune cell engaging protein. An “immunomodulatory molecule,” or an “immunomodulator,” as used interchangeably herein refers to any molecule which is capable of effecting the proliferation or activation of the cells of a subject's immune system. Such molecules include, without limitation, an immunostimulatory antibody against a co-stimulatory receptor; a modulator of an immune checkpoint molecule, prostaglandin E2 (PGE2), transforming growth factor-b (TGF-b), indoleamine 2,3-dioxygenase (IDO), nitric oxide, hepatocyte growth factor (HGF), interleukin 6 (IL-6) and interleukin 10 (IL-10). In some embodiments, modulation of immune response means increase or decrease in the level of an immune cell.

In some embodiments, the immunomodulator is an antagonist of an immune checkpoint molecule. Examples of immune checkpoint molecules, include, but are not limited to programmed cell death 1 (PDCD1, PD1, PD-1), CD274 (CD274, PDL1, PD-L1), PD-L2, cytotoxic T-lymphocyte associated protein 4 (CTLA4, CD152), CD276 (B7H3); V-set domain containing T cell activation inhibitor 1 (VTCN1, B7H4), CD272 (B and T lymphocyte associated (BTLA)), killer cell immunoglobulin like receptor, three Ig domains and long cytoplasmic tail 1 (KIR, CD158E1), lymphocyte activating 3 (LAG3, CD223), hepatitis A virus cellular receptor 2 (HAVCR2, TIMD3, TIM3), V-set immunoregulatory receptor (VSIR, B7H5, VISTA), T cell immunoreceptor with Ig and ITIM domains (TIGIT), programmed cell death 1 ligand 2 (PDCD1LG2, PD-L2, CD273), immunoglobulin superfamily member 11 (IGSF11, VSIG3), TNFRSF14 (HVEM, CD270), TNFSFI4 (HVEML), PVR related immunoglobulin domain containing (PVRIG, CD112R), galectin 9 (LGALS9), killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1), killer cell lectin like receptor C1 (KLRC1, NKG2A, CD159A), killer cell lectin like receptor D1 (KLRD1, CD94), killer cell lectin like receptor G1 (KLRG1, CLEC15A, MAFA, 2F1), sialic acid binding Ig like lectin 7 (SIGLEC7), or sialic acid binding Ig like lectin 9 (SIGLEC9), CEACAM (e.g., CEACAM-1, CEACAM-3 and/or CEACAM-5), VISTA, LAIR1, CD160, 2B4, CD80, CD86, B7-H1, B7-H3 (CD276), B7-H4 (VTCN1), HVEM (TNFRSF14 or CD270), KIR, A2AR, A2BR, MHC class I, MHC class II, GAL9, adenosine, TGFR (e.g., TGFR beta), CD94/NKG2A, Siglec, IDO, TDO, CD39, CD73, GARP, CD47, PVRIG, CSF1R, and NOX. In certain embodiments, the immunomodulator is an inhibitor or antagonist of an immune checkpoint molecule (e.g., an inhibitor of PD-1, PD-L1, LAG-3, TIM-3, CEACAM (e.g., CEACAM-1, -3 and/or -5) or CTLA-4, or any combination thereof).

The term “immune checkpoint” refers to a group of molecules on the cell surface of CD4 and CD8 T cells. These molecules can effectively serve as “brakes” to down-modulate or inhibit an anti-tumor immune response. Inhibition of an inhibitory molecule can be performed by inhibition at the DNA, RNA or protein level. In some embodiments, an inhibitory nucleic acid (e.g., a dsRNA, siRNA or shRNA), is used to inhibit expression of an inhibitory molecule. In some embodiments, the inhibitor of an inhibitory signal is, a polypeptide e.g., a soluble ligand, or an antibody or antigen-binding fragment thereof, that binds to the inhibitory molecule.

PD-1

Immune checkpoint molecules useful in the methods and compositions of this disclosure, in some embodiments, includes, Programmed Death 1 (PD-1). PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein.

In some embodiments, the immunomodulator is an immune checkpoint modulator, e.g., an anti-PD-1 antibody selected from the group consisting of Pembrolizumab (humanized antibody), Pidilizumab (CT-011, monoclonal antibody, binds DLL1 and PD-1), Spartalizumab (PDR001, monoclonal antibody), Nivolumab (BMS-936558, MDX-1106, human IgG4 monoclonal antibody), MEDI0680 (AMP-514, monoclonal antibody), Cemiplimab (REGN2810, monoclonal antibody), Dostarlimab (TSR-042, monoclonal antibody), Sasanlimab (PF-06801591, monoclonal antibody), Tislelizumab (BGB-A317, monoclonal antibody), BGB-108 (antibody), Tislelizumab (BGB-A317, antibody), Camrelizumab (INCSHR1210, SHR-1210), AMP-224, Zimberelimab (AB122, GLS-010, WBP-3055, monoclonal antibody), AK-103 (HX-008, monoclonal antibody), AK-105 (anti-PD-1 antibody), CS1003 (monoclonal antibody), HLX10 (monoclonal antibody), Retifanlimab (MGA-012, anti-PD-1 monoclonal antibody), BI-754091 (antibody), Balstilimab (AGEN2034, PD-1 antibody), toripalimab (JS-001, antibody), cetrelimab (JNJ-63723283, anti-PD-1 antibody), genolimzumab (CBT-501, anti-PD-1 antibody), LZM009 (anti-PD-1 monoclonal antibody), Prolgolimab (BCD-100, anti-PD-1 monoclonal antibody), Sym021 (antibody), ABBV-181 (antibody), BAT-1306 (antibody), JTX-4014, sintilimab (IBI-308), Tebotelimab (MGD013, PD-1/LAG-3 bispecific), MGD-019 (PD-1/CTLA4 bispecific antibody), KN-046 (PD-1/CTLA4 bispecific antibody), MEDI-5752 (CTLA4/PD-1 bispecific antibody), RO7121661 (PD-1/TIM-3 bispecific antibody), XmAb20717 (PD-1/CTLA4 bispecific antibody), and AK-104 (CTLA4/PD-1 bispecific antibody).

Antibodies that bind specifically to PD-1 with high affinity have been disclosed in U.S. Pat. Nos. 8,008,449 and 8,779,105. Other anti-PD-1 antibodies have been described in, for example, U.S. Pat. Nos. 6,808,710, 7,488,802, 8,168,757 and 8,354,509, and PCT Publication No. WO 2012/145493. The antibodies disclosed in U.S. Pat. No. 8,008,449 have been demonstrated to exhibit one or more of the following characteristics: (a) binds to human PD-1 with a KD of 1×10-7 M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates Ab responses; and (j) inhibits tumor cell growth in vivo. Anti-PD-1 antibodies useful for the present combination include antibodies that bind specifically to human PD-1 and exhibit at least one of the preceding characteristics.

In some embodiments, the anti-PD-1 antibody or an antigen binding fragment thereof is Nivolumab (CAS Registry Number: 946414-94-4). Alternative names for Nivolumab include OPDIVO®; formerly designated as 5C4, MDX-1106, MDX-1106-04, ONO-4538, or BMS-936558. Nivolumab is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Nivolumab is a PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). In another embodiment, the anti-PD-1 antibody or fragment thereof cross-competes with Nivolumab. In other embodiments, the anti-PD-1 antibody or fragment thereof binds to the same epitope as Nivolumab. In certain embodiments, the anti-PD-1 antibody has the same CDRs as Nivolumab. In one embodiment, the anti-PD-1 antibody or an antigen binding fragment thereof is Nivolumab, and having a sequence disclosed herein (or a sequence at least 80%, 85%, 90%, 95% identical or higher to the sequence specified).

In some embodiments, the Nivolumab comprises a heavy chain comprising the sequence of SEQ ID NO: 3463 and a light chain comprising the sequence of SEQ ID NO: 3464.

In some embodiments, the anti-PD-1 antibody or an antigen binding fragment thereof is Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. Nos. 8,354,509 and 8,900,587 and WO2009/114335. In one embodiment, the anti-PD-1 antibody or an antigen binding fragment thereof is Pembrolizumab disclosed in, e.g., U.S. Pat. Nos. 8,354,509 and 8,900,587 and WO 2009/114335, and having a sequence disclosed herein (or a sequence at least 80%, 85%, 90%, 95% identical or higher to the sequence specified). In some embodiments, the Pembrolizumab comprises a heavy chain comprising the sequence of SEQ ID NO: 3465 and a light chain comprising the sequence of SEQ ID NO: 3466.

In another embodiment, the anti-PD-1 antibody or fragment thereof cross-competes with Pembrolizumab. In some embodiments, the anti-PD-1 antibody or fragment thereof binds to the same epitope as Pembrolizumab. In certain embodiments, the anti-PD-1 antibody has the same CDRs as Pembrolizumab. In another embodiment, the anti-PD-1 antibody is Pembrolizumab.

In other embodiments, the anti-PD-1 antibody or fragment thereof cross-competes with MEDI0608. In still other embodiments, the anti-PD-1 antibody or fragment thereof binds to the same epitope as MEDI0608. In certain embodiments, the anti-PD-1 antibody has the same CDRs as MEDI0608. In other embodiments, the anti-PD-1 antibody is MEDI0608 (formerly AMP-514), which is a monoclonal antibody. MEDI0608 is described, for example, in U.S. Pat. No. 8,609,089B2.

In certain embodiments, the immunomodulator is an anti-PD-1 antagonist. One example of the anti-PD-1 antagonist is AMP-224, which is a B7-DC Fc fusion protein. AMP-224 is discussed in U.S. Publ. No. 2013/0017199.

In other embodiments, the anti-PD-1 antibody or fragment thereof cross-competes with BGB-A317. In some embodiments, the anti-PD-1 antibody or fragment thereof binds the same epitope as BGB-A317. In certain embodiments, the anti-PD-1 antibody has the same CDRs as BGB-A317. In certain embodiments, the anti-PD-1 antibody is BGB-A317, which is a humanized monoclonal antibody. BGB-A317 is described in U.S. Publ. No. 2015/0079109.

Anti-PD-1 antibodies useful for the disclosed combinations also include isolated antibodies that bind specifically to human PD-1 and cross-compete for binding to human PD-1 with Nivolumab (see, e.g., U.S. Pat. Nos. 8,008,449 and 8,779,105; WO 2013/173223). The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar to those of Nivolumab by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 as, Nivolumab are mAbs. For administration to human subjects, these cross-competing antibodies can be chimeric antibodies, or humanized or human antibodies. Such chimeric, humanized or human mAbs can be prepared and isolated by methods well known in the art.

Anti-PD-1 antibodies useful for the present disclosure also include antigen-binding portions of the above antibodies. Non-limiting examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; and (iv) a Fv fragment consisting of the VL and VH domains of a single arm of an antibody.

Anti-PD-1 antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and upregulating the immune system. In certain embodiments, the anti-PD-1 antibody or antigen-binding portion thereof cross-competes with nivolumab for binding to human PD-1. In other embodiments, the anti-PD-1 antibody or antigen-binding portion thereof is a chimeric, humanized or human monoclonal antibody or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the anti-PD-1 antibody or antigen-binding portion thereof comprises a heavy chain constant region which is of a human IgG1 or IgG4 isotype. In certain other embodiments, the sequence of the IgG4 heavy chain constant region of the anti-PD-1 antibody or antigen-binding portion thereof contains an S228P mutation which replaces a serine residue in the hinge region with the proline residue normally found at the corresponding position in IgG1 isotype antibodies. This mutation, which is present in Nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the anti-PD-1 antibody or antigen-binding portion thereof is a monoclonal antibody (mAb) or an antigen-binding portion thereof. In certain embodiments of any of the therapeutic methods described herein comprising administration of an anti-PD-1 antibody, the anti-PD-1 antibody is Nivolumab. In other embodiments, the anti-PD-1 antibody is Pembrolizumab. In other embodiments, the anti-PD-1 antibody is chosen from the human antibodies 17D8, 2D3, 4H1, 4A11, 7D3 and 5F4 described in U.S. Pat. No. 8,008,449. In still other embodiments, the anti-PD-1 antibody is MEDI0608 (formerly AMP-514), AMP-224, or Pidilizumab (CT-011).

In some embodiments, the anti-PD-1 antibody or antigen binding fragment thereof is Pidilizumab. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611.

Other anti-PD-1 antibodies include, e.g., anti-PD-1 antibodies disclosed in U.S. Pat. No. 8,609,089, US 2010028330 A1, and/or US 20120114649 A1. In some embodiments, the PD-L1 inhibitor is an antibody molecule. In some embodiments, the anti-PD-L1 inhibitor is chosen from

### PD-L1 and PD-L2

“Programmed Death Ligand-1 (PD-L1)” is one of two cell surface glycoprotein ligands for PD-1 (the other being PD-L2) that down-regulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

In some embodiments, the immunomodulator is an antagonist of PD-L1, which is an anti-PD-L1 antibody. In some embodiments, the anti-PD-L1 antibody is YW243.55.570, MPDL3280A, MEDI-4736, MSB-0010718C, or MDX-1105.

In some embodiments, the immunomodulator is an immune checkpoint modulator, e.g., an anti-PD-1 antibody selected from the group consisting of Atezolizumab (MPDL3280A, monoclonal antibody; SEQ ID NOS: 3523 and 3524 provide heavy and light sequences for Atezolizumab, TECENTRIQ®, Genentech Inc.), Avelumab (MSB0010718C, monoclonal antibody), Durvalumab (MEDI-4736, human immunoglobulin G1 kappa (IgG1κ) monoclonal antibody), Envafolimab (KN035, single-domain PD-L1 antibody), AUNP12, CA-170 (small molecule targeting PD-L1 and VISTA), BMS-986189 (macrocyclic peptide), BMS-936559 (Anti-PD-L1 antibody), Cosibelimab (CK-301, monoclonal antibody), LY3300054 (antibody), CX-072 (antibody), CBT-502 (antibody), MSB-2311 (antibody), BGB-A333 (antibody), SHR-1316 (antibody), CS1001 (WBP3155, antibody), HLX-20 (antibody), KL-A167 (HBM 9167, antibody), STI-A1014 (antibody), STI-A1015 (IMC-001, antibody), BCD-135 (monoclonal antibody), FAZ-053 (antibody), CBT-502 (TQB2450, antibody), MDX1105-01 (antibody), FS-118 (LAG-3/PD-L1, bispecific antibody), M7824 (anti-PD-L1/TGF-0 receptor II fusion protein), CDX-527 (CD27/PD-L1 bispecific antibody), LY3415244 (TIM3/PD-L1 bispecific antibody), and INBRX-105 (4-1BB/PD-L1 bispecific antibody).

In some embodiments, the anti-PD-L1 antibody is MSB0010718C. MSB0010718C (also referred to as A09-246-2; Merck Serono) is a monoclonal antibody that binds to PD-L1. Additional anti-PD-L1 antibodies are disclosed in WO2013/079174, and having a sequence disclosed herein (or a sequence at least 80%, 85%, 90%, 95% identical or higher to the sequence specified). In some embodiments, the MSB0010718C comprises a heavy chain comprising the sequence of SEQ ID NO: 3467 or SEQ ID NO: 3469, and a light chain comprising the sequence of SEQ ID NO: 3468 or SEQ ID NO: 3470.

In one embodiment, the anti-PD-L1 antibody is YW243.55.570. The YW243.55.570 antibody is an anti-PD-L1 described in WO 2010/077634 (heavy and light chain variable region sequences shown in SEQ ID NOS: 3471 and 3472), and having a sequence disclosed therein (or a sequence substantially identical or similar thereto, e.g., a sequence at least 85%, 90%, 95% identical or higher to the sequence specified).

In one embodiment, the PD-L1 antibody is MDX-1105. MDX-1105, also known as BMS-936559, is an anti-PD-L1 antibody described in WO2007/005874, and having a sequence disclosed therein (or a sequence at least 80%, 85%, 90%, 95% identical or higher to the sequence specified).

In one embodiment, the PD-L1 antibody is MDPL3280A (Genentech/Roche). MDPL3280A is a human Fc optimized IgG1 monoclonal antibody that binds to PD-L1. MDPL3280A and other human monoclonal antibodies to PD-L1 are disclosed in U.S. Pat. No. 7,943,743 and U.S Publication No. 20120039906.

In some embodiments, the PD-L2 antagonist is AMP-224. AMP-224 is a PD-L2 Fc fusion soluble receptor that blocks the interaction between PD-1 and B7-H1 (B7-DCIg; Amplimmune; e.g., disclosed in WO2010/027827 and WO2011/066342).

TIM-3

The protein T cell immunoglobulin and mucin domain-3 (TIM-3) is a type I membrane protein in the immunoglobulin (Ig) superfamily. It has an extracellular Ig variable-like (IgV) domain, an extracellular mucin-like domain, and a cytoplasmic domain with six conserved tyrosine residues (Monney et al. (2002) Nature 415:536-41). TIM-3 is expressed on activated T-helper type 1 (Thl) and CD8+T (Tci) lymphocytes, some macrophages (Monney et al. (2002) Nature 415:536-41), activated natural killer (NK) cells (Ndhlovu et al. (2012) Blood 119(16):3734-43), and IL-17-producing Thi7 cells (Nakae et al. (2007) J Leukoc Biol 81: 1258-68). Studies have shown that TIM-3 functions to inhibit T cell, myeloid cell, and NK cell-mediated responses and to promote immunological tolerance. TIM-3 expression is upregulated in CD8+ T cells in cancer patients. The term TIM-3 as used herein includes human TIM-3 (hTIM-3), variants, isoforms, and species homologs of hTIM-3, and analogs having at least one common epitope with hTIM-3. The term “human TIM-3” refers to human sequence TIM-3, such as the complete amino acid sequence of human LAG-3 having UniProt Accession No. Q8TDQ0.3.

In one embodiment, a combination described herein includes a TIM-3 antibody or an antigen binding fragment thereof. In some embodiments, the combination is used to treat a cancer, e.g., a cancer described herein, e.g, a solid tumor or a hematologic malignancy. Exemplary anti-TIM-3 antibodies are disclosed in U.S. Pat. No. 8,552,156, WO 2011/155607, EP 2581113 and U.S Publication No. 2014/044728. Additional antibodies targeting TIM-3 include, but are not limited to, F38-2E2 (BioLegend), cobolimab (TSR-022; Tesaro), LY3321367 (Eli Lilly), MBG453 (Novartis) and antibodies as disclosed in, e.g., WO 2013/006490, WO 2018/085469. (e.g., antibodies comprising heavy and light chain sequences encoded by nucleic acid sequences according to SEQ ID NOS: 3510 and 3511), WO 2018/106588, WO 2018/106529 (e.g., an antibody comprising heavy and light chain sequences according to SEQ ID NOS: 3513-3514).

In another embodiment, an anti-TIM-3 antibody useful for the combination binds to the same epitope as an anti-TIM3 antibody described herein. In other embodiments, an anti-TIM-3 antibody comprises six CDRs of an anti-TIM-3 antibody as described herein.

In certain embodiments, the immunomodulator is a TIM-3 ligand inhibitor. TIM-3 ligand inhibitors include, without limitation, CEACAMI inhibitors such as the anti-CEACAMI antibody CM10 (cCAM Biotherapeutics; see WO 2013/054331), antibodies disclosed in WO 2015/075725 (e.g., CM-24, 26H7, 5F4, TEG-11, 12-140-4, 4/3/17, COL-4, F36-54, 34B1, YG-C28F2, D14HD11, M8.7.7, D11-AD11, HEA81, B 1. 1, CLB-gran-10, F34-187, T84.1, B6.2, B 1.13, YG-C94G7, 12-140-5, scFv DIATHIS1, TET-2; cCAM Biotherapeutics), antibodies described by Watt et al., 2001 (Blood, 98: 1469-1479) and in WO 2010/12557 and Phosphatidylserine inhibitors such as bavituximab (Peregrine).

### LAG-3

The term “LAG3”, “LAG-3” or “Lymphocyte Activation Gene-3” refers to Lymphocyte Activation Gene-3. The term LAG-3 as used herein includes human LAG-3 (hLAG-3), variants, isoforms, and species homologs of hLAG-3, and analogs having at least one common epitope with hLAG-3. The term “human LAG-3” refers to human sequence LAG-3, such as the complete amino acid sequence of human LAG-3 having GenBank Accession No. NP 002277. The term “mouse LAG-3” refers to mouse sequence LAG-3, such as the complete amino acid sequence of mouse LAG-3 having GenBank Accession No. NP_032505. LAG-3 is also known in the art as, for example, CD223. The human LAG-3 sequence may differ from human LAG-3 of GenBank Accession No. NP_002277 by having, e.g., conserved mutations or mutations in non-conserved regions and the LAG-3 has substantially the same biological function as the human LAG-3 of GenBank Accession No. NP_002277. For example, a biological function of human LAG-3 is having an epitope in the extracellular domain of LAG-3 that is specifically bound by an antibody of the instant disclosure or a biological function of human LAG-3 is binding to MEW Class II molecules.

In one embodiment, a combination described herein includes a LAG-3 antibody or an antigen binding fragment thereof. In some embodiments, the combination is used to treat a cancer, e.g., a cancer described herein, e.g, a solid tumor or a hematologic malignancy. In some embodiments, the anti-LAG-3 antibody is BMS-986016. BMS-986016 (also referred to as BMS986016; Bristol-Myers Squibb) is a monoclonal antibody that binds to LAG-3. BMS-986016 and other humanized anti-LAG-3 antibodies are disclosed in US 2011/0150892, WO2010/019570, and WO2014/008218. Additional anti-LAG-3 antibodies have been disclosed in Int'l Publ. No. WO/2015/042246 and U.S. Publ. Nos. 2014/0093511 and 2011/0150892. An exemplary LAG-3 antibodies useful for the present combination is 25F7 (described in U.S. Publ. No. 2011/0150892). In another embodiment, an anti-LAG-3 antibody useful for the combination binds to the same epitope as 25F7 or BMS-986016. In other embodiments, an anti-LAG-3 antibody comprises six CDRs of 25F7 or BMS-986016.

CTLA-4 Antibodies

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

In one embodiment, a combination described herein includes a CTLA-4 antibody or an antigen binding fragment thereof. In some embodiments, the combination is used to treat a cancer, e.g., a cancer described herein, e.g, a solid tumor or a hematologic malignancy.

Exemplary anti-CTLA-4 antibodies include Tremelimumab (IgG2 monoclonal antibody available from Pfizer, formerly known as ticilimumab, CP-675,206); and Ipilimumab (CTLA-4 antibody, also known as MDX-010, CAS No. 477202-00-9). Other exemplary anti-CTLA-4 antibodies are disclosed, e.g., in U.S. Pat. No. 5,811,097. In one embodiment, the CTLA4 inhibitor or antagonist of is a soluble ligand (e.g., a CTLA-4-Ig). Additional antibodies that bind specifically to CTLA-4 with high affinity have been disclosed in U.S. Pat. Nos. 6,984,720 and 7,605,238. Other anti-CTLA-4 mAbs have been described in, for example, U.S. Pat. Nos. 5,977,318, 6,051,227, 6,682,736, and 7,034,121. An exemplary clinical anti-CTLA-4 antibody is the human mAb 10D1 (now known as ipilimumab and marketed as YERVOY®) as disclosed in U.S. Pat. No. 6,984,720. Ipilimumab is an anti-CTLA-4 antibody for use in the methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma. Another anti-CTLA-4 antibody useful for the present combination is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

Anti-CTLA-4 antibodies useful for the disclosed combination also include isolated antibodies that bind specifically to human CTLA-4 and cross-compete for binding to human CTLA-4 with ipilimumab or tremelimumab or bind to the same epitope region of human CTLA-4 as ipilimumab or tremelimumab.

CD137/4-1BB

“CD137,” “CD-137,” “tumor necrosis factor receptor superfamily member 9 (TNFRSF9),” “4-1BB” and “induced by lymphocyte activation (ILA)” all refer to the same member of the tumor necrosis factor receptor family. One activity CD137 has been implicated in is costimulatory activity for activated T cells. (Jang et al. (1998) Biochem. Biophys. Res. Commun. 242 (3): 613-20). The term “CD137” as used herein includes human CD137 (h4-1BB), variants, isoforms, and species homologs of hCD137, and analogs having at least one common epitope with hCD137. The amino acid sequence for hCD137 can be found under GenBank Accession No. NP_001552.

In one embodiment, a combination described herein includes a CD137 antibody or an antigen binding fragment thereof. Anti-CD137 antibodies specifically bind to and activate CD137-expressing immune cells, stimulating an immune response, in particular a cytotoxic T cell response, against tumor cells. Antibodies that bind to CD137 have been disclosed in U.S. Publ. No. 2005/0095244 and U.S. Pat. Nos. 7,288,638, 6,887,673, 7,214,493, 6,303,121, 6,569,997, 6,905,685, 6,355,476, 6,362,325, 6,974,863, and 6,210,669. In some embodiments, the anti-CD137 antibody is urelumab (BMS-663513), described in U.S. Pat. No. 7,288,638 (20H4.9-IgG4 [1007 or BMS-663513]). In some embodiments, the anti-CD137 antibody is BMS-663031 (20H4.9-IgG1), described in U.S. Pat. No. 7,288,638. In some embodiments, the anti-CD137 antibody is 4E9 or BMS-554271, described in U.S. Pat. No. 6,887,673. In some embodiments, the anti-CD137 antibody is an antibody disclosed in U.S. Pat. Nos. 7,214,493; 6,303,121; 6,569,997; 6,905,685; or 6,355,476. In some embodiments, the anti-CD137 antibody is 1D8 or BMS-469492; 3H3 or BMS-469497; or 3E1, described in U.S. Pat. No. 6,362,325. In some embodiments, the anti-CD137 antibody is an antibody disclosed in U.S. Pat. No. 6,974,863 (such as 53A2). In some embodiments, the anti-CD137 antibody is an antibody disclosed in U.S. Pat. No. 6,210,669 (such as 1D8, 3B8, or 3E1). In some embodiments, the antibody is Pfizer's PF-05082566 (PF-2566). In other embodiments, an anti-CD137 antibody useful for the combination of this disclosure cross-competes with the anti-CD137 antibodies disclosed herein. In some embodiments, an anti-CD137 antibody binds to the same epitope as the anti-CD137 antibody disclosed herein. In other embodiments, an anti-CD137 antibody useful for the combination of this disclosure comprises six CDRs of the anti-CD137 antibodies disclosed herein.

KIR

The terms “Killer Ig-like Receptor,” “Killer Inhibitory Receptor”, or “KIR”, refers to a protein or polypeptide encoded by a gene that is a member of the KIR gene family or by a cDNA prepared from such a gene. The term KIR as used herein includes human KIR (hKIR), variants, isoforms, and species homologs of hKIR, and analogs having at least one common epitope with hKIR. The sequences of human KIR genes and cDNAs, as well as their protein products, are available in public databases, including GenBank. Non-limiting exemplary GenBank entries of human KIRs have the following accession numbers: KIR2DL1: GenBank accession number U24076, NM 014218, AAR16197, or L41267; KIR2DL2: GenBank accession number U24075 or L76669; KIR2DL3: GenBank accession number U24074 or L41268; KIR2DL4: GenBank accession number X97229; KIR2DS1: GenBank accession number X89892; KIR2DS2: GenBank accession number L76667; KIR2DS3: GenBank accession number NM_012312 or L76670 (splice variant); KIR3DL1: GenBank accession number L41269; and KIR2DS4: GenBank accession number AAR26325. A KIR may comprise from 1 to 3 extracellular domains, and may have a long (e.g., more than 40 amino acids) or short (e.g., less than 40 amino acids) cytoplasmic tail.

In one embodiment, a combination described herein includes an anti-KIR antibody or an antigen binding fragment thereof. Antibodies that bind specifically to KIR block interaction between Killer-cell immunoglobulin-like receptors (KIR) on NK cells with their ligands. Blocking these receptors facilitates activation of NK cells and, potentially, destruction of tumor cells by the latter. Examples of anti-KIR antibodies have been disclosed in Int'l Publ. Nos. WO/2014/055648, WO 2005/003168, WO 2005/009465, WO 2006/072625, WO 2006/072626, WO 2007/042573, WO 2008/084106, WO 2010/065939, WO 2012/071411 and WO/2012/160448. One anti-KIR antibody useful for the combination of this disclosure is lirilumab (also referred to as BMS-986015, IPH2102, or the S241P variant of 1-7F9), first described in Int'l Publ. No. WO 2008/084106. An additional anti-KIR antibody useful for the combination of this disclosure is 1-7F9 (also referred to as IPH2101), described in Int'l Publ. No. WO 2006/003179. In one embodiment, an anti-KIR antibody for the present combination cross competes for binding to KIR with lirilumab or I-7F9. In another embodiment, an anti-KIR antibody for the present combination binds to the same epitope as lirilumab or I-7F9. In other embodiments, an anti-KIR antibody comprises six CDRs of lirilumab or I-7F9.

GITR

GITR is a member of the tumor necrosis factor receptor super family. The term “GITR”, “tumor necrosis factor receptor superfamily member 18”, “activation-inducible TNFR family receptor” or “glucocorticoid-induced TNFR-related protein” all refer to a protein that is a member of the tumor necrosis factor receptor super family. GITR is encoded for by the TNFRSF18 gene in humans. It is a 241 amino acid type I transmembrane protein characterized by three cysteine pseudo-repeats in the extracellular domain and specifically protects T-cell receptor-induced apoptosis. Three isoforms of hGITR have been identified, all of which share the same extracellular domain, except for its C-terminal portion. Variant 1 (Accession No. NP_004186) consists of 241 amino acids and represents the longest transcript. It contains an extra coding segment that leads to a frame shift, compared to variant 2. The resulting protein (isoform 1) contains a distinct and shorter C-terminus, as compared to isoform 2. Variant 2 (Accession No. NP_683699) encodes the longest protein (isoform 2), consisting of 255 amino acids, and is soluble. Variant 3 (Accession No. NP_683700) contains an extra coding segment that leads to a frame shift, compared to variant 2. The resulting protein (isoform 3) contains a distinct and shorter C-terminus, as compared to isoform 2, and consists of 234 amino acids.

In one embodiment, a combination described herein includes an anti-GITR antibody or an antigen binding fragment thereof. Anti-GITR antibodies for combining with an anti-PD-1 antibody in a fixed dose may be any anti-GITR antibody that binds specifically to human GITR target and activate the glucocorticoid-induced tumor necrosis factor receptor (GITR). GITR is a member of the TNF receptor superfamily that is expressed on the surface of multiple types of immune cells, including regulatory T cells, effector T cells, B cells, natural killer (NK) cells, and activated dendritic cells (“anti-GITR agonist antibodies”). Specifically, GITR activation increases the proliferation and function of effector T cells, as well as abrogating the suppression induced by activated T regulatory cells. In addition, GITR stimulation promotes anti-tumor immunity by increasing the activity of other immune cells such as NK cells, antigen presenting cells, and B cells. Examples of anti-GITR antibodies have been disclosed in Int'l Publ. Nos. WO/2015/031667, WO2015/184,099, WO2015/026,684, WO11/028683 and WO/2006/105021, U.S. Pat. Nos. 7,812,135 and 8,388,967 and U.S. Publ. Nos. 2009/0136494, 2014/0220002, 2013/0183321 and 2014/0348841.

In one embodiment, an anti-GITR antibody useful for the present combination is TRX518 (described in, for example, Schaer et al. Curr Opin Immunol. (2012) April; 24(2): 217-224, and WO/2006/105021). In another embodiment, an anti-GITR antibody useful for the present combination is MK4166 or MK1248 and antibodies described in WO11/028683 and in U.S. Pat. No. 8,709,424, and comprising, e.g, a VH chain comprising SEQ ID NO: 3483 and a VL chain comprising SEQ ID NO: 3484). In certain embodiments, an anti-GITR antibody is an anti-GITR antibody that is disclosed in WO2015/031667, e.g., an antibody comprising VH CDRs 1-3 comprising SEQ ID NOS: 3485, 3486 and 3487 of WO2015/031667, respectively, and VL CDRs 1-3 comprising SEQ ID NOS: 3488, 3489 and 3490 of WO2015/031667. In certain embodiments, an anti-GITR antibody is an anti-GITR antibody that is disclosed in WO2015/184099, e.g., antibody Hum231 #1 or Hum231 #2, or the CDRs thereof, or a derivative thereof (e.g., pab1967, pab1975 or pab1979). In certain embodiments, an anti-GITR antibody is an anti-GITR antibody that is disclosed in JP2008278814, WO09/009116, WO2013/039954, US20140072566, US20140072565, US20140065152, or WO2015/026684, or is INBRX-110 (INHIBRx), LKZ-145 (Novartis), or MEDI-1873 (MedImmune). In certain embodiments, an anti-GITR antibody is an anti-GITR antibody that is described in PCT/US2015/033991 (e.g., an antibody comprising the variable regions of 28F3, 18E10 or 19D3). In some embodiments, the anti-GITR comprises a heavy chain comprising the sequence of SEQ ID NO: 3473, and a light chain comprising the sequence of SEQ ID NO: 3474. In some embodiments, the anti-GITR comprises a heavy chain comprising the sequence of SEQ ID NO: 3475, and a light chain comprising the sequence of SEQ ID NO: 3476. In some embodiments, the anti-GITR comprises a heavy chain comprising the sequence of SEQ ID NO: 3477, and a light chain comprising the sequence of SEQ ID NO: 3478. In some embodiments, the anti-GITR comprises a heavy chain comprising the sequence of SEQ ID NO: 3479, and a light chain comprising the sequence of SEQ ID NO: 3480. In some embodiments, the anti-GITR comprises a heavy chain comprising the sequence of SEQ ID NO: 3481, and a light chain comprising the sequence of SEQ ID NO: 3482.

In certain embodiments, an anti-GITR antibody for the present combination cross-competes with an anti-GITR antibody described herein, e.g., TRX518, MK4166 or an antibody comprising a VH domain and a VL domain amino acid sequence described herein. In some embodiments, an anti-GITR antibody for the present combination binds the same epitope as that of an anti-GITR antibody described herein, e.g., TRX518, MK4166 or an antibody comprising a VH domain and a VL domain amino acid sequence described herein. In certain embodiments, an anti-GITR antibody comprises the six CDRs of TRX518, MK4166 or those of an antibody comprising a VH domain and a VL domain amino acid sequence described herein.

A2AR, A2BR, CD39, CD73

In the “adenosinergic pathway” or “adenosine signaling pathway” as used herein ATP is converted to adenosine by the ectonucleotidases CD39 and CD73 resulting in inhibitory signaling through adenosine binding by one or more of the inhibitory adenosine receptors “Adenosine A2A Receptor” (A2AR, also known as ADORA2A) and “Adenosine A2B Receptor” (A2BR, also known as ADORA2B). Adenosine is a nucleoside with immunosuppressive properties and is present in high concentrations in the tumor microenvironment restricting immune cell infiltration, cytotoxicity and cytokine production. Thus, adenosine signaling is a strategy of cancer cells to avoid host immune system clearance. Adenosine signaling through A2AR and A2BR is an important checkpoint in cancer therapy that is activated by high adenosine concentrations typically present in the tumor microenvironment. CD39, CD73, A2AR and A2BR are expressed by most immune cells, including T cells, invariant natural killer cells, B cells, platelets, mast cells and eosinophils. Adenosine signaling through A2AR and A2BR counteracts T cell receptor mediated activation of immune cells and results in increased numbers of Tregs and decreased activation of DCs and effector T cells. The term “CD39” as used herein includes human CD39 (hCD39), variants, isoforms, and species homologs of hCD39, and analogs having at least one common epitope. The term “CD73” as used herein includes human CD73 (hCD73), variants, isoforms, and species homologs of hCD73, and analogs having at least one common epitope. The term “A2AR” as used herein includes human A2AR (hA2AR), variants, isoforms, and species homologs of hA2AR, and analogs having at least one common epitope. The term “A2BR” as used herein includes human A2BR (hA2BR), variants, isoforms, and species homologs of hA2BR, and analogs having at least one common epitope.

In some embodiments, the immunomodulator is an inhibitor of A2AR. A2AR inhibitors include, without limitation, small molecule inhibitors such as istradefylline (KW-6002; CAS #: 155270-99-8), PBF-509 (Palobiopharma), ciforadenant (CPI-444: Corvus Pharma/Genentech; CAS #: 1202402-40-1), ST1535 ([2butyl-9-methyl-8-(2H-1,2,3-triazol 2-yl)-9H-purin-6-xylamine]; CAS #: 496955-42-1), ST4206 (see Stasi et al., 2015, Europ J Pharm 761:353-361; CAS #: 1246018-36-9), tozadenant (SYN115; CAS #: 870070-55-6), V81444 (see WO 2002/055082), preladenant (SCH420814; Merck; CAS #: 377727-87-2), vipadenant (BUBO 14; CAS #: 442908-10-3), ST1535 (CAS #: 496955-42-1), SCH412348 (CAS #: 377727-26-9), SCH442416 (Axon 2283; Axon Medchem; CAS #: 316173-57-6), ZM241385 (4-(2-(7-amino-2-(2-furyl)-(1,2,4)triazolo(2,3-a)-(1,3,5)triazin-5-yl-amino)ethyl)phenol; Cas #: 139180-30-6), AZD4635 (AstraZeneca), AB928 (a dual A2AR/A2BR small molecule inhibitor; Arcus Biosciences) and SCH58261 (see Popoli et al., 2000, Neuropsychopharm 22:522-529; CAS #: 160098-96-4).

In some embodiments, the immunomodulator is an inhibitor of A2BR. A2BR inhibitors include, without limitation, AB928 (a dual A2AR7A2BR small molecule inhibitor; Arcus Biosciences), MRS 1706 (CAS #: 264622-53-9), GS6201 (CAS #: 752222-83-6) and PBS 1115 (CAS #: 152529-79-8). CD39 inhibitors include, without limitation, A001485 (Arcus Biosciences), PSB 069 (CAS #: 78510-31-3) and the anti-CD39 monoclonal antibody IPH5201 (Innate Pharma; see Perrot et ah, 2019, Cell Reports 8:2411-2425.E9).

In some embodiments, the immunomodulator is an inhibitor of CD39. CD39 inhibitors include, without limitation, A001485 (Arcus Biosciences), PSB 069 (CAS #: 78510-31-3) and the anti-CD39 monoclonal antibody IPH5201 (Innate Pharma; see Perrot et ah, 2019, Cell Reports 8:2411-2425.E9).

In some embodiments, the immunomodulator is an inhibitor of CD73. CD73 inhibitors include, without limitation, anti-CD73 antibodies such as CPI-006 (Corvus Pharmaceuticals), MEDI9447 (Medlmmune; see WO2016075099), IPH5301 (Innate Pharma; see Perrot et al., 2019, Cell Reports 8:2411-2425. E9), the anti-CD73 antibodies described in WO2018/110555, the small molecule inhibitors PBS 12379 (Tocris Bioscience; CAS #: 1802226-78-3), A000830, A001190 and A001421 (Arcus Biosciences; see Becker et al., 2018, Cancer Research 78(13 Supplement):3691-3691, doi: 10.1158/1538-7445.AM2018-3691), CB-708 (Calithera Biosciences) and purine cytotoxic nucleoside analogue-based diphosphonates as described by Allard et al., 2018 (Immunol Rev., 276(1): 121-144).

VISTA

“V-domain Ig suppressor of T cell activation” (VISTA, also known as C10orf54) bears homology to PD-L1 but displays a unique expression pattern restricted to the hematopoietic compartment. The term “VISTA” as used herein includes human VISTA (hVISTA), variants, isoforms, and species homologs of hVISTA, and analogs having at least one common epitope. VISTA induces T cell suppression and is expressed by leukocytes within tumors. In some embodiments, the immunomodulator is an inhibitor of VISTA. VISTA inhibitors include, without limitation, anti-VISTA antibodies such as JNJ-61610588 (onvatilimab; Janssen Biotech) and the small molecule inhibitor CA-170 (anti-PD-L1/L2 and anti-VISTA small molecule; CAS #: 1673534-76-3)

IDO

“Indoleamine 2,3-dioxygenase” (IDO) is a tryptophan catabolic enzyme with immune-inhibitory properties. The term “IDO” as used herein includes human IDO (hIDO), variants, isoforms, and species homologs of hIDO, and analogs having at least one common epitope. IDO is the rate limiting enzyme in tryptophan degradation catalyzing its conversion to kynurenine. Therefore, IDO is involved in depletion of essential amino acids. It has also been shown to be involved in suppression of T and NK cells, generation and activation of Tregs and myeloid-derived suppressor cells, and promotion of tumor angiogenesis. IDO is overexpressed in many cancers and was shown to promote immune system escape of tumor cells and to facilitate chronic tumor progression when induced by local inflammation.

In some embodiments, the immunomodulator is an inhibitor of IDO. IDO inhibitors include, without limitation, exiguamine A, epacadostat (INCB024360; InCyte; see U.S. Pat. No. 9,624,185), indoximod (Newlink Genetics; CAS #: 110117-83-4), NLG919 (Newlink Genetics/Genentech; CAS #: 1402836-58-1), GDC-0919 (Newlink Genetics/Genentech; CAS #: 1402836-58-1), F001287 (Flexus Biosciences/BMS; CAS #: 2221034-29-1), KHK2455 (Cheong et al., 2018, Expert Opin Ther Pat. 28(4):317-330), PF-06840003 (see WO 2016/181348), navoximod (RG6078, GDC-0919, NLG919; CAS #: 1402837-78-8), linrodostat (BMS-986205; Bristol-Myers Suibb; CAS #: 1923833-60-6), small molecules such as 1-methyl-tryptophan, pyrrolidine-2, 5-dione derivatives (see WO 2015/173764) and the IDO inhibitors disclosed by Sheridan, 2015, Nat Biotechnol 33:321-322.

TDO

In some embodiments the immunomodulator can target the signal mediated by “tryptophan-2, 3-dioxygenase” (TDO). TDO represents an alternative route to IDO in tryptophan degradation and is involved in immune suppression. Since tumor cells may catabolize tryptophan via TDO instead of IDO, TDO may represent an additional target for checkpoint blockade. Indeed, several cancer cell lines have been found to upregulate TDO and TDO may complement IDO inhibition. The term “TDO” as used herein includes human TDO (hTDO), variants, isoforms, and species homologs of hTDO, and analogs having at least one common epitope with hTDO. In some embodiments, the immunomodulator is an inhibitor of TDO. TDO inhibitors include, without limitation, 4-(indol-3-yl)-pyrazole derivatives (see U.S. Pat. No. 9,126,984 and US 2016/0263087), 3-indol substituted derivatives (see WO 2015/140717, WO 2017/025868, WO 2016/147144), 3-(indol-3-yl)-pyridine derivatives (see US 2015/0225367 and WO 2015/121812), dual IDO/TDO antagonist, such as small molecule dual IDO/TDO inhibitors disclosed in WO 2015/150097, WO 2015/082499, WO 2016/026772, WO 2016/071283, WO2016/071293, WO 2017/007700, and the small molecule inhibitor CB548 (Kim, C, et al., 2018, Annals Oncol 29 (suppl_8): viii400-viii441).

Siglec

The “Sialic acid binding immunoglobulin type lectin” (Siglec) family members recognize sialic acids and are involved in distinction between “self and “non-self’. The term “Siglecs” as used herein includes human Siglecs (hSiglecs), variants, isoforms, and species homologs of hSiglecs, and analogs having at least one common epitope with one or more hSiglecs. The human genome contains 14 Siglecs of which several are involved in immunosuppression, including, without limitation, Siglec-2, Siglec-3, Siglec-7 and Siglec-9. Siglec receptors bind glycans containing sialic acid, but differ in their recognition of the linkage regiochemistry and spatial distribution of sialic residues. The members of the family also have distinct expression patterns. A broad range of malignancies overexpress one or more Siglecs.

In some embodiments, the immunomodulator is an inhibitor of Siglec. Siglec inhibitors include, without limitation, the anti-Sigle-7 antibodies disclosed in US 2019/023786 and WO 2018/027203 (e.g., an antibody comprising a variable heavy chain region according to SEQ ID NO: 3515 and a variable light chain region according to SEQ ID NO: 3516), the anti-Siglec-2 antibody inotuzumab ozogamicin (Besponsa; see U.S. Pat. Nos. 8,153,768 and 9,642,918), the anti-Siglec-3 antibody gemtuzumab ozogamicin (Mylotarg; see U.S. Pat. No. 9,359,442) or the anti-Siglec-9 antibodies disclosed in US 2019/062427, US 2019/023786, WO 2019/011855, WO2019/011852, US 2017/306014 and EP 3 146 979.

CD20

“CD20” is an antigen expressed on the surface of B and T cells. High expression of CD20 can be found in cancers, such as B cell lymphomas, hairy cell leukemia, B cell chronic lymphocytic leukemia, and melanoma cancer stem cells. The term “CD20” as used herein includes human CD20 (hCD20), variants, isoforms, and species homologs of hCD20, and analogs having at least one common epitope. In some embodiments, the immunomodulator is an inhibitor of CD20. CD20 inhibitors include, without limitation, anti-CD20 antibodies such as rituximab (RITUXAN; IDEC-102; IDEC-C2B8; see U.S. Pat. No. 5,843,439), ABP 798 (rituximab biosimilar), ofatumumab (2F2; see WO2004/035607), obinutuzumab, ocrelizumab (2h7; see WO 2004/056312), ibritumomab tiuxetan (Zevalin), tositumomab, ublituximab (LFB-R603; LFB Biotechnologies) and the antibodies disclosed in US 2018/0036306 (e.g., an antibody comprising light and heavy chains according to SEQ ID NOS: 1-3 and 4-6, or 7 and 8, or 9 and 10).

GARP

“Glycoprotein A repetitions predominant” (GARP) plays a role in immune tolerance and the ability of tumors to escape the patient's immune system. The term “GARP” as used herein includes human GARP (hGARP), variants, isoforms, and species homologs of hGARP, and analogs having at least one common epitope. GARP is expressed on lymphocytes including Treg cells in peripheral blood and tumor infiltrating T cells at tumor sites. It is hypothesized to bind to latent “transforming growth factor b” (TGF-b). Disruption of GARP signaling in Tregs results in decreased tolerance and inhibits migration of Tregs to the gut and increased proliferation of cytotoxic T cells. In some embodiments, the immunomodulator is an inhibitor of GARP. GARP inhibitors include, without limitation, anti-GARP antibodies such as ARGX-115 (arGEN-X) and the antibodies and methods for their production as disclosed in US 2019/127483, US 2019/016811, US 2018/327511, US 2016/251438, EP 3 253 796.

CD47/SIRP

“CD47” is a transmembrane protein that binds to the ligand “signal-regulatory protein alpha” (SIRPa). The term “CD47” as used herein includes human CD47 (hCD47), variants, isoforms, and species homologs of hCD47, and analogs having at least one common epitope with hCD47. The term “SIRPa” as used herein includes human SIRPa (hSIRPa), variants, isoforms, and species homologs of hSIRPa, and analogs having at least one common epitope with hSIRPa. CD47 signaling is involved in a range of cellular processes including apoptosis, proliferation, adhesion and migration. CD47 is overexpressed in many cancers and functions as “don't eat me” signal to macrophages. Blocking CD47 signaling through inhibitory anti-CD47 or anti-SIRPa antibodies enables macrophage phagocytosis of cancer cells and fosters the activation of cancer-specific T lymphocytes. In some embodiments, the immunomodulator is an inhibitor of CD47. CD47 inhibitors include, without limitation, anti-CD47 antibodies such as HuF9-G4 (Stanford University/Forty Seven), CC-90002/ENBRX-103 (Celgene/Inhibrx), SRF231 (Surface Oncology), IBI188 (Innovent Biologies), AO-176 (Arch Oncology), bispecific antibodies targeting CD47 including TG-1801 (NI-1701; bispecific monoclonal antibody targeting CD47 and CD19; Novimmune/TG Therapeutics) and NI-1801 (bispecific monoclonal antibody targeting CD47 and mesothelin; Novimmune), and CD47 fusion proteins such as ALX148 (ALX Oncology; see Kauder et al., 2019, PLoS One, doi: 10.1371/joumal.pone.0201832). In some embodiments, the immunomodulator is an inhibitor of SIRPa. SIRPa inhibitors include, without limitation, anti-SIRPa antibodies such as OSE-172 (Boehringer Ingelheim/OSE), FSI-189 (Forty Seven), anti-SIRPa fusion proteins such as TTI-621 and TTI-662 (Trillium Therapeutics; see WO 2014/094122).

PVRIG

“Poliovirus receptor related immunoglobulin domain containing” (PVRIG, also known as CD112R) binds to “Poliovirus receptor-related 2” (PVRL2). PVRIG and PVRL2 are overexpressed in a number of cancers. PVRIG expression also induces TIGIT and PD-1 expression and PVRL2 and PVR (a TIGIT ligand) are co-overexpressed in several cancers. Blockade of the PVRIG signaling pathway results in increased T cell function and CD8+ T cell responses and, therefore, reduced immune suppression and elevated interferon responses. The term “PVRIG” as used herein includes human PVRIG (hPVRIG), variants, isoforms, and species homologs of hPVRIG, and analogs having at least one common epitope with hPVRIG. “PVRL2” as used herein includes hPVRL2, as defined above.

In some embodiments, the immunomodulator is an inhibitor of PVRIG. PVRIG inhibitors include, without limitation, anti-PVRIG antibodies such as COM701 (CGEN-15029) and antibodies and method for their manufacture as disclosed in, e.g., WO 2018/033798 (e.g., CHA.7.518.1H4(S241P), CHA.7.538.1.2.H4(S241P), CPA.9.086H4(S241P), CPA.9.083H4(S241P), CHA.9.547.7.H4(S241P), CHA.9.547.13.H4(S241P) and antibodies comprising a variable heavy domain according to SEQ ID NO: 3491 and a variable light domain according to SEQ ID NO: 3492 of WO 2018/033798 or antibodies comprising a heavy chain according to SEQ ID NO: 3493 and a light chain according to SEQ ID NO: 3494; WO 2018/033798 further discloses anti-TIGIT antibodies and combination therapies with anti-TIGIT and anti-PVRIG antibodies), WO2016134333, WO2018017864 and anti-PVRIG antibodies and fusion peptides as disclosed in WO 2016/134335.

CSF1R

The “colony-stimulating factor 1” pathway is another checkpoint that can be targeted according to the disclosure. CSF1R is a myeloid growth factor receptor that binds CSF1. Blockade of the CSF1R signaling can functionally reprogram macrophage responses, thereby enhancing antigen presentation and anti-tumor T cell responses. The term “CSF1R” as used herein includes human CSF1R (hCSFIR), variants, isoforms, and species homologs of hCSFIR, and analogs having at least one common epitope with hCSFIR. The term “CSF1” as used herein includes human CSF1 (hCSF1), variants, isoforms, and species homologs of hCSF1, and analogs having at least one common epitope with hCSF1.

In some embodiments, the immunomodulator is an inhibitor of CSF1R. CSF1R inhibitors include, without limitation, anti-CSFIR antibodies cabiralizumab (FPA008; FivePrime; see WO 2011/140249, WO 2013/169264 and WO 2014/036357), IMC-CS4 (EiiLilly), emactuzumab (RO5509554; Roche), RG7155 (WO 2011/70024, WO 2011/107553, WO 2011/131407, WO 2013/87699, WO 2013/119716, WO 2013/132044) and the small molecule inhibitors BLZ945 (CAS #: 953769-46-5) and pexidartinib (PLX3397; Selleckchem; CAS #: 1029044-16-3). CSF1 inhibitors include, without limitation, anti-CSF1 antibodies disclosed in EP 1 223 980 and Weir et al., 1996 (J Bone Mineral Res 11: 1474-1481), WO 2014/132072, and antisense DNA and RNA as disclosed in WO 2001/030381.

NOX

“Nicotinamide adenine dinucleotide phosphate NADPH oxidase” refers to an enzyme of the NOX family of enzymes of myeloid cells that generate immunosuppressive reactive oxygen species (ROS). Five NOX enzymes (NOX1 to NOX5) have been found to be involved in cancer development and immunosuppression. Elevated ROS levels have been detected in almost all cancers and promote many aspects of tumor development and progression. NOX produced ROS dampens NK and T cell functions and inhibition of NOX in myeloid cells improves anti-tumor functions of adjacent NK cells and T cells. The term “NOX” as used herein includes human NOX (hNOX), variants, isoforms, and species homologs of hNOX, and analogs having at least one common epitope with hNOX.

In some embodiments, the immunomodulator is an inhibitor of NOX. Exemplary NOX inhibitors include, without limitation, NOX1 inhibitors such as the small molecule ML171 (Gianni et al., 2010, ACS Chem Biol 5(10):981-93, NOS31 (Yamamoto et al., 2018, Biol Pharm Bull. 41(3):419-426), NOX2 inhibitors such as the small molecules ceplene (histamine dihydrochloride; CAS #: 56-92-8), BJ-1301 (Gautam et al., 2017, Mol Cancer Ther 16(10):2144-2156; CAS #: 1287234-48-3) and inhibitors described by Lu et al., 2017, Biochem Pharmacol 143:25-38, NOX4 inhibitors such as the small molecule inhibitors VAS2870 (Altenhofer et al., 2012, Cell Mol Life Sciences 69(14):2327-2343), diphenylene iodonium (CAS #: 244-54-2) and GKT137831 (CAS #: 1218942-37-0; see Tang et al., 2018, 19(10):578-585).

CD94/NKG2A

“CD94/NKG2A” is an inhibitory receptor predominantly expressed on the surface of natural killer cells and of CD8+ T cells. The term “CD94/NKG2A” as used herein includes human CD94/NKG2A (hCD94/NKG2A), variants, isoforms, and species homologs of hCD94/NKG2A, and analogs having at least one common epitope. The CD94/NKG2A receptor is a heterodimer comprising CD94 and NKG2A. It suppresses NK cell activation and CD8+ T cell function, probably by binding to ligands such as HLA-E. CD94/NKG2A restricts cytokine release and cytotoxic response of natural killer cells (NK cells), Natural Killer T cells (NK-T cells) and T cells (a/b and g/d). NKG2A is frequently expressed in tumor infiltrating cells and HLA-E is overexpressed in several cancers. In some embodiments, the immunomodulator is an inhibitor of CD94/NKg2A. CD94/NKG2A inhibitors include, without limitation, monalizumab (IPH2201; Innate Pharma) and the antibodies and method for their production as disclosed in U.S. Pat. No. 9,422,368 (e.g., humanized Z199; see EP 2628 753), EP 3 193 929 and WO2016/032334 (e.g., humanized Z270; see EP 2628 753). In some embodiments, an anti-NKG2A antibody has a heavy chain sequence according to any one of SEQ ID NOS: 3517-3521, and a light chain according to SEQ ID NO: 3522.

Activators

In some embodiment, the immunomodulator comprises an immune checkpoint activator, e.g., an agonist of at least one of: CD27, CD70, CD40, CD40LG, TNF receptor superfamily member 4 (TNFRSF4, OX40); TNF superfamily member 4 (TNFSF4, OX40L), GITR (TNF receptor superfamily member 18, TNFRSF18, CD357), TNFSF18 (GITRL), CD137 (TNFRSF9, tumor necrosis factor receptor superfamily member 9, 4-1BB, ILA, induced by lymphocyte activation), CD137L (TNFSF9), CD28, CD278 (inducible T cell co-stimulator, ICOS), inducible T cell co-stimulator ligand (ICOSLG, B7H2), CD80 (B7-1), nectin cell adhesion molecule 2 (NECTIN2, CD112), CD226 (DNAM-1), Poliovirus receptor (PVR) cell adhesion molecule (PVR, CD155), CD16, killer cell lectin like receptor K1 (KLRKI1, NKG2D, CD314), or SLAM family member 7 (SLAMF7). In some embodiment, the agonist is an antibody or an antigen-binding fragment thereof.

### Methods of Treatment

Provided herein in some embodiments is a method of treatment comprising administering to a subject in need thereof a combination as described herein. In some instances, a dose for the immunomodulator, e.g., an anti-PD-1 antibody molecule, e.g., pembrolizumab, is from about 1 mg/kg to about 10 mg/kg, e.g., 3 mg/kg. In one embodiment, the immunomodulator, e.g., the anti-PD-1 antibody molecule, e.g., Pembrolizumab is administered after treatment, e.g., after treatment of a cancer with the half-life extended immune cell engaging protein. In one embodiment, the immunomodulator, e.g., the anti-PD-1 antibody molecule, e.g., Pembrolizumab is administered before treatment, e.g., before treatment of a cancer with the half-life extended immune cell engaging protein. In one embodiment, the immunomodulator, e.g., the anti-PD-1 antibody molecule, e.g., Pembrolizumab is administered concurrently with the half-life extended immune cell engaging protein.

In some embodiments, a composition comprising the combination, or a composition comprising the immunomodulator, e.g., an PD-1 antibody, e.g., Pembrolizumab, is administered at a flat dose regardless of the weight of the patient. For example, an anti-PD-1 antibody in some embodiments is administered at a flat dose of about 0.1, 0.5, 1, 2, 3, 4, 5, 10, 15, 20, 50, 75, 80, 200, 240, 300, 360, 400, 480, 500, 750 or 1500 mg or any other dose disclosed herein, without regard to the patient's weight. In some embodiments, a composition comprising the combination, or a composition comprising the immunomodulator, e.g., an PD-1 antibody, e.g., Pembrolizumab, is administered at a weight-based dose at any dose disclosed herein.

In certain embodiments of the present combination therapy methods, the therapeutically effective dosage of the immunomodulator, e.g., an anti-PD-1 antibody or antigen-binding portion thereof, comprises about 60 mg, about 80 mg, about 100 mg, about 120 mg, about 140, about 160 mg, about 180 mg, about 200 mg, about 220 mg, about 240 mg, about 260 mg, about 280 mg, or about 300 mg. In some embodiments, the therapeutically effective dosage of the immunomodulator, e.g., the anti-PD-1 antibody or antigen-binding portion thereof, comprises about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg. In some embodiments, the dose of the immunomodulator, e.g., an anti-PD-1 antibody or antigen-binding portion thereof, in the composition is between about 60 mg and about 300 mg, between about 60 mg and about 100 mg, between about 100 mg and about 200 mg, or between about 200 mg and about 300 mg. In some embodiments, the dose of the immunomodulator, e.g., an anti-PD-1 antibody or an antigen-binding fragment thereof, in the composition is from about 300 mg to about 500 mg, from about 300 mg to about 450 mg, from about 300 mg to about 400 mg, from about 300 mg to about 350 mg, from about 350 mg to about 500 mg, from about 400 mg to about 500 mg, or from about 450 mg to about 500 mg. In some embodiments, the amount of the immunomodulator, e.g., an anti-PD-1 antibody or antigen-binding fragment thereof, in the composition is at least about 80 mg, about 160 mg, or about 240 mg. In certain embodiments, the amount of the immunomodulator, e.g., an anti-PD-1 antibody or an antigen-binding fragment thereof, in the composition is at least about 360 mg or 480 mg. In some embodiments, the dose of the immunomodulator, e.g., an anti-PD-1 antibody or antigen-binding fragment thereof, in the composition is at least about 240 mg or at least about 80 mg. In one embodiment, the amount of the anti-PD-1 antibody or antigen-binding fragment thereof, in the composition is about 360 mg. In another embodiment, the amount of the immunomodulator, e.g., an anti-PD-1 antibody or antigen-binding fragment thereof, in the composition is about 480 mg. In some embodiments, the dose of the immunomodulator, e.g., an anti-PD-1 antibody or antigen-binding fragment thereof, in the composition is a least about 0.5 mg/kg, at least about 1 mg/kg, at least about 2 mg/kg, at least about 3 mg/kg or at least about 5 mg/kg. In some embodiments, the dose of the immunomodulator, e.g., an anti-PD-1 antibody or antigen-binding fragment thereof, in the composition is between about 0.5 mg/kg and about 5 mg/kg, between about 0.5 mg/kg and about 5 mg/kg, between about 0.5 mg/kg and about 3 mg/kg or between about 0.5 mg/kg and about 2 mg/kg. In some embodiments, the dose of the immunomodulator, e.g., an anti-PD-1 antibody or antigen-binding fragment thereof, in the composition is a least about 1 mg/kg. The corresponding dose of the half-life extended immune cell engaging protein is calculated using the desired ratio.

In some embodiments, the immunomodulator, e.g., an anti-PD-1 antibody or an antigen-binding fragment thereof, is administered at a subtherapeutic dose, such as, a dose of the therapeutic agent that is significantly lower than the usual or FDA-approved dose when administered as monotherapy for the treatment of the cancer. The quantity of the half-life extended immune cell engaging protein in the combination is calculated based on the desired ratio. For instance, dosages of Nivolumab that are lower than the typical 3 mg/kg, but not less than 0.001 mg/kg, are subtherapeutic dosages. The subtherapeutic doses of an anti-PD-1 antibody or antigen-binding fragment thereof used in the methods herein are higher than 0.001 mg/kg and lower than 3 mg/kg. In some embodiments, a subtherapeutic dose is about 0.001 mg/kg-about 1 mg/kg, about 0.01 mg/kg-about 1 mg/kg, about 0.1 mg/kg-about 1 mg/kg, or about 0.001 mg/kg-about 0.1 mg/kg body weight. In some embodiments, the subtherapeutic dose is at least about 0.001 mg/kg, at least about 0.005 mg/kg, at least about 0.01 mg/kg, at least about 0.05 mg/kg, at least about 0.1 mg/kg, at least about 0.5 mg/kg, or at least about 1.0 mg/kg body weight.

In some embodiments, a composition comprising the combination, or a composition comprising the immunomodulator, or a composition comprising the half-life extended immune cell engaging protein, is administered by intravenous infusion once about per week, once about every 2 weeks, once about every 3 weeks, or once about a month. In one embodiment, 360 mg of the immunomodulator, e.g., an anti-PD-1 antibody or antigen binding fragment is administered once every 3 weeks. In another embodiment, 480 mg of the immunomodulator, e.g., an anti-PD-1 antibody or antigen binding fragment is administered once about once every 4 weeks. In some embodiments, a composition comprising the combination, or a composition comprising the immunomodulator, or a composition comprising the half-life extended immune cell engaging protein is administered as an infusion, wherein the infusion occurs over at least about 10 minutes, about 20 minutes, about 30 minutes, about 45 minutes, about 60 minutes, about 90 minutes, about 2 hours, about 3 hours, about 4 hours or about 5 hours.

Actual dosage levels of the immunomodulator and the half-life extended immune cell engaging protein, in single or separate compositions, can be flat or varied so as to obtain an amount of the active ingredient which is effective to achieve the desired therapeutic response for a particular patient, composition, and mode of administration, without being unduly toxic to the patient. The selected dosage level for the immunomodulator, the half-life extended immune cell engaging protein, or a combination of both, will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure, comprising both immunomodulator and a half-life extended immune cell engaging protein in combination or individually, can be administered via one or more routes of administration using one or more of a variety of methods well known in the art. In some cases, the route and/or mode of administration varies depending upon the desired results.

In certain embodiments a combination as disclosed herein is used for a method of treating or ameliorating a proliferative disorder or condition, wherein the proliferative disorder or condition, e.g., the cancer, includes but is not limited to, a solid tumor, a soft tissue tumor (e.g., a hematological cancer, leukemia, lymphoma, or myeloma), and a metastatic lesion of any of the aforesaid cancers. In one embodiment, the cancer is a solid tumor. Examples of solid tumors include malignancies, e.g., sarcomas, adenocarcinomas, and carcinomas, of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma), and pancreas, as well as adenocarcinomas which include malignancies such as colon cancers, rectal cancer, renal-cell carcinoma, liver cancer, non-small cell lung cancer, cancer of the small intestine and cancer of the esophagus. The cancer may be at an early, intermediate, late stage or metastatic cancer.

In one embodiment, the cancer is chosen from a solid tumor, e.g., a lung cancer (e.g., a non-small cell lung cancer (NSCLC) (e.g., a NSCLC with squamous and/or non-squamous histology)), a colorectal cancer, a melanoma (e.g., an advanced melanoma), a head and neck cancer (e.g., head and neck squamous cell carcinoma (HNSCC), a digestive/gastrointestinal cancer, a gastric cancer, a neurologic cancer, a glioblastoma (e.g., glioblastoma multiforme), an ovarian cancer, a renal cancer, a liver cancer, a pancreatic cancer, a prostate cancer, a liver cancer; a breast cancer, an anal cancer, a gastro-esophageal cancer, a thyroid cancer, a cervical cancer; or a hematological cancer (e.g., chosen from a Hodgkin lymphoma, a non-Hodgkin lymphoma, a lymphocytic leukemia, or a myeloid leukemia).

In some embodiments, the cancer is selected from: mesothelioma, a prostate cancer, a breast cancer, a brain cancer, a bladder cancer, a pancreatic carcinoma, a renal cancer, a solid tumor, a liver cancer, a leiomyosarcoma, an endometrium cancer, a breast cancer, a female reproductive system cancer, an ovarian carcinoma, a soft tissue sarcoma, a gastric cancer, a digestive/gastrointestinal cancer, a colorectal cancer, a glioblastoma multiforme, a head and neck cancer, a squamous cell carcinoma, a colon cancer, a gastric cancer, a rhabdomyosarcoma, an adrenal cancer, a lung cancer, an esophageal cancer, a colon cancer, a lung cancer, a non-small cell lung carcinoma (NSCLC), a neuroblastoma, a melanoma, glioblastoma multiforme, an ovarian cancer, an endocrine cancer, a respiratory/thoracic cancer, an anal cancer, a gastro-esophageal cancer, a thyroid cancer, a cervical cancer, an endometrial cancer, a hematological cancer, a leukemia, a lymphocytic leukemia, a multiple myeloma, a lymphoma, a Hodgkin's lymphoma, a non-Hodgkin's lymphoma, a lymphocytic leukemia, an anaplastic large-cell lymphoma (ALCL), or a myeloid leukemia. In some embodiments, the cancer is an ovarian carcinoma, pancreatic carcinoma, mesothelioma, prostate cancer, or lung cancer.

Methods and combinations disclosed herein are useful for treating metastatic lesions associated with the aforementioned cancers. In other embodiments, the subject is a mammal, e.g, a primate, e.g., a higher primate, e.g, a human (e.g, a patient having, or at risk of having, a disorder described herein). In one embodiment, the subject to be treated is in need of enhancing an immune response. In one embodiment, the subject has, or is at risk of, having a disorder described herein, e.g., a cancer as described herein. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR, real-time quantitative RT-PCR, RNAseq, and the Nanostring platform (J. Clin. Invest. 2017; 127(8):2930-2940).

In some embodiments, administering an immune cell engaging protein as described herein results in an increased level or expression of an immune checkpoint protein, e.g., PD-1. In some embodiments, administering an immune cell engaging protein as described herein, e.g., a half-life extended immune cell engaging protein, increases the sensitivity of a subject to a therapy comprising administering an immunomodulator, e.g., an immune checkpoint inhibitor, e.g., an anti-PD-1 antibody. In some embodiments, administering an immune cell engaging protein as described herein improves the efficacy of a therapy comprising administering an immunomodulator to a subject. For instance, in some cases, administering an immune cell engaging protein (e.g., a half-life extended immune cell engaging protein) as described herein, increases the sensitivity of a non-responder subject to a therapy comprising administering an immunomodulator, e.g., an immune checkpoint inhibitor, e.g., an anti-PD-1 antibody, e.g., Pembrolizumab, Nivolumab. A “non-responder subject”, when referring to a specific anti-tumor response to treatment with a therapy, means the subject did not exhibit the anti-tumor response.

In some embodiments, the subject has previously been treated with an immunomodulator, e.g., an anti-PD-1 antibody. In some embodiments, the subject has or is identified as having a tumor that has one or more of high PD-L1 level or expression and/or Tumor Infiltrating Lymphocyte (TIL)+. In certain embodiments, the subject has or is identified as having a tumor that has high PD-L1 level or expression and TIL+. In some embodiments, the methods described herein further describe identifying a subject based on having a tumor that has one or more of high PD-L1 level or expression and/or TIL+. In certain embodiments, the methods described herein further describe identifying a subject based on having a tumor that has high PD-L1 level or expression and TIL+. In some embodiments, tumors that are TIL+ are positive for CD8 and IFNγ. In some embodiments, the subject has or is identified as having a high percentage of cells that are positive for one or more of PD-L1, CD8, and/or IFNγ. In certain embodiments, the subject has or is identified as having a high percentage of cells that are positive for all of PD-L1, CD8, and IFNγ. In some embodiments, the methods described herein further describe identifying a subject based on having a high percentage of cells that are positive for one or more of PD-L1, CD8, and/or IFNγ. In certain embodiments, the methods described herein further describe identifying a subject based on having a high percentage of cells that are positive for all of PD-L1, CD8, and IFNγ. In some embodiments, the subject has or is identified as having one or more of PD-L1, CD8, and/or IFNγ, and one or more of a lung cancer, e.g., squamous cell lung cancer or lung adenocarcinoma; a head and neck cancer; a squamous cell cervical cancer; a stomach cancer; a thyroid cancer; and/or a melanoma. In certain embodiments, the methods described herein further describe identifying a subject based on having one or more of PD-L1, CD8, and/or IFNγ, and one or more of a lung cancer, e.g., squamous cell lung cancer or lung adenocarcinoma; a head and neck cancer; a squamous cell cervical cancer; a stomach cancer; a thyroid cancer; and/or a melanoma.

### Compositions, Dosages, and Administration

Disclosed herein, as described above, are combinations comprising an immunomodulator, a half-life extended immune cell engaging protein, and optionally one or more additional therapeutic agents. Each therapeutic agent in a combination (e.g., an immunomodulator, a half-life extended immune cell engaging protein, and optionally one or more additional therapeutic agents), in some embodiments, is administered either alone or in a medicament (also referred to herein as a composition or a pharmaceutical composition) which comprises the therapeutic agent and one or more pharmaceutically acceptable carriers, excipients and diluents, according to standard pharmaceutical practice. The combinations provided herein, in some embodiments, are administered during periods of active disorder, or during a period of remission or less active disease. The combination, in some embodiments, are administered before another treatment, concurrently with another treatment, post-treatment, or during remission of the disorder.

Each therapeutic agent in a combination (e.g., an immunomodulator, a half-life extended immune cell engaging protein, and optionally one or more additional therapeutic agents), in some embodiments, is administered simultaneously (e.g., in the same medicament), concurrently (e.g., in separate medicaments administered one right after the other in any order) or sequentially in any order. Sequential administration is particularly useful when the therapeutic agents in the combination are in different dosage forms (e.g., one agent is atablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks. In some embodiments, concurrent administration of two therapeutic agents does not require that the agents be administered at the same time or by the same route, as long as there is an overlap in the time period during which the agents are exerting their therapeutic effect. In some embodiments, simultaneous or sequential administration is contemplated, as is administration on different days or weeks.

Dosages and therapeutic regimens for the combinations as described herein, in some instances, are determined by a skilled artisan. For instance, in some embodiments, the combination comprising an immunomodulator, a half-life extended immune cell engaging protein as described herein is administered to a subject systemically (e.g., orally, parenterally, subcutaneously, intravenously, rectally, intramuscularly, intraperitoneally, intranasally, transdermally, or by inhalation or intracavitary installation), topically, or by application to mucous membranes, such as the nose, throat and bronchial tubes.

In certain embodiments, an immunomodulator is an anti-PD-1 antibody, and the anti-PD-1 antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule, in some embodiments, varies, e.g., once a week to once every 2, 3, or 4 weeks. In one embodiment, the anti-PD-1 antibody molecule is administered at a dose from about 10 to 20 mg/kg every other week.

In one embodiment, the anti-PD-1 antibody molecule, e.g., Nivolumab or Pembrolizumab, is administered intravenously at a dose from about 1 mg/kg to 3 mg/kg, e.g., about 1 mg/kg, 2 mg/kg or 3 mg/kg, every two weeks. In one embodiment, the anti-PD-1 antibody molecule, e.g., Nivolumab, is administered intravenously at a dose of about 2 mg/kg at 3-week intervals. In one embodiment, Nivolumab or Pembrolizumab is administered in an amount from about 1 mg/kg to 5 mg/kg, e.g., 3 mg/kg, and may be administered over a period of 60 minutes, such as once a week to once every 2, 3 or 4 weeks.

In some embodiments of the combination, the immunomodulator is administered intravenously. In some embodiments of the combination, the half-life extended immune cell engaging protein, is administered intravenously. In some embodiments of the combination, the immunomodulator (e.g., an anti-PD-1 antibody molecule) is administered, e.g., intravenously, at least one, two, three, four, five, six, or seven days, e.g., three days, after a half-life extended immune cell engaging protein is administered, e.g., intravenously. In some embodiments of the combination, the immunomodulator (e.g., an anti-PD-1 antibody molecule) is administered, e.g., intravenously, at least one, two, three, four, five, six, or seven days, e.g., three days, before a half-life extended immune cell engaging protein is administered, e.g., intravenously. In some embodiments of the combination, the immunomodulator (e.g., an anti-PD-1 antibody molecule) is administered, e.g., intravenously, on the same day, as a half-life extended immune cell engaging protein is administered, e.g., intravenously. In some embodiments of a combination, the administration of the immunomodulator (e.g., an anti-PD-1 antibody molecule) and the half-life extended immune cell engaging protein results in enhanced reduction of a cancer/carcinoma, e.g., pancreatic carcinoma, ovarian carcinoma, prostate cancer, lung cancer, mesothelioma, relative to administration of each of these agents as a monotherapy. In certain embodiments, in a combination, the concentration of a half-life extended immune cell engaging protein, that is required to achieve inhibition, e.g., growth inhibition/tumor regression, is lower than the therapeutic dose of the agent as a monotherapy, e.g., 10-20%, 20-30%, 30-40%, 40-50%, 50-60%, 60-70%, 70-80%, or 80-90% lower. In certain embodiments, in a combination, the concentration of an immunomodulator, that is required to achieve inhibition, e.g., growth inhibition/tumor regression, is lower than the therapeutic dose of the agent as a monotherapy, e.g., 10-20%, 20-30%, 30-40%, 40-50%, 50-60%, 60-70%, 70-80%, or 80-90% lower.

In some embodiments of the combinations described herein, the immune cell engaging protein (e.g., a half-life extended immune cell engaging protein) is administered at a dosage of from about 0.5 ng/kg to about 500 ng/kg; e.g., from about 1 ng/kg to about 400 ng/kg, from about 2 ng/kg to about 300 ng/kg, from about 4 ng/kg to about 200 ng/kg, from about 8 ng/kg to about 100 ng/kg; from about 1 ng/kg to about 200 ng/kg, from about 1.3 ng/kg to about 160 ng/kg.

The combinations as described herein, in some embodiments, are used in combination with additional agents or therapeutic modalities. The combination therapies can be administered simultaneously or sequentially in any order. Any combination and sequence of the anti-PD-1 or PD-L1 antibody molecules and other therapeutic agents, procedures or modalities (e.g., as described herein) can be used

Combinations with Additional Therapeutic Agents

In certain embodiments, the combination of described herein are administered in combination with one or more of other antibody molecules, chemotherapy, other anti-cancer therapy (e.g., targeted anti-cancer therapies, gene therapy, viral therapy, RNA therapy bone marrow transplantation, nanotherapy, or oncolytic drugs), cytotoxic agents, immune-based therapies (e.g., cytokines or cell-based immune therapies), surgical procedures (e.g., lumpectomy or mastectomy) or radiation procedures, or a combination of any of the foregoing. The additional therapy may be in the form of adjuvant or neoadjuvant therapy. In some embodiments, the additional therapy is an enzymatic inhibitor (e.g., a small molecule enzymatic inhibitor) or a metastatic inhibitor. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation (e.g., gamma irradiation). In other embodiments, the additional therapy is surgery or radiation, or a combination thereof. In other embodiments, the additional therapy is a therapy targeting an mTOR pathway, an HSP90 inhibitor, or a tubulin inhibitor.

In some embodiments, the combinations described herein are administered in combination with a chemotherapeutic agent. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; cally statin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e g., Agnew, Chem. Inti. Ed. Engl., 33: 183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti androgens such as flutamide, nilutamide, bicalutamide, leuprolide, and goserelin; and pharmaceutically acceptable salts, acids or derivatives of any of the above.

In some embodiments, provided herein is a combination further comprising one or more additional therapeutic agents, and methods to treat a cancer using such a combination of an immunomodulator, a half-life extended immune cell engaging protein and an additional therapeutic agent. Non-limiting examples of such additional therapeutic agents include: a c-MET inhibitor, an Alk inhibitor, a CDK4/6 inhibitor, a PI3K-inhibitor, a BRAF inhibitor, a CAR T cell targeting CD19, a MEK inhibitor, a BCR-ABL inhibitor, or any combination thereof.

In one embodiment, the c-MET inhibitor is INC280 (formerly known as INCB28060). In one embodiment, the immunomodulator is an anti-PD-1 antibody (e.g., Nivolumab, Pembrolizumab or MSB0010718C), and the additional therapeutic agent is INC280, and the combination with a half-life extended immune cell engaging protein is used is a method of treating a cancer, e.g., a lung cancer (e.g., non-small cell lung cancer (NSCLC)), glioblastoma multiforme (GBM), a renal cancer, a liver cancer (e.g., a hepatocellular carcinoma) or a gastric cancer. In some embodiments, the cancer has, or is identified as having, a c-MET mutation (e.g., a c-MET mutation or a c-MET amplification). In certain embodiments, INC280 is administered at an oral dose of about 100 to 1000 mg, e.g., about 200 mg to 900 mg, about 300 mg to 800 mg, or about 400 mg to 700 mg, e.g., about 400 mg, 500 mg or 600 mg. The dosing schedule can vary from e.g., every other day to daily, twice or three times a day. In one embodiment, INC280 is administered at an oral dose from about 400 to 600 mg twice a day.

In one embodiment, the Alk inhibitor is LDK378 (also known as ceritinib (Zykadia®). In one embodiment, the immunomodulator is an anti-PD-1 antibody (e.g., Nivolumab, Pembrolizumab or MSB0010718C), and the additional therapeutic agent is LDK378, and the combination with a half-life extended immune cell engaging protein is used is a method of treating a cancer, e.g., a solid tumor, e.g., a lung cancer (e.g., non-small cell lung cancer (NSCLC)), a lymphoma (e.g., an anaplastic large-cell lymphoma or non-Hodgkin lymphoma), an inflammatory myofibroblastic tumor (IMT), or a neuroblastoma. In some embodiments, the NSCLC is a stage IIIB or IV NSCLC, or a relapsed locally advanced or metastic NSCLC. In some embodiments, the cancer (e.g., the lung cancer, lymphoma, inflammatory myofibroblastic tumor, or neuroblastoma) has, or is identified as having, an ALK rearrangement or translocation, e.g., an ALK fusion. In one embodiment, the ALK fusion is an EML4-ALK fusion, e.g., an EML4-ALK fusion. In another embodiment, the ALK fusion is an ALK-ROS1 fusion. In certain embodiments, the cancer has progressed on, or is resistant or tolerant to, a ROS1 inhibitor, or an ALK inhibitor, e.g., an ALK inhibitor other than LDK378. In some embodiments, the cancer has progressed on, or is resistant or tolerant to, crizotinib. In one embodiment, the subject is an ALK-naive patient, e.g., a human patient. In another embodiment, the subject is apatient, e.g., a human patient, that has been pre-treated with an ALK inhibitor. In another embodiment, the subject is a patient, e.g., a human patient, that has been pretreated with LDK378. In one embodiment, the half-life extended immune cell engaging protein, the LDK378 and the anti-PD-1 antibody (e.g., Nivolumab, Pembrolizumab or MSB0010718C) are administered to an ALK-naive patient. In another embodiment, the half-life extended immune cell engaging protein, the LDK378 and the anti-PD-1 antibody (e.g., Nivolumab, Pembrolizumab or MSB0010718C) are administered to a patient that has been pretreated with an ALK inhibitor. In yet another embodiment, the half-life extended immune cell engaging protein, the LDK378 and the anti-PD-1 antibody (e.g., Nivolumab, Pembrolizumab or MSB0010718C) are administered to a patient that has been pretreated with LDK378. In certain embodiments, LDK378 is administered at an oral dose of about 100 to 1000 mg, e.g., about 150 mg to 900 mg, about 200 mg to 800 mg, about 300 mg to 700 mg, or about 400 mg to 600 mg, e.g., about 150 mg, 300 mg, 450 mg, 600 mg or 750 mg. In certain embodiment, LDK378 is administered at an oral dose of about 750 mg or lower, e.g., about 600 mg or lower, e.g., about 450 mg or lower. In certain embodiments, LDK378 is administered with food. In other embodiments, the dose is under fasting condition. The dosing schedule can vary from e.g., every other day to daily, twice or three times a day. In one embodiment, LDK378 is administered daily. In one embodiment, LDK378 is administered at an oral dose from about 150 mg to 750 mg daily, either with food or in a fasting condition. In one embodiment, LDK378 is administered at an oral dose of about 750 mg daily, in a fasting condition. In one embodiment, LDK378 is administered at an oral dose of about 750 mg daily, via capsule or tablet. In another embodiment, LDK378 is administered at an oral dose of about 600 mg daily, via capsule or tablet. In one embodiment, LDK378 is administered at an oral dose of about 450 mg daily, via capsule or tablet. In one embodiment, LDK378 is administered at a dose of about 450 mg and the anti-PD-1 antibody (e.g., Nivolumab, Pembrolizumab or MSB0010718C) is administered at a dose of about 3 mg/kg. In another embodiment, the LDK378 dose is 600 mg and the anti-PD-1 antibody (e.g., Nivolumab, Pembrolizumab or MSB0010718C) dose is 3 mg/kg. In one embodiment, LDK378 is administered with a low fat meal.

In one embodiment, the CDK4/6 inhibitor is LEE011 (also known as Ribociclib®). In one embodiment, the immunomodulator is an anti-PD-1 antibody (e.g., Nivolumab, Pembrolizumab or MSB0010718C), and the additional therapeutic agent is LEE011, and the combination with a half-life extended immune cell engaging protein is used is a method of treating a cancer, e.g., a solid tumor, e.g., a lung cancer (e.g., non-small cell lung cancer (NSCLC)), a neurologic cancer, melanoma or a breast cancer, or a hematological malignancy, e.g., a lymphoma.

In one embodiment, the PI3K inhibitor is BKM120 or BYL719. In one embodiment, the immunomodulator is an anti-PD-1 antibody (e.g., Nivolumab, Pembrolizumab or MSB0010718C), and the additional therapeutic agent is BKM120 or BYL719, and the combination with a half-life extended immune cell engaging protein is used is a method of treating a cancer or a disorder, e.g., a solid tumor, e.g., a lung cancer (e.g., non-small cell lung cancer (NSCLC)), a prostate cancer, an endocrine cancer, an ovarian cancer, a melanoma, a bladder cancer, a female reproductive system cancer, a digestive/gastrointestinal cancer, a colorectal cancer, glioblastoma multiforme (GBM), a head and neck cancer, a gastric cancer, a pancreatic cancer or a breast cancer; or a hematological malignancy, e.g., leukemia, non-Hodgkin lymphoma; or a hematopoiesis disorder.

In one embodiment, the BRAF inhibitor is LGX818. In one embodiment, the immunomodulator is an anti-PD-1 antibody (e.g., Nivolumab, Pembrolizumab or MSB0010718C), and the additional therapeutic agent is LGX818, and the combination with a half-life extended immune cell engaging protein is used is a method of treating a cancer, e.g., a solid tumor, e.g., a lung cancer (e.g., non-small cell lung cancer (NSCLC)), a melanoma, e.g., advanced melanoma, a thyroid cancer, e.g., papillary thyroid cancer, or a colorectal cancer. In some embodiments, the cancer has, or is identified as having, a BRAF mutation (e.g., a BRAF V600E mutation), a BRAF wildtype, a KRAS wildtype or an activating KRAS mutation. In some embodiments, the cancer is at an early, intermediate or late stage.

In one embodiment, the additional therapeutic agent is a CAR T cell targeting CD19. In one embodiment, the immunomodulator is an anti-PD-1 antibody (e.g., Nivolumab, Pembrolizumab or MSB0010718C), and the additional therapeutic agent is the CAR T cell targeting CD19 (e.g., CTL019), and the combination with a half-life extended immune cell engaging protein is used is a method of treating a cancer, a solid tumor, or a hematological malignancy, e.g., a lymphocytic leukemia or a non-Hodgkin lymphoma. In one embodiment, the CAR T cell targeting CD19 has the USAN designation TISAGENLECLEUCEL-T. CTLO19 is made by a gene modification of T cells is mediated by stable insertion via transduction with a self-inactivating, replication deficient Lentiviral (LV) vector containing the CTLO19 transgene under the control of the EF-1 alpha promoter. CTLO19 is a mixture of transgene positive and negative T cells that are delivered to the subject on the basis of percent transgene positive T cells.

In one embodiment, the additional therapeutic agent is the MEK inhibitor (e.g., MEK162). In one embodiment, the immunomodulator is an anti-PD-1 antibody (e.g., Nivolumab, Pembrolizumab or MSB0010718C), and the additional therapeutic agent is MEK162, and the combination with a half-life extended immune cell engaging protein is used is a method of treating a cancer or a disorder, e.g., a melanoma, a colorectal cancer, a non-small cell lung cancer, an ovarian cancer, a breast cancer, a prostate cancer, a pancreatic cancer, a hematological malignancy or a renal cell carcinoma, a multisystem genetic disorder, a digestive/gastrointestinal cancer, a gastric cancer, or a colorectal cancer; or rheumatoid arthritis. In some embodiments, the cancer has, or is identified as having, a KRAS mutation.

In one embodiment, the additional therapeutic agent is the BCR-ABL inhibitor (e.g., AMN-107 (also known as Nilotinib, trade name Tasigna). In one embodiment, the immunomodulator is an anti-PD-1 antibody (e.g., Nivolumab, Pembrolizumab or MSB0010718C), and the additional therapeutic agent is AMN-107, and the combination with a half-life extended immune cell engaging protein is used is a method of treating a cancer or a disorder, e.g., a solid tumor, e.g., a neurologic cancer, a melanoma, a digestive/gastrointestinal cancer, a colorectal cancer, a head and neck cancer; or a hematological malignancy, e.g., chronic myelogenous leukemia (CML), a lymphocytic leukemia, a myeloid leukemia; Parkinson's disease; or pulmonary hypertension.

In one embodiment, the additional therapeutic agent is a small molecule PD-1/PD-L1 antagonist. In one embodiment, the small molecule PD-1/PD-L1 antagonist is PDI-1 (PD1/PD-L1 inhibitor 1) as described in Wang et al., A Small Molecule Antagonist of PD-1/PD-L1 Interactions Acts as an Immune Checkpoint Inhibitor for NSCLC and Melanoma Immunotherapy. Front. Immunol. 12:654463. doi: 10.3389/fimmu.2021.654463. In one embodiment, the small molecule PD-1/PD-L1 antagonist is a bioactive macrocyclic peptide as described in Magiera-Mularz et al. Bioactive macrocyclic inhibitors of the PD-1/PD-L1 immune checkpoint Angew. Chem. Int. Ed. 10.1002/anie.201707707.

### EXAMPLES

### Example 1: PSMA Targeting TriTAC Affects PD-1/PD-L1 Expression

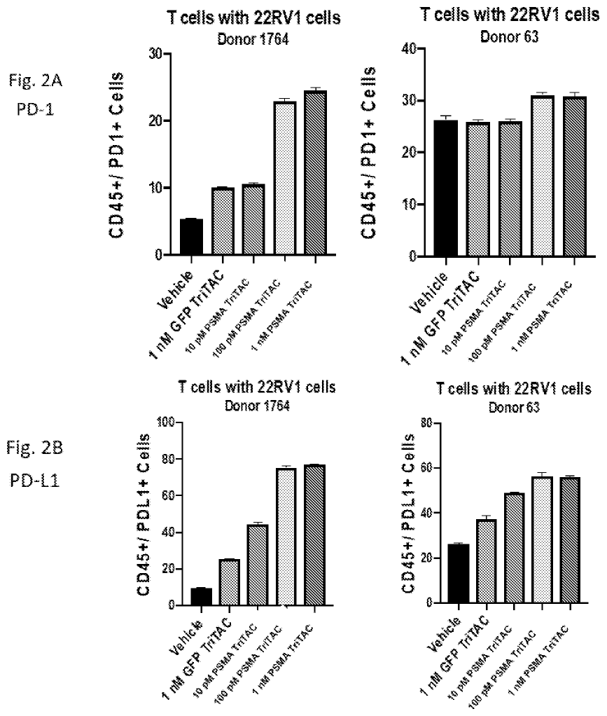

T cells from two donors were co-cultured with 22Rv1 prostate cancer cells at a ratio of 10:1 and treated with PSMA targeting TriTAC (SEQ ID NO: 3340) at 10 μM, 100 μM, and 1 nM, GFP targeting TriTAC or vehicle for 48 hrs. Expression levels of PD-1 and PD-L1 were then measured by fluorescence activated cell sorting (FACS) analysis. FIG. 2A demonstrates the change in PD-1 expression levels after PSMA targeting TriTAC treatment. FIG. 2B demonstrates the change in PD-L1 expression levels after PSMA targeting TriTAC treatment.

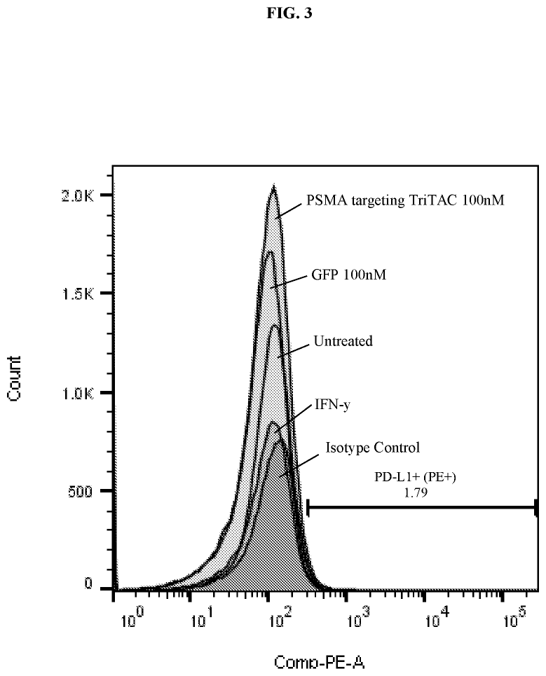

22Rv1 cancer cells were treated with 100 units/mL IFNγ or were co-cultured with resting T cells from a healthy donor then treated with PSMA targeting TriTAC for 48 hours. Expression levels of PD-L1 was then measured by FACS analysis. FIG. 3 demonstrates the PD-L1 levels after treatment with PSMA targeting TriTAC or IFNγ for 22Rv1 cancer cells.

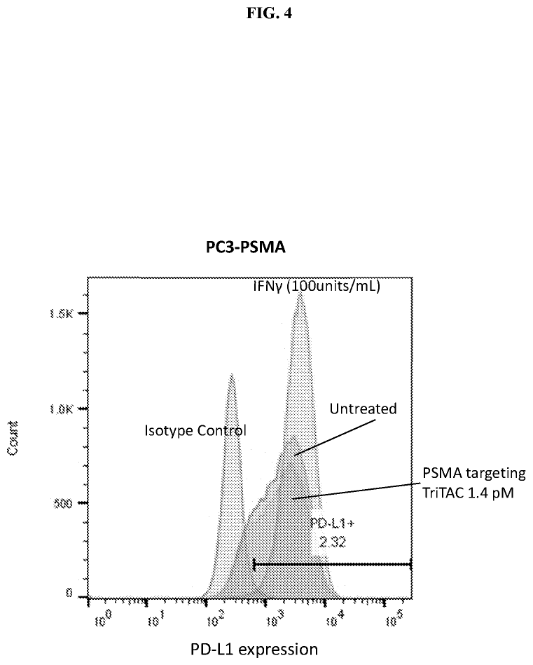

PC3 cells were engineered to express PSMA and are called PC3-PSMA cells. PC3-PSMA cancer cells were treated 100 units/mL IFNγ or were co-cultured with resting T cells from a healthy donor then treated with PSMA targeting TriTAC for 48 hours. Expression of PD-L1 was then measured by FACS analysis. FIG. 4 demonstrates the PD-L1 levels after treatment with PSMA targeting TriTAC or IFNγ for PC3-PSMA cancer cells.

### Example 2: In Vivo Antitumor Activity Study of PSMA Targeting TriTAC in Combination with PD-1/PD-L1 Inhibitors

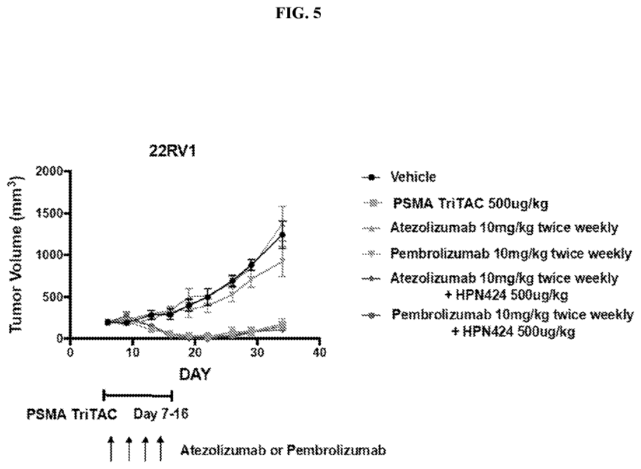

22Rv1 prostate cancer model: NOD.Cg-Prkd^{scid} Il2rg^{tm1Wjl}/SzJ were subcutaneously implanted with a mixture of 22Rv1 prostate cancer cells and T cells at a ratio of 1:1 of 5×10⁶ cells each. Mice were randomized on day 6 when tumors reached ˜199 mm³. Treatment was initiated the following day on days 7-16 by intraperitoneal (i.p.) injection of vehicle, 500 μg/kg PSMA targeting TriTAC alone once a day, 10 mg/kg Atezolizumab alone twice weekly, 10 mg/kg Pembrolizumab alone twice weekly, 10 mg/kg Atezolizumab twice weekly in combination with 500 μg/kg PSMA targeting TriTAC once a day, or 10 mg/kg Pembrolizumab twice weekly in combination with 500 μg/kg PSMA targeting TriTAC once a day. Five mice were used for each treatment group. FIG. 5 demonstrates the tumor volumes under each treatment for 22Rv1 prostate cancer cells.

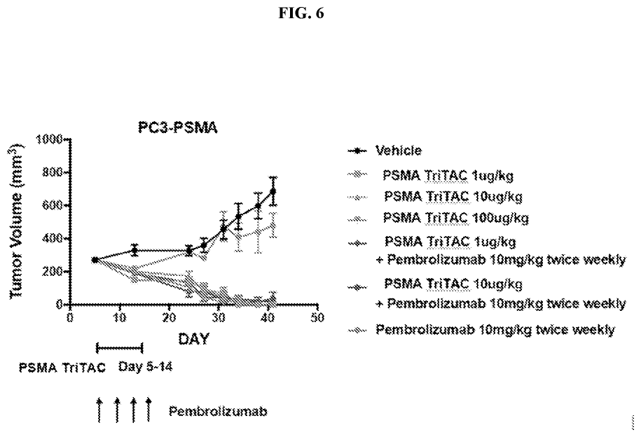

PC3-PSMA cancer model: NOD.Cg-Prkd^{scid} Il2rg^{tm1Wjl}/SzJ were subcutaneously implanted with a mixture of PC3-PSMA cancer cells and T cells at a ratio of 2:1 (10×10⁶ PC3-PSMA: 5×10⁶ T cells). Mice were randomized on day 5 when tumors reached ˜270 mm³. Treatment was initiated the following day on days 5-14 by intraperitoneal (i.p.) injection of vehicle, 1 μg/kg PSMA targeting TriTAC alone once a day, 10 μg/kg PSMA targeting TriTAC alone once a day, 100 μg/kg PSMA targeting TriTAC alone once a day, 10 mg/kg Pembrolizumab alone twice weekly, 10 mg/kg Pembrolizumab twice weekly in combination with 1 μg/kg PSMA targeting TriTAC once a day, or 10 mg/kg Pembrolizumab twice weekly in combination with 10 μg/kg PSMA targeting TriTAC once a day. Eight mice were used for each treatment group. FIG. 6 demonstrates the tumor volumes under each treatment for PC3-PSMA cancer cells.

### Example 3: MSLN Targeting TriTAC Affects PD-1/PD-L1 Expression

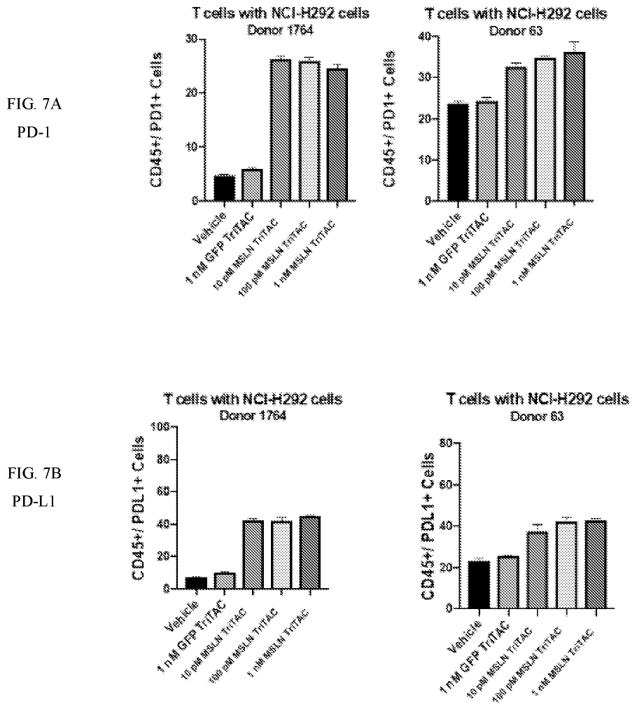

T cells from two donors were co-cultured with NCI-H292 lung cancer cells at a ratio of 10:1 and treated with MSLN targeting TriTAC (SEQ ID NO: 3376) at 10 μM, 100 μM, and 1 nM, GFP targeting TriTAC or vehicle for 48 hrs. Expression levels of PD-1 and PD-L1 were then measured by FACS analysis. FIG. 7A demonstrates the change in PD-1 expression levels after MSLN targeting TriTAC treatment. FIG. 7B demonstrates the change in PD-L1 expression levels after MSLN targeting TriTAC treatment.

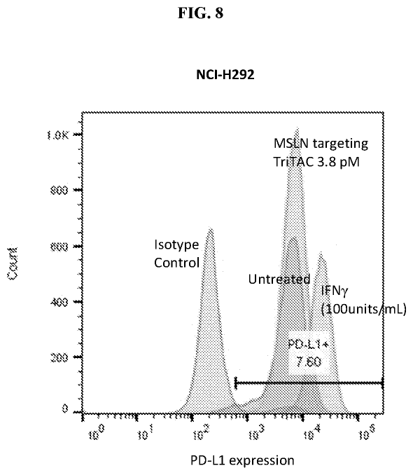

NCI-H292 lung cancer cells were treated 100 units/mL IFNγ or were co-cultured with resting T cells from a healthy donor then treated with MSLN targeting TriTAC for 48 hours. Expression levels of PD-L1 was then measured by FACS analysis. FIG. 8 demonstrates the PD-L1 levels after treatment with MSLN targeting TriTAC or IFNγ for NCI-H292 lung cancer cells.

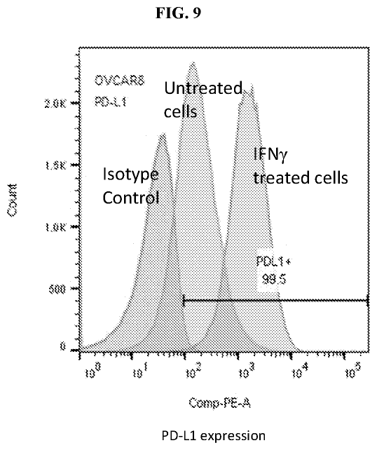

OVCAR8 ovarian cancer cells were treated 100 units/mL IFNγ for 48 hours. Expression levels of PD-L1 was then measured by FACS analysis. FIG. 9 demonstrates the PD-L1 levels after treatment with IFNγ for OVCAR8 ovarian cancer cells.

### Example 4: In Vivo Antitumor Activity Study of MSLN Targeting TriTAC in Combination with PD-1/PD-L1 Inhibitors

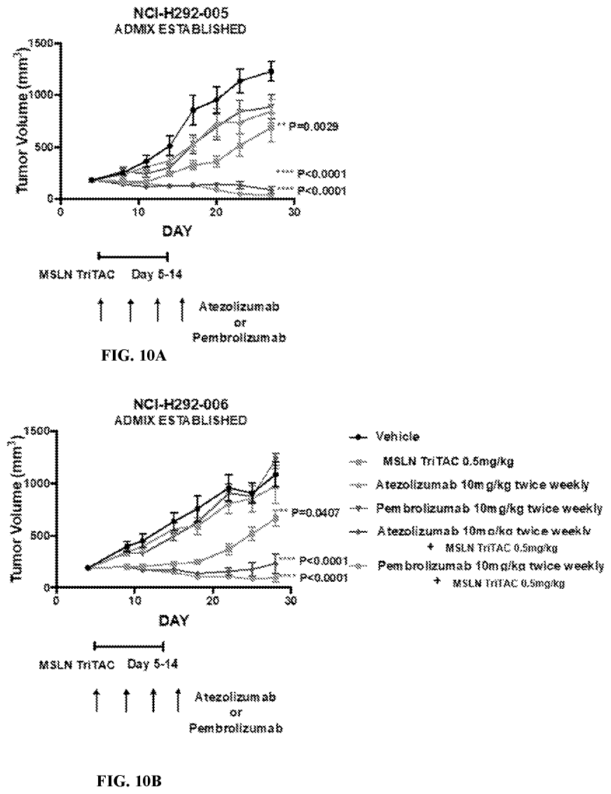

NCI-H292 cancer model: NOD.Cg-Prkd^{scid} Il2rg^{tm1Wjl}/SzJ were subcutaneously implanted with a mixture of NCI-H292 and T cells at a ratio of 1:1 of 5×10⁶ cells each. Mice were randomized on day 4 when tumors reached 180 mm³. Treatment was initiated the following day on days 5-14 by i.p. injection of vehicle, 0.5 mg/kg MSLN targeting TriTAC alone once a day, 10 mg/kg Atezolizumab alone twice weekly (on days 5, 9, 12, 16), 10 mg/kg Pembrolizumab alone twice weekly (on days 5, 9, 12, 16), 10 mg/kg Atezolizumab in combination with 0.5 mg/kg MSLN targeting TriTAC once a day, or 10 mg/kg Pembrolizumab in combination with 0.5 mg/kg MSLN targeting TriTAC once a day. Ten mice were used for each treatment group. FIG. 10 demonstrates the tumor volumes under each treatment for NCI-H292 cancer cells.

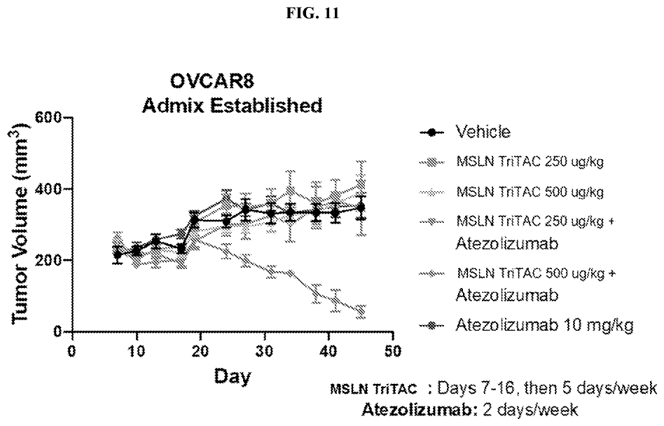

OVCAR8 ovarian cancer model: NOD.Cg-Prkd^{scid} Il2rg^{tm1Wjl}/SzJ were subcutaneously implanted with a mixture of OVCAR8 and T cells at a ratio of 2:1 (10×10⁶ OVCAR8: 5×10⁶ T cells). Mice were randomized on day 6 when tumors reached about 220 mm³. Treatment was initiated the following day by i.p. injection of vehicle; 250 μg/kg MSLN targeting TriTAC alone once a day for days 7-16, then 5 days/week for the duration of the study; 500 μg/kg MSLN targeting TriTAC alone once a day for days 7-16, then 5 days/week for the duration of the study; 10 mg/kg Atezolizumab alone twice weekly; 10 mg/kg Atezolizumab twice weekly in combination with 250 μg/kg MSLN targeting TriTAC once a day for days 7-16, then 5 days/week for the duration of the study; 10 mg/kg Atezolizumab twice weekly in combination with 250 μg/kg MSLN targeting TriTAC once a day for days 7-16, then 5 days/week for the duration of the study. Eight mice were used for each treatment group. FIG. 11 demonstrates the tumor volumes under each treatment for OVCAR8 ovarian cancer cells.

### Example 5: DLL3 Targeting TriTAC Affects PD-1/PD-L1 Expression

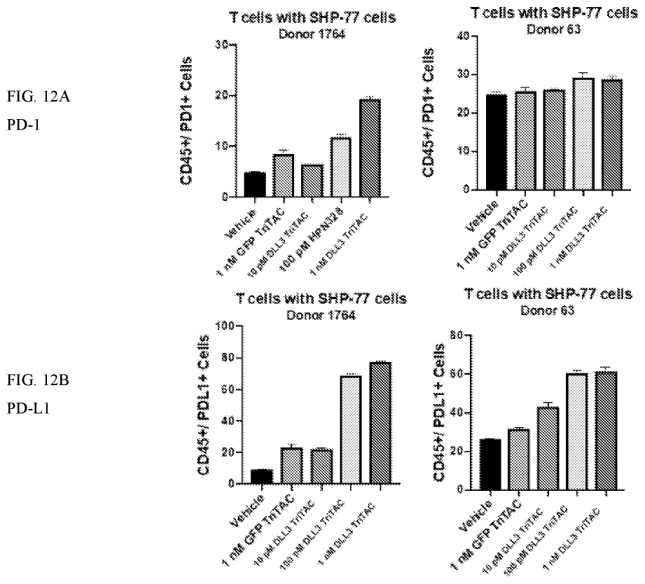

T cells from two donors were co-cultured with SHP-77 small cell lung cancer cells at a ratio of 10:1 and treated with DLL3 targeting TriTAC (SEQ ID NO: 3461) at 10 pM, 100 pM, and 1 nM, GFP targeting TriTAC or vehicle for 48 hrs. Expression levels of PD-1 and PD-L1 were then measured by FACS analysis. FIG. 12A demonstrates the change in PD-1 expression levels after DLL3 targeting TriTAC treatment. FIG. 12B demonstrates the change in PD-L1 expression levels after DLL3 targeting TriTAC treatment.

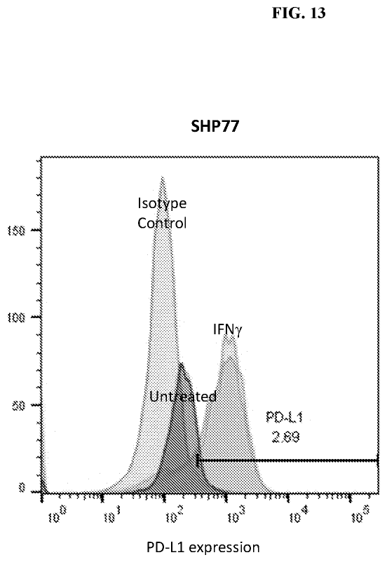

SHP-77 small cell lung cancer cells express elevated PD-L1 in response to IFNγ treatment in vitro: SHP-77 small cell lung cancer cells were treated with 100 units/mL IFNγ for 48 hours. Expression of PD-L1 was then measured by FACS analysis. FIG. 13 demonstrates the PD-L1 levels after treatment with IFNγ for SHP-77 small cell lung cancer cells.

### Example 6: In Vivo Antitumor Activity Study of DLL3 Targeting TriTAC in Combination with PD-1/PD-L1 Inhibitors

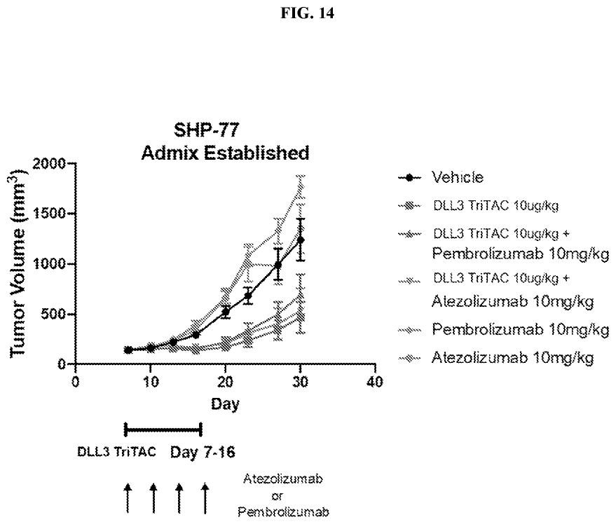

SHP-77 small cell lung cancer tumor model: NOD.Cg-Prkd^{scid} Il2rg^{tm1Wjl}/SzJ were subcutaneously implanted with a mixture of SHP-77 and T cells at a ratio of 2:1 (10×10⁶ SHP-77: 5×10⁶ T cells). Mice were randomized on day 7 when tumors reached ˜141 mm³. Treatment was initiated the following day on days 7-16 by i.p. injection of vehicle, 10 μg/kg DLL3 targeting TriTAC alone once a day, 10 mg/kg Pembrolizumab alone twice weekly, 10 mg/kg Atezolizumab alone twice weekly, 10 mg/kg Pembrolizumab twice weekly in combination with 10 μg/kg DLL3 targeting TriTAC once a day or 10 mg/kg Atezolizumab twice weekly in combination with 10 μg/kg DLL3 targeting TriTAC once a day. Eight mice were used for each treatment group. FIG. 14 demonstrates the tumor volumes under each treatment for SHP-77 small cell lung cancer cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Lengthy table referenced here
US20240084035A1-20240314-T00001
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20240084035A1). An electronic copy of the table will also be available from the USPTO upon request and payment of the fee set forth in 37 CFR 1.19(b)(3).

1. A combination comprising: an immunomodulator and a half-life extended immune cell engaging protein.

2. The combination of claim 1, wherein the half-life extended immune cell engaging protein comprises an immune cell engaging domain.

3. The combination of claim 2, wherein the immune cell engaging domain comprises a natural killer (NK) cell engaging domain, a T cell engaging domain, a B cell engaging domain, a dendritic cell engaging domain, a macrophage cell engaging domain, or a combination thereof.

4. The combination of claim 3, wherein the immune cell engaging domain comprises the T cell engaging domain.

5. The combination of claim 4, wherein the T cell engaging domain binds a CD3 molecule.

6. The combination of claim 5, wherein the CD3 molecule is at least one of: a CD3γ molecule, a CD3δ molecule, or a CD3ε molecule.

7. The combination of claim 1, wherein the immunomodulator comprises an immunostimulatory antibody agonist of a co-stimulatory receptor.

8. The combination of claim 1, wherein the immunomodulator comprises an immune checkpoint modulator.

9. The combination of claim 8, wherein the immune checkpoint modulator is an antagonist of at least one of: programmed cell death 1 (PDCD1, PD1, PD-1), CD274 (CD274, PDL1, PD-L1), PD-L2, cytotoxic T-lymphocyte associated protein 4 (CTLA4, CD152), CD276 (B7H3); V-set domain containing T cell activation inhibitor 1 (VTCN1, B7H4), CD272 (B and T lymphocyte associated (BTLA)), killer cell immunoglobulin like receptor, three Ig domains and long cytoplasmic tail 1 (KIR, CD158E1), lymphocyte activating 3 (LAG3, CD223), hepatitis A virus cellular receptor 2 (HAVCR2, TIMD3, TIM3), V-set immunoregulatory receptor (VSIR, B7H5, VISTA), T cell immunoreceptor with Ig and ITIM domains (TIGIT), programmed cell death 1 ligand 2 (PDCD1LG2, PD-L2, CD273), immunoglobulin superfamily member 11 (IGSF11, VSIG3), TNFRSF14 (HVEM, CD270), TNFSF14 (HVEML), PVR related immunoglobulin domain containing (PVRIG, CD112R), galectin 9 (LGALS9), killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1), killer cell lectin like receptor C1 (KLRC1, NKG2A, CD159A), killer cell lectin like receptor D1 (KLRD1, CD94), killer cell lectin like receptor G1 (KLRG1, CLEC15A, MAFA, 2F1), sialic acid binding Ig like lectin 7 (SIGLEC7), SIGLEC, sialic acid binding Ig like lectin 9 (SIGLEC9), CEACAM (e.g., CEACAM-1, CEACAM-3 and/or CEACAM-5), VISTA, LAIR1, CD160, 2B4, CD80, CD86, B7-H1, B7-H3 (CD276), B7-H4 (VTCN1), HVEM (TNFRSF14 or CD270), KIR, A2AR, A2BR, MHC class I, MHC class II, GAL9, adenosine, TGFR (e.g., TGFR beta), CD94/NKG2A, IDO, TDO, CD39, CD73, GARP, CD47, PVRIG, CSF1R, and NOX, or any combination thereof.

10. The combination of claim 8, wherein the immune checkpoint modulator is an antagonist of PD-1, and the antagonist of PD-1 is selected from the group consisting of: Pembrolizumab, Pidilizumab (CT-011), Spartalizumab (PDR001), Nivolumab (BMS-936558, MDX-1106), MEDI0680 (AMP-514), Cemiplimab (REGN2810), Dostarlimab (TSR-042), Sasanlimab (PF-06801591), Tislelizumab (BGB-A317), BGB-108, Tislelizumab (BGB-A317), Camrelizumab (INCSHR1210, SHR-1210), AMP-224, Zimberelimab (AB122, GLS-010, WBP-3055), AK-103 (HX-008), AK-105 (anti-PD-1 antibody), CS1003, HLX10, Retifanlimab (MGA-012), BI-754091, Balstilimab (AGEN2034), toripalimab (JS-001), cetrelimab (JNJ-63723283), genolimzumab (CBT-501), LZM009, Prolgolimab (BCD-100), Sym021, ABBV-181, BAT-1306, JTX-4014, sintilimab (IBI-308), Tebotelimab (MGD013), MGD-019, KN-046, MEDI-5752, RO7121661, XmAb20717, and AK-104.

11. The combination of claim 10, wherein the immune checkpoint modulator is Pembrolizumab.

12. The combination of claim 8, wherein the immune checkpoint modulator is an antibody that binds to PD-L1, and the antibody that binds to PD-L1 is selected from the group consisting of Atezolizumab (MPDL3280A), Avelumab (MSB0010718C), Durvalumab (MEDI-4736), Envafolimab (KN035), AUNP12, CA-170, BMS-986189, BMS-936559, Cosibelimab (CK-301), LY3300054, CX-072, CBT-502, MSB-2311, BGB-A333, SHR-1316, CS1001 (WBP3155), HLX-20, KL-A167 (HBM 9167), STI-A1014, STI-A1015 (IMC-001), BCD-135, FAZ-053, CBT-502 (TQB2450), MDX1105-01, FS-118, M7824, CDX-527, LY3415244, and INBRX-105.

13. The combination of claim 12, wherein the immune checkpoint modulator is Atezolizumab.

14. (canceled)

15. (canceled)

16. (canceled)

17. (canceled)

18. (canceled)

19. (canceled)

20. The combination of claim 8, wherein the immune checkpoint modulator comprises an immune checkpoint activator.

21. (canceled)

22. (canceled)

23. The combination of claim 2, wherein the half-life extended immune cell engaging protein comprises a first domain (A), a second domain (B), and a third domain (C), wherein:
(i) the first domain (A) is the T cell engaging domain and specifically binds to human CD3,
(ii) the second domain (B) specifically binds to human serum albumin (HSA), and
(iii) the third domain (C) specifically binds to a target antigen;
wherein the domains are linked in one of the following orders: H₂N-(C)-(B)-(A)-COOH, H₂N-(A)-(B)-(C)-COOH, H₂N-(B)-(A)-(C)-COOH, H₂N-(C)-(A)-(B)-COOH, H₂N-(A)-(C)-(B)-COOH, or H₂N-(B)-(C)-(A)-COOH, or by linkers L1 and L2 in one of the following orders: H₂N-(C)-L1-(B)-L2(A)-COOH, H₂N-(A)-L1-(B)-L2-(C)-COOH, H₂N-(B)-L1-(A)-L2-(C)-COOH, H₂N-(C)-L1-(A)-L2-(B)-COOH, H₂N-(A)-L1-(C)-L2(B)-COOH, or H₂N-(B)-L1-(C)-L2-(A)-COOH.

24. The combination of claim 23, wherein the target antigen is a tumor antigen.

25-76. (canceled)

77. A composition comprising an immunomodulator and a half-life extended immune cell engaging protein.

78-91. (canceled)

92. A method of increasing the sensitivity of a subject to a therapy, comprising administering an immune checkpoint inhibitor, the method comprising administering to the subject a half-life extended immune cell engaging protein, comprising:
(i) a first domain (A) which specifically binds to human CD3,
(ii) a second domain (B) which specifically binds to human serum albumin (HSA), and
(iii) a third domain (C) which specifically binds to a target antigen.

93. The method of claim 92, wherein the administering the half-life extended immune cell engaging protein increases the concentration of an immune checkpoint protein targeted by the immune checkpoint inhibitor in the subject.

94. The method of claim 93, wherein the immune checkpoint protein is PD-1.

95. The method of claim 92, wherein the immune checkpoint inhibitor comprises an antibody selected from the group consisting of Pembrolizumab, Pidilizumab (CT-011), Spartalizumab (PDR001), Nivolumab (BMS-936558, MDX-1106), MEDI0680 (AMP-514), Cemiplimab (REGN2810), Dostarlimab (TSR-042), Sasanlimab (PF-06801591), Tislelizumab (BGB-A317), BGB-108, Tislelizumab (BGB-A317), Camrelizumab (INCSHR1210, SHR-1210), AMP-224, Zimberelimab (AB122, GLS-010, WBP-3055), AK-103 (HX-008), AK-105, CS1003, HLX10, Retifanlimab (MGA-012), BI-754091, Balstilimab (AGEN2034), toripalimab (JS-001), cetrelimab (JNJ-63723283), genolimzumab (CBT-501, anti-PD-1 antibody), LZM009, Prolgolimab (BCD-100), Sym021, ABBV-181, BAT-1306, JTX-4014, sintilimab (IBI-308), Tebotelimab (MGD013), MGD-019, KN-046, MEDI-5752, RO7121661, XmAb20717, and AK-104.

96. (canceled)

97. A method of improving the efficacy of a therapy in a subject in need thereof, comprising administering to the subject an immunomodulator, wherein the method further comprises administering to the subject a half-life extended immune cell engaging protein.

98. The method of claim 97, wherein the immunomodulator comprises an immunostimulatory antibody agonist of a co-stimulatory receptor.

99. The method of claim 97, wherein the immunomodulator comprises an immune checkpoint modulator.

100. The method of claim 99, wherein the immune checkpoint modulator is an antagonist of at least one of: programmed cell death 1 (PDCD1, PD1, PD-1), CD274 (CD274, PDL1, PD-L1), PD-L2, cytotoxic T-lymphocyte associated protein 4 (CTLA4, CD152), CD276 (B7H3); V-set domain containing T cell activation inhibitor 1 (VTCN1, B7H4), CD272 (B and T lymphocyte associated (BTLA)), killer cell immunoglobulin like receptor, three Ig domains and long cytoplasmic tail 1 (KIR, CD158E1), lymphocyte activating 3 (LAG3, CD223), hepatitis A virus cellular receptor 2 (HAVCR2, TEID3, TIM3), V-set immunoregulatory receptor (VSIR, B7H5, VISTA), T cell immunoreceptor with Ig and ITIM domains (TIGIT), programmed cell death 1 ligand 2 (PDCDILG2, PD-L2, CD273), immunoglobulin superfamily member 11 (IGSF11, VSIG3), TNFRSF14 (HVEM, CD270), TNFSF14 (HVEML), PVR related immunoglobulin domain containing (PVRIG, CD112R), galectin 9 (LGALS9), killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); a killer cell immunoglobulin like receptor, three Ig domains and long cytoplasmic tail 1 (KIR3DL1), killer cell lectin like receptor C1 (KLRC1, NKG2A, CD159A), killer cell lectin like receptor D1 (KLRD1, CD94), killer cell lectin like receptor G1 (KLRG1, CLEC15A, MAFA, 2F1), sialic acid binding Ig like lectin 7 (SIGLEC7), SIGLEC, sialic acid binding Ig like lectin 9 (SIGLEC9), CEACAM (e.g., CEACAM-1, CEACAM-3 and/or CEACAM-5), VISTA, LAIR1, CD160, 2B4, CD80, CD86, B7-H1, B7-H3 (CD276), B7-H4 (VTCN1), HVEM (TNFRSF14 or CD270), KIR, A2AR, A2BR, MHC class I, MHC class II, GAL9, adenosine, TGFR (e.g., TGFR beta), CD94/NKG2A, IDO, TDO, CD39, CD73, GARP, CD47, PVRIG, CSF1R, and NOX, or any combination thereof.

101. The method of claim 99, wherein the immune checkpoint modulator is an antagonist of PD-1, and the antagonist of PD-1 is selected from the group consisting of Pembrolizumab, Pidilizumab (CT-011), Spartalizumab (PDR001), Nivolumab (BMS-936558, MDX-1106), MEDI0680 (AMP-514), Cemiplimab (REGN2810), Dostarlimab (TSR-042), Sasanlimab (PF-06801591), Tislelizumab (BGB-A317), BGB-108, Tislelizumab (BGB-A317), Camrelizumab (INCSHR1210, SHR-1210), AMP-224, Zimberelimab (AB122, GLS-010, WBP-3055), AK-103 (HX-008), AK-105, CS1003, HLX10, Retifanlimab (MGA-012), BI-754091, Balstilimab (AGEN2034), toripalimab (JS-001), cetrelimab (JNJ-63723283), genolimzumab (CBT-501), LZM009, Prolgolimab (BCD-100), Sym021, ABBV-181, BAT-1306, JTX-4014, sintilimab (IBI-308), Tebotelimab (MGD013), MGD-019, KN-046, MEDI-5752, RO7121661, XmAb20717, and AK-104.

102. The method of claim 101, wherein the immune checkpoint modulator is Pembrolizumab.

103. The method of claim 99, wherein the immune checkpoint modulator is an antibody that binds to PD-L1, and the antibody that binds to PD-L1 is selected from the group consisting of Atezolizumab (MPDL3280A), Avelumab (MSB0010718C), Durvalumab (MEDI-4736), Envafolimab (KN035), AUNP12, CA-170, BMS-986189, BMS-936559, Cosibelimab (CK-301), LY3300054, CX-072, CBT-502, MSB-2311, BGB-A333, SHR-1316, CS1001 (WBP3155), HLX-20, KL-A167 (HBM 9167), STI-A1014, STI-A1015 (IMC-001), BCD-135, FAZ-053, CBT-502 (TQB2450), MDX1105-01, FS-118, M7824, CDX-527, LY3415244, and INBRX-105.

104. The method of claim 103, wherein the immune checkpoint modulator is Atezolizumab.

105-124. (canceled)
